(19) United States
(12) Patent Application Publication
(10) Pub. No.: US 2023/0357851 A1
(43) Pub. Date: Nov. 9, 2023

(54) FRATAXIN-SENSITIVE MARKERS FOR MONITORING FRATAXIN-REPLACEMENT THERAPY

(71) Applicant: Larimar Therapeutics, Inc., Bala Cynwyd, PA (US)

(72) Inventors: Joan David BETTOUN, Elkins Park, PA (US); Matthew Garrett BAILE, Wynnewood, PA (US); Ruihua CHEN, Kendall Park, NJ (US)

(21) Appl. No.: 18/296,966

(22) Filed: Apr. 6, 2023

Related U.S. Application Data
(60) Provisional application No. 63/393,968, filed on Jul. 31, 2022.
(60) Provisional application No. 63/328,238, filed on Apr. 6, 2022.

(51) Int. Cl. C12Q 1/6883

(52) U.S. Cl. CPC C12Q 1/6883; C12Q 2600/106; C12Q 2600/158

(57) ABSTRACT

The present disclosure is based, at least in part, on providing a set of markers, also referred to herein as FXN-sensitive genomic markers (or FSGMs), the respective expression levels of which are positively or negatively correlated to frataxin (FXN) levels in a cell. Therefore, these FSGMs can be used to determine, evaluate, and/or monitor the effectiveness of FXN replacement therapy in a subject.

### RELATED APPLICATIONS

This application claims priority to U.S. Provisional Patent Application No. 63/328,238, filed on Apr. 6, 2022 and U.S. Provisional Patent Application No. 63/393,968, filed on Jul. 31, 2022. The entire contents of each of the foregoing applications are hereby incorporated herein by reference.

### SEQUENCE LISTING

The instant application contains a Sequence Listing which has been submitted electronically in ASCII format and is hereby incorporated by reference in its entirety. Said ASCII copy, created on Apr. 6, 2023, is named 130197-01503_SL.xml and is 12,288 bytes in size.

### BACKGROUND

Mitochondrial diseases are a group of disorders caused by dysfunctional mitochondria, the cellular organelles that store potential energy in the form of adenosine triphosphate (ATP) molecules and are found in every cell of the human body except mature red blood cells.

Friedreich's Ataxia (FRDA) is the most common inherited ataxia in humans and results from a deficiency of the mitochondrial protein frataxin (FXN), and specifically human frataxin (hFXN). FRDA is a rare disease with an estimated incidence of 1:29,000, a carrier frequency of ˜1:85, and about 4,000-5,000 reported cases in the United States. FRDA is a progressive multisystem disease, typically beginning in mid-childhood. Subjects suffer from multiple symptoms, including progressive neurologic and cardiac dysfunction. Other clinical findings can include scoliosis, fatigue, diabetes, visual impairment, and hearing loss. Inheritance is autosomal recessive and is predominantly caused by an inherited GAA triplet expansion in the first intron of both alleles of the hFXN gene. This triplet expansion causes transcriptional repression of the FRDA gene, which results in the production of very small amounts of hFXN in subjects. hFXN heterozygotes typically have hFXN levels at ˜50% of normal but are phenotypically normal. hFXN levels of ˜45-70 pg/μl and ˜5-25 pg/μl in whole blood of heterozygotes and subjects afflicted with FRDA respectively have been shown to be stable over time (Plasterer et al., 2013).

Currently, there is no FDA-approved treatment for FRDA. Antioxidants and iron chelation have not been overly effective, and, despite treatment, subjects typically experience progressive loss of motor control and die, cardiomyopathy being the primary cause of death.

Protein replacement therapy is a well-established approach to metabolic diseases, such as diabetes, lysosomal storage disorders and hemophilia. Work in subject-derived cellular and animal models has demonstrated that replacement of functional FXN can correct or improve the FRDA disease phenotype. However, there is a need in the art for a reliable and efficient assay to measure clinical response and effectiveness of FXN replacement.

### SUMMARY

In one aspect, the present disclosure is based, at least in part, on providing a set of markers, also referred to herein as FXN-sensitive genomic markers (or FSGMs), whose respective levels are positively or negatively correlated to frataxin (FXN) levels in a cell.

In some embodiments, the FSGMs of the present disclosure are contrary regulated by FXN gene ablation or FXN deficiency in a subject followed by FXN protein replacement. Thus, said FSGMs of the present disclosure are both associated with FXN deficiency in a subject and conversely associated with FXN replacement. The FSGMs disclosed herein were found to be sensitive to FXN levels and are considered markers of FXN deficiency, and of FXN replacement therapy.

Therefore, any one or more of the FSGMs provided herein can serve as surrogate biomarker for FXN levels in a subject. For example, the FSGMs provided herein can be used to evaluate or to monitor progression of an FXN deficiency in a subject, as described herein. Further, the FSGMs provided herein can be used to evaluate, and/or monitor an FXN replacement therapy, e.g., determine, evaluate and/or monitor the efficacy of FXN replacement therapy, in a subject, as described herein.

In some embodiments, an FXN replacement therapy, e.g., efficacy of an FXN replacement therapy, in a subject can be determined, evaluated, and/or monitored based on the analysis of one or more FSGM expression profiles in samples obtained from a subject before, during and/or after administration or initiation of FXN replacement therapy to the subject. Based on the results of the FSGM expression profile analysis, adjustments can be made to the FXN replacement therapy in a subject, such as to, e.g., initiate an FXN replacement therapy, increase a dose and/or administration frequency of an FXN replacement therapy, decrease a dose and/or administration frequency of an FXN replacement therapy, or cease FXN replacement therapy in the subject.

In some aspects, the present disclosure provides a method for evaluating efficacy of a frataxin (FXN) replacement therapy, the method comprising: (a) determining a baseline FXN(−) expression profile for one or more FXN-sensitive genomic markers (FSGMs) in a sample obtained from an FXN deficient subject prior to administration of the FXN replacement therapy; (b) determining an FXN replacement expression profile for the one or more FXN-sensitive genomic markers (FSGMs) in a sample obtained from the FXN deficient subject following administration of the FXN replacement therapy; (c) comparing the FXN replacement expression profile determined in step (b) with the baseline FXN(−) expression profile determined in step (a); and (d) determining efficacy of the FXN replacement therapy based on the comparison in step (c); wherein the one or more FSGMs are selected from the markers defined in Table 1.

In some aspects, the present disclosure provides a method for evaluating efficacy of a frataxin (FXN) replacement therapy, the method comprising: (a) determining an FXN replacement expression profile for one or more FXN-sensitive genomic markers (FSGMs) in a sample obtained from an FXN deficient subject following administration of an FXN replacement therapy; (b) comparing the subject FXN replacement expression profile determined in step (a) with a reference FXN expression profile for the one or more FSGMs; and (c) determining efficacy of the FXN replacement therapy based on the comparison in step (b); wherein the one or more FSGMs are selected from the markers defined in Table 1.

In some embodiments, the reference FXN expression profile is a baseline FXN(−) expression profile for the one or more FSGMs. In other embodiments, the reference FXN expression profile is a normal FXN expression profile for the one or more FSGMs.

In some embodiments, the baseline FXN(−) expression profile for the one or more FSGMs is determined in a sample obtained from an FXN deficient subject prior to administration of an FXN replacement therapy.

In some embodiments, the method further comprises determining a baseline FXN(−) expression profile for the one or more FXN-sensitive genomic markers (FSGMs) in a sample obtained from the FXN deficient subject prior to administration of the FXN replacement therapy.

In some embodiments, the one or more FSGMs comprise one or more of Timp1, LRRK2, BASP1, CD44, Xpo6, Lgals3, SOD2, PSMB9, FTL and TXNIP. In some embodiments, the one or more FSGMs comprise any 2, 3, 4, 5, 6, 7, 8, 9 or all 10 of Timp1, LRRK2, BASP1, CD44, Xpo6, Lgals3, SOD2, PSMB9, FTL and TXNIP. In some embodiments, the one or more FSGMs comprise one or more of Timp1, LRRK2, BASP1, CD44, Xpo6, Lgals3, PSMB9, FTL and TXNIP.

In some embodiments, the one or more FSGMs comprise one or more of Timp1, LRRK2, BASP1, CD44, Xpo6, Lgals3, SOD2 and PSMB9. In some embodiments, the one or more FSGMs comprise any 2, 3, 4, 5, 6, 7 or all 8 of Timp1, LRRK2, BASP1, CD44, Xpo6, Lgals3, SOD2 and PSMB9. In some embodiments, the one or more FSGMs comprise one or more of Timp1, LRRK2, BASP1, CD44, Xpo6, Lgals3, and PSMB9.

In some embodiments, the one or more FSGMs further comprise one or more FSGMs defined in Table 2 or Table 3. In some embodiments, the one or more FSGMs defined in Table 2 or Table 3 comprise 1, 2, 3 or all 4 of CTSS, BTG2, EGR1 and PTGS2. In some embodiments, the one or more FSGMs comprise Timp1, LRRK2, BASP1, CD44, Xpo6, Lgals3, SOD2, PSMB9, FTL, TXNIP, CTSS, BTG2, EGR1 and PTGS2. In some embodiments, the one or more FSGMs comprise Xpo6, SOD2, CTSS, EGR1, BTG2, PTSG2 and LRKK2. In some embodiments, the one or more FSGMs comprise Timp1, LRRK2, BASP1, CD44, Xpo6, Lgals3, PSMB9, CTSS, BTG2, EGR1 and PTGS2.

In some embodiments, the one or more FSGMs further comprise one or more FSGMs defined in Table 2 or Table 3. In some embodiments, the one or more FSGMs defined in Table 2 or Table 3 comprise 1, 2, 3 or all 4 of CTSS, BTG2, EGR1 and PTGS2. In some embodiments, the one or more FSGMs comprise Timp1, LRRK2, BASP1, CD44, Xpo6, Lgals3, SOD2, PSMB9, CTSS, BTG2, EGR1 and PTGS2. In some embodiments, the one or more FSGMs comprise Xpo6, SOD2, CTSS, EGR1, BTG2, PTSG2 and LRKK2. In some embodiments, the one or more FSGMs comprise Timp1, LRRK2, BASP1, CD44, Xpo6, Lgals3, PSMB9, CTSS, BTG2, EGR1 and PTGS2. In some embodiments, the one or more FSGMs comprise Xpo6, SOD2, FTL, TXNIP, CTSS, EGR1, BTG2, PTSG2 and LRKK2. In some embodiments, the one or more FSGMs comprise Timp1, LRRK2, BASP1, CD44, Xpo6, Lgals3, PSMB9, FTL, TXNIP, CTSS, BTG2, EGR1 and PTGS2.

In some embodiments, at least one or more FSGMs are upregulated following treatment with FXN replacement therapy. In some embodiments, the one or more FSGMs upregulated following treatment with FXN replacement therapy comprise one or more of LY96, LRRK2, Adgre1, Timp1, Xpo6, CD44, BASP1, PSMB8, C3ar1, TFRC, SOD2, PLCL2, S100a4, Mapre1, PSMB9, FTL, TXNIP, CFH and Tmem70. In some embodiments, the one or more FSGMs upregulated following treatment with FXN replacement therapy comprise one or more of Timp1, LRRK2, BASP1, CD44, Xpo6, SOD2, PSMB9, FTL, TXNIP, CTSS, BTG2, EGR1 and PTGS2.

In some embodiments, at least one or more FSGMs are upregulated following treatment with FXN replacement therapy. In some embodiments, the one or more FSGMs upregulated following treatment with FXN replacement therapy comprise one or more of LY96, LRRK2, Adgre1, Timp1, Xpo6, Cd44, BASP1, PSMB8, C3ar1, TFRC, SOD2, PLCL2, S100a4, Mapre1, PSMB9, CFH and Tmem70. In some embodiments, the one or more FSGMs upregulated following treatment with FXN replacement therapy comprise one or more of Timp1, LRRK2, BASP1, CD44, Xpo6, SOD2, PSMB9, CTSS, BTG2, EGR1 and PTGS2.

In some embodiments, the one or more FSGMs upregulated following treatment with FXN replacement therapy comprise LY96. In some embodiments, the one or more FSGMs upregulated following treatment with FXN replacement therapy comprise LRRK2. In some embodiments, the one or more FSGMs upregulated following treatment with FXN replacement therapy comprise Adgre1. In some embodiments, the one or more FSGMs upregulated following treatment with FXN replacement therapy comprise Timp1. In some embodiments, the one or more FSGMs upregulated following treatment with FXN replacement therapy comprise Xpo6. In some embodiments, the one or more FSGMs upregulated following treatment with FXN replacement therapy comprise CD44.

In some embodiments, the one or more FSGMs upregulated following treatment with FXN replacement therapy comprise BASP1. In some embodiments, the one or more FSGMs upregulated following treatment with FXN replacement therapy comprise PSMB8. In some embodiments, the one or more FSGMs upregulated following treatment with FXN replacement therapy comprise C3ar1. In some embodiments, the one or more FSGMs upregulated following treatment with FXN replacement therapy comprise TFRC. In some embodiments, the one or more FSGMs upregulated following treatment with FXN replacement therapy comprise SOD2. In some embodiments, the one or more FSGMs upregulated following treatment with FXN replacement therapy comprise PLCL2. In some embodiments, the one or more FSGMs upregulated following treatment with FXN replacement therapy comprise S100a4. In some embodiments, the one or more FSGMs upregulated following treatment with FXN replacement therapy comprise Mapre1. In some embodiments, the one or more FSGMs upregulated following treatment with FXN replacement therapy comprise PSMB9. In some embodiments, the one or more FSGMs upregulated following treatment with FXN replacement therapy comprise FTL. In some embodiments, the one or more FSGMs upregulated following treatment with FXN replacement therapy comprise TXNIP. In some embodiments, the one or more FSGMs upregulated following treatment with FXN replacement therapy comprise CFH. In some embodiments, the one or more FSGMs upregulated following treatment with FXN replacement therapy comprise Tmem70.

In some embodiments, at least one or more FSGMs are downregulated following treatment with FXN replacement therapy. In some embodiments, the one or more FSGMs downregulated following treatment with FXN replacement therapy comprise one or more of Lgals3, Ube2v2, Sec61g, Zdhhc13, Yipf5, Tpp2, Ybx2 and Ptms. In some embodiments, the one or more FSGMs downregulated following treatment with FXN replacement therapy comprise Lgals3.

In some embodiments, the one or more FSGMs downregulated following treatment with FXN replacement therapy comprise Lgals3. In some embodiments, the one or more FSGMs downregulated following treatment with FXN replacement therapy comprise Ube2v2. In some embodiments, the one or more FSGMs downregulated following treatment with FXN replacement therapy comprise Sec61g. In some embodiments, the one or more FSGMs downregulated following treatment with FXN replacement therapy comprise Zdhhc13. In some embodiments, the one or more FSGMs downregulated following treatment with FXN replacement therapy comprise Yipf5.

In some embodiments, the one or more FSGMs downregulated following treatment with FXN replacement therapy comprise Tpp2. In some embodiments, the one or more FSGMs downregulated following treatment with FXN replacement therapy comprise Ybx2. In some embodiments, the one or more FSGMs downregulated following treatment with FXN replacement therapy comprise Ptms.

In some embodiments, determining an FXN expression profile for one or more FSGMs comprises detecting the level of expression of the one or more FSGMs. In some embodiments, comparing the subject FXN replacement expression profile with the baseline FXN(−) expression profile comprises comparing the level of expression of the one or more FSGMs in the FXN replacement expression profile with the level of expression of the corresponding one or more FSGMs in the baseline FXN(−) expression profile.

In some embodiments, when the expression level of one or more of Timp1, LRRK2, BASP1, CD44, Xpo6, SOD2, PSMB9, FTL, TXNIP, CTSS, BTG2, EGR1 and PTGS2 is increased in the FXN replacement expression profile as compared to the baseline FXN(−) expression profile, the FXN replacement therapy is determined to be effective. In some embodiments, when the expression level of Lgals3 is decreased in the FXN replacement expression profile as compared to the baseline FXN(−) expression profile, the FXN replacement therapy is determined to be effective.

In some embodiments, determining an FXN expression profile for one or more FSGMs comprises determining an FXN feature vector of values indicative of expression of the one or more FSGMs. In some embodiments, determining efficacy of the FXN replacement therapy comprises determining a first FXN feature vector for the subject FXN replacement expression profile and a second FXN feature vector for the baseline FXN (−) expression profile and determining a distance between the first and second feature vectors. In some embodiments, determining the distance between the feature vectors comprises determining a scalar product of the first and second feature vectors.

In some embodiments, determining an FXN expression profile for one or more FSGMs further comprises determining a third feature vector for a normal FXN expression profile for the FSGMs for a healthy subject. In some embodiments, determining an FXN expression profile for one or more FSGMs further comprises determining a distance between the second and third feature vectors. In some embodiments, determining an FXN expression profile for one or more FSGMs further comprises determining a distance between the first and third feature vectors, and normalizing the distance between the first and third feature vectors to the distance between the second and third feature vectors. In some embodiments, determining an FXN expression profile for one or more FSGMs further comprises using the normalized distance to determine effectiveness of the FXN replacement therapy.

In some embodiments, the FXN expression profile is determined by one or more of sequencing, hybridization and amplification of RNA in the sample. In some embodiments, the FXN expression profile is determined by HPLC/UV-Vis spectroscopy, enzymatic analysis, mass spectrometry, NMR, immunoassay, ELISA, or any combination thereof. In some embodiments, the FXN expression profile is determined by determining an amount of expressed protein by one or more of HPLC/UV-Vis spectroscopy, enzymatic analysis, mass spectrometry, NMR, immunoassay, ELISA, or any combination thereof.

In some embodiments, the methods of the disclosure further comprise recommending to a healthcare provider to modify the treatment with the FXN replacement therapy based on the determination of efficacy for the FXN replacement therapy. In some embodiments, the subject has Freidrich's Ataxia (FRDA).

In some embodiments, the methods of the disclosure comprise obtaining a sample from the FXN deficient subject. In some embodiments, the sample is selected from the group consisting of a buccal sample, a skin sample, a hair follicle and a muscle biopsy sample. In some embodiments, the sample is a buccal sample. In some embodiments, the sample is a skin sample.

In some embodiments, the sample from the FXN deficient subject is obtained at least 15 days following the last administration of the FXN replacement therapy. In some embodiments, the sample from the FXN deficient subject is obtained 15 to 45 days following the last administration of the FXN replacement therapy.

In some aspects, the present disclosure provides a method of monitoring treatment of a subject with a frataxin (FXN) replacement therapy, the method comprising: (a) determining a first FXN replacement expression profile for one or more FXN-sensitive genomic markers (FSGMs) in a first sample obtained from an FXN deficient subject at a first time point following administration of an FXN replacement therapy to the subject, wherein the one or more FSGMs comprises one or more markers defined in Table 1; (b) determining a second FXN replacement expression profile for the one or more FXN-sensitive genomic markers (FSGMs) in a second sample obtained from the subject at a second time point that is later than the first time point; (c) comparing the second FXN replacement expression profile with the first FXN replacement profile; thereby monitoring treatment of the subject with the FXN replacement therapy.

In some embodiments, the method further comprises making a determination to maintain, increase or decrease the dose or administration frequency of the FXN replacement therapy based on the comparison in step (c). In some embodiments, at least one dose of the FXN replacement therapy is administered to the subject between obtaining the first time point and second time point. In some embodiments, the FXN replacement therapy is not administered to the subject between obtaining the first time point and second time point.

In some aspects, the present disclosure provides a method for treating an FXN deficiency, the method comprising: (a) determining an FXN expression profile in a sample obtained from an FXN deficient subject for one or more FXN-sensitive genomic markers (FSGMs), (b) comparing the FXN expression profile of the sample with at least one other expression profile selected from the group consisting of normal FXN expression profile for the one or more FSGMs, baseline FXN(−) expression profile for the one or more FSGMs, and FXN replacement expression profile for the one or more FSGMs, (c) classifying the FXN expression profile determined in step (a) as corresponding to a normal FXN expression profile, baseline FXN(−) expression profile or an FXN replacement expression profile, and (d) initiating or modulating an FXN replacement therapy based on the classification of the FXN expression profile of the sample, wherein the one or more FSGMs comprises one or more markers defined in Table 1.

In some embodiments, modulating an FXN replacement therapy comprises increasing the dosage, decreasing the dosage, increasing the administration frequency, or decreasing the administration frequence, of the FXN replacement therapy. In some embodiments, the FXN deficient subject is Friedrich's Ataxia (FRDA).

In some aspects, the present disclosure provides a method of treating an FXN deficiency in a subject, comprising: (a) determining an FXN expression profile for one or more FSGMs in a sample from an FXN deficient subject; and (b) recommending to a healthcare provider to administer an FXN replacement therapy to the subject based on the subject FXN expression profile determined in step (a).

In some aspects, the present disclosure provides a method of treating an FXN deficiency in a subject, comprising: (a) obtaining an FXN expression profile for one or more FSGMs in a sample obtained from an FXN deficient subject; and (b) administering an FXN replacement therapy to the subject based on the subject FXN expression profile.

In some embodiments, the methods of the disclosure further comprise obtaining the sample from the FXN deficient subject for use in determining the FXN expression profile for the one or more FSGMs.

In some aspects, the present disclosure provides a method of detecting one or more frataxin-sensitive genomic markers (FSGMs) in a sample from a frataxin (FXN) deficient subject, comprising: contacting the sample, or a portion thereof, with one or more reagents specific for detecting the level of each of one or more FSGMs, wherein the one or more FSGMs comprise one or more FSGMs selected from Table 1, thereby detecting the FSGMs in the sample.

In some embodiments, the subject is being treated or is scheduled to be treated with an FXN replacement therapy.

In some embodiments, the method further comprises obtaining the sample from the FXN deficient subject.

In some embodiments, the one or more FSGMs comprise one or more of Timp1, LRRK2, BASP1, CD44, Xpo6, Lgals3, SOD2, PSMB9, FTL and TXNIP. In some embodiments, the one or more FSGMs comprise Timp1, LRRK2, BASP1, CD44, Xpo6, Lgals3, PSMB9, FTL and TXNIP.

In some embodiments, the one or more FSGMs comprise one or more of Timp1, LRRK2, BASP1, CD44, Xpo6, Lgals3, SOD2 and PSMB9. In some embodiments, the one or more FSGMs comprise Timp1, LRRK2, BASP1, CD44, Xpo6, Lgals3, and PSMB9.

In some embodiments, the one or more FSGMs further comprise one of more FSGMs defined in Table 2 or Table 3. In some embodiments, the one or more FSGMs defined in Table 2 or Table 3 comprise one or more of CTSS, BTG2, EGR1 and PTGS2. In some embodiments, the one or more FSGMs comprise Xpo6, SOD2, CTSS, EGR1, BTG2, PTSG2 and LRKK2. In some embodiments, the one or more FSGMs comprise Xpo6, SOD2, FTL, TXNIP, CTSS, EGR1, BTG2, PTSG2 and LRKK2.

In some embodiments, the FXN expression profile is determined by one or more of sequencing, hybridization and amplification of RNA in the sample. In some embodiments, the FXN expression profile is determined by HPLC/UV-Vis spectroscopy, enzymatic analysis, mass spectrometry, NMR, immunoassay, ELISA, or any combination thereof. In some embodiments, the FXN expression profile is determined by measuring an amount of expressed protein by HPLC/UV-Vis spectroscopy, enzymatic analysis, mass spectrometry, NMR, immunoassay, ELISA, or any combination thereof.

In some embodiments, the subject has Freidrich's Ataxia (FRDA).

In some embodiments, methods of the disclosure further comprise obtaining a sample from the FXN deficient subject. In some embodiments, the sample is selected from the group consisting of a buccal sample, a skin sample, a hair follicle and a muscle biopsy sample. In some embodiments, the sample is a buccal sample. In some embodiments, the sample is a skin sample.

In some embodiments, the sample from the FXN deficient subject is obtained at least 15 days following the last administration of the FXN replacement therapy. In some embodiments, the sample from the FXN deficient subject is obtained 15 to 45 days following the last administration of the FXN replacement therapy.

In some embodiments, the FXN replacement therapy comprises administration of an FXN fusion protein. In some embodiments, the FXN fusion protein comprises or consists of the amino acid sequence set forth in SEQ ID NO: 12.

In some aspects, the present disclosure also provides a kit for detecting one or more frataxin-sensitive genomic markers (FSGMs) in a sample obtained from a frataxin (FXN) deficient subject, comprising at least one reagent specific for detecting the level of each of the one or more FSGMs in the sample, wherein the one or more FSGMs comprises one or more FSGMs defined in Table 1, and a set of instructions for detecting the level of the one or more FSGMs in the sample from the subject.

In some embodiments, the reagent is an antibody that binds to the frataxin-sensitive genomic marker (FSGM) or an oligonucleotide that is complementary to the corresponding mRNA of the FSGM.

In some embodiments, the one or more FSGMs comprise one or more of Timp1, LRRK2, BASP1, CD44, Xpo6, Lgals3, SOD2, PSMB9, FTL and TXNIP.

In some embodiments, the one or more FSGMs comprise one or more of Timp1, LRRK2, BASP1, CD44, Xpo6, Lgals3, SOD2 and PSMB9.

In some embodiments, the kit of the disclosure further comprises at least one reagent specific for detecting the level of one of more FSGMs defined in Table 2 or Table 3. In some embodiments, the one or more FSGMs defined in Table 2 or Table 3 comprise one or more of CTSS, BTG2, EGR1 and PTGS2. In some embodiments, the one or more FSGMs comprise Xpo6, SOD2, CTSS, EGR1, BTG2, PTSG2 and LRKK2. In some embodiments, the one or more FSGMs comprise Xpo6, SOD2, FTL, TXNIP, CTSS, EGR1, BTG2, PTSG2 and LRKK2.

In some aspects, the present disclosure provides a panel of reagents for use in a method of monitoring or evaluating the efficacy of frataxin (FXN) replacement therapy, the panel comprising at least two detection reagents, wherein each detection reagent is specific for the detection of at least one frataxin-sensitive genomic marker (FSGM) of a set of FSGMs, wherein the set of FSGMs comprises two or more markers defined in Table 1.

In some embodiments, the set of FSGMs comprise two or more of Timp1, LRRK2, BASP1, CD44, Xpo6, Lgals3, SOD2, PSMB9, FTL and TXNIP. In some embodiments, the set of FSGMs comprise two or more of Timp1, LRRK2, BASP1, CD44, Xpo6, Lgals3, SOD2 and PSMB9.

In some embodiments, the panel of the disclosure further comprises at least one detection reagents specific for the detection of at least one FSGM of the FSGMs defined in Table 2 or Table 3. In some embodiments, the at least one of the FSGMs defined in Table 2 or Table 3 comprise one or more of CTSS, BTG2, EGR1 and PTGS2. In some embodiments, the at least one of the FSGMs comprise one or more of Xpo6, SOD2, CTSS, EGR1, BTG2, PTSG2 and LRKK2. In some embodiments, the at least one of the FSGMs comprise Xpo6, SOD2, CTSS, EGR1, BTG2, PTSG2 and LRKK2. In some embodiments, the at least one of the FSGMs comprise one or more of Xpo6, SOD2, FTL, TXNIP, CTSS, EGR1, BTG2, PTSG2 and LRKK2. In some embodiments, the at least one of the FSGMs comprise Xpo6, SOD2, FTL, TXNIP, CTSS, EGR1, BTG2, PTSG2 and LRKK2.

In some aspects, the present disclosure provides a kit comprising the panel of any one of claims 76-80 and a set of instructions for obtaining information relating to frataxin (FXN) replacement therapy based on a level of the one or more frataxin-sensitive genomic markers (FSGMs).

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” (having the meaning of and/or) rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear.

FIG. 1 is a volcano plot resulting from principal component analysis of buccal samples obtained from healthy subjects vs. buccal samples obtained from FRDA subjects who were not treated with FXN replacement therapy. FIG. 1 shows expression levels of various gene markers vs. their corresponding adjusted p-values.

FIG. 2 is a graph showing the relative levels of selected gene markers in healthy subjects (“Normal”) and FRDA subjects treated with placebo (“FA placebo”).

FIG. 3 is a volcano plot resulting from principal component analysis of samples from subjects treated with 100 mg of the exemplary FXN fusion protein obtained pre-dose (day −2) and on day 22. FIG. 3 shows expression levels of various gene markers vs. their corresponding adjusted p-values.

FIG. 4 is a graph showing relative amounts of selected gene markers in buccal samples obtained from healthy subjects (“Normal”) and FRDA subjects on day 22 after treatment with placebo (“FA placebo”), 25 mg (“FA cohort 1”), 50 mg (“FA cohort 2”) and 100 mg (“FA cohort 3”) of the exemplary FXN fusion protein.

FIG. 5 is a graph showing the ratio of the relative amounts of selected gene markers in FRDA subjects treated with placebo (“placebo”), 25 mg (“cohort 1”), 50 mg (“cohort 2”) and 100 mg (“cohort 3”) of the exemplary FXN fusion protein on day 22 after dosing as compared to pre-dose.

FIG. 6 is a graph showing the relative amounts of selected FSGMs on day 2, day 15, day 22 and day 43 measured in buccal samples taken after dosing FRDA subjects with placebo or 100 mg of the exemplary FXN fusion protein (“cohort 3”).

FIG. 7 is a volcano plot resulting from principal component analysis of whole blood samples obtained from healthy subjects vs. FRDA subjects who were not treated with FXN replacement therapy.

FIG. 8 is a box plot showing relative amounts of FTL and TXNLP gene markers in buccal samples obtained from healthy subjects (“Normal”) and FRDA subjects on day 22 after treatment with placebo (“FA placebo”) and 100 mg (“FA cohort 3”) of the exemplary FXN fusion protein.

FIG. 9 is a box plot showing the ratio of the relative amounts of gene markers in FRDA subjects treated with placebo (“placebo”) and 100 mg (“cohort 3”) of the exemplary FXN fusion protein on day 22 after dosing as compared to pre-dose.

### DETAILED DESCRIPTION OF THE INVENTION

### A. Overview

In one aspect, the present disclosure is based, at least in part, on providing a set of markers, also referred to herein as FXN-sensitive genomic markers (or FSGMs), whose respective levels are positively or negatively correlated to frataxin (FXN) levels in a cell. In some embodiments, the FSGMs of the present disclosure are contrary regulated by FXN gene ablation or deficiency, followed by FXN protein replacement. Thus, said FSGMs of the present disclosure are both associated with FXN deficiency in a subject and conversely associated with FXN replacement. The FSGMs disclosed herein were found to be sensitive to FXN and are considered markers of FXN replacement. Therefore, these FSGMs can be used to evaluate and/or monitor progression of FXN deficiency in a subject, as described herein. These FSGMs can also be used to determine and/or monitor efficacy of FXN replacement therapy in a subject, as described herein. In one embodiment, the FSGMs comprise one or more markers defined in Table 1 and/or Table 5.

In one embodiment, the one or more FSGMs comprise one or more of Timp1, LRRK2, BASP1, CD44, Xpo6, Lgals3, SOD2, PSMB9, FTL and TXNIP. In another embodiment, the one or more FSGMs comprise two or more of Timp1, LRRK2, BASP1, CD44, Xpo6, Lgals3, SOD2, PSMB9, FTL and TXNIP. In another embodiment, the one or more FSGMs comprise three or more of Timp1, LRRK2, BASP1, CD44, Xpo6, Lgals3, SOD2, PSMB9, FTL and TXNIP. In another embodiment, the one or more FSGMs comprise four or more of Timp1, LRRK2, BASP1, CD44, Xpo6, Lgals3, SOD2, PSMB9, FTL and TXNIP. In another embodiment, the one or more FSGMs comprise five or more of Timp1, LRRK2, BASP1, CD44, Xpo6, Lgals3, SOD2, PSMB9, FTL and TXNIP. In another embodiment, the one or more FSGMs comprise six or more of Timp1, LRRK2, BASP1, CD44, Xpo6, Lgals3, SOD2, PSMB9, FTL and TxNIP. In another embodiment, the one or more FSGMs comprise seven or more of Timp1, LRRK2, BASP1, CD44, Xpo6, Lgals3, SOD2, PSMB9, FTL and TXNIP. In another embodiment, the one or more FSGMs comprise eight or more of Timp1, LRRK2, BASP1, CD44, Xpo6, Lgals3, SOD2, PSMB9, FTL and TXNIP. In another embodiment, the one or more FSGMs comprise Timp1, LRRK2, BASP1, CD44, Xpo6, Lgals3, SOD2, PSMB9, FTL and TXNIP.

In one embodiment, the one or more FSGMs comprise one or more of Timp1, LRRK2, BASP1, CD44, Xpo6, Lgals3, SOD2 and PSMB9. In another embodiment, the one or more FSGMs comprise two or more of Timp1, LRRK2, BASP1, CD44, Xpo6, Lgals3, SOD2 and PSMB9. In another embodiment, the one or more FSGMs comprise three or more of Timp1, LRRK2, BASP1, CD44, Xpo6, Lgals3, SOD2 and PSMB9. In another embodiment, the one or more FSGMs comprise four or more of Timp1, LRRK2, BASP1, CD44, Xpo6, Lgals3, SOD2 and PSMB9. In another embodiment, the one or more FSGMs comprise five or more of Timp1, LRRK2, BASP1, CD44, Xpo6, Lgals3, SOD2 and PSMB9. In another embodiment, the one or more FSGMs comprise six or more of Timp1, LRRK2, BASP1, CD44, Xpo6, Lgals3, SOD2 and PSMB9. In another embodiment, the one or more FSGMs comprise seven or more of Timp1, LRRK2, BASP1, CD44, Xpo6, Lgals3, SOD2 and PSMB9. In another embodiment, the one or more FSGMs comprise Timp1, LRRK2, BASP1, CD44, Xpo6, Lgals3, SOD2 and PSMB9.

In one embodiment, the one or more FSGMs comprise one or more of LRRK2, SOD2, CD44, and Lgals3. In another embodiment, the one or more FSGMs comprise two or more of LRRK2, SOD2, CD44, and Lgals3. In another embodiment, the one or more FSGMs comprise three or more of LRRK2, SOD2, CD44, and Lgals3. In another embodiment, the one or more FSGMs comprise LRRK2, SOD2, CD44, and Lgals3.

In one embodiment, the one or more FSGMs comprise one or more of LRRK2, SOD2, CD44, Lgals3, FTL and TXNIP. In another embodiment, the one or more FSGMs comprise two or more of LRRK2, SOD2, CD44, Lgals3, FTL and TXNIP. In another embodiment, the one or more FSGMs comprise three or more of LRRK2, SOD2, CD44, Lgals3, FTL and TXNIP. In another embodiment, the one or more FSGMs comprise four or more of LRRK2, SOD2, CD44, Lgals3, FTL and TXNIP. In another embodiment, the one or more FSGMs comprise five or more of LRRK2, SOD2, CD44, Lgals3, FTL and TXNIP. In another embodiment, the one or more FSGMs comprise LRRK2, SOD2, CD44, Lgals3, FTL and TXNIP.

In another embodiment, the one or more FSGMs further comprise one or more FSGMs defined in Table 2 and/or Table 3. In one embodiment, the one or more FSGMs defined in Table 2 and/or Table 3 comprise one or more of CTSS, BTG2, EGR1, and PTGS2. In another embodiment, the one or more FSGMs defined in Table 2, 3, 4 and/or Table 6 comprise two or more of BTG2, EGR1, and PTGS2. In another embodiment, the one or more FSGMs defined in Table 2 and/or Table 3 comprise three or more of CTSS, BTG2, EGR1, and PTGS2. In another embodiment, the one or more FSGMs defined in Table 2, 3, 4 and/or Table 6 comprise CTSS, BTG2, EGR1, and PTGS2.

In some embodiments, the efficacy of FXN replacement therapy in a subject can be determined, evaluated, and/or monitored based on the analysis of one or more FSGM expression profiles before and after administration or initiation of FXN replacement therapy in the subject. Based on the results of the FSGM expression profile analysis, adjustments can be made to the FXN replacement therapy in a subject to, e.g., initiate, increase, decrease or cease FXN replacement therapy in the subject.

### B. Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “amplification” refers to any known in vitro procedure for obtaining multiple copies (“amplicons”) of a target nucleic acid sequence or its complement or fragments thereof. In vitro amplification refers to production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating RNA molecules, and a replicase such as Q-β-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. Nos. 6,087,133 and 6,124,120 (MSDA)). Those skilled in the art will understand that the oligonucleotide primer sequences of the present invention may be readily used in any in vitro amplification method based on primer extension by a polymerase. (see generally Kwoh et al., 1990, Am. Biotechnol. Lab. 8:14-25 and (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 2000, Molecular Cloning—A Laboratory Manual, Third Edition, CSH Laboratories). As commonly known in the art, the oligos are designed to bind to a complementary sequence under selected conditions.

As used herein, the term “marker” or “biomarker” is a biological molecule, or a panel of biological molecules, whose expression level is correlated, e.g., either positively or negatively, with FXN levels. In some embodiments, a “marker” or a “biomarker” is an expressed gene, whose expression level may be measured by measuring levels of the corresponding mRNA or a protein.

As used herein, a marker or biomarker of the invention whose respective levels are positively or negatively correlated to frataxin (FXN) levels in a cell or a sample obtained from a subject is referred to as a “Frataxin-sensitive genomic marker” or “FSGM”.

In some embodiments, an FSGM is a marker or a biomarker, e.g., an expressed gene, that is differentially expressed in samples obtained from a healthy subject as compared to a sample obtained from an FXN-deficient subject, e.g., subjects with FRDA. In further embodiments, an FSGM is contrary regulated by FXN gene ablation or deficiency, followed by FXN replacement therapy. For example, an FSGM may be a gene, e.g., Timp1, that is expressed at a lower level in a sample obtained from an FXN-deficient subject as compared to a sample obtained from a healthy subject, and levels of which increase in a sample from an FXN-deficient subject following FXN replacement therapy. Alternatively, for example, an FSGM may be a gene, e.g., Lgals3, that is expressed at a higher level in a sample obtained an FXN-deficient subject as compared to a sample obtained from a healthy subject, and levels of which decrease in a sample from an FXN-deficient subject following FXN replacement therapy.

In some embodiments, an FSGM is a marker or a biomarker, e.g., an expressed gene, that is differentially expressed in a sample obtained from a healthy subject as compared to a sample obtained from an FXN-deficient subject, as determined by principal component analysis, that shows an expression fold change of greater than 2 between groups, with Benjamini-Hochbert (BH) adjusted p-value <0.05.

In some embodiments, an FSGM is a marker or a biomarker, e.g., an expressed gene, that is differentially expressed in a sample obtained from a healthy subject as compared to a sample obtained from an FXN-deficient subject, as determined by principal component analysis, that shows an expression fold change of less than 2, e.g., between 1 and 2, between groups, with Benjamini-Hochbert (BH) adjusted p-value <0.05.

In some embodiments, an FSGM is a marker or a biomarker, e.g., an expressed gene, that is differentially expressed in a sample obtained from a healthy subject as compared to a sample obtained from an FXN-deficient subject, as determined by principal component analysis, that shows an expression fold change of greater than 2 between groups, with Benjamini-Hochbert (BH) adjusted p-value >0.05.

In some embodiments, the FSGMs of the present disclosure are contrary regulated by FXN gene ablation or deficiency, followed by FXN protein replacement. Thus, in some embodiments, the FSGMs of the present disclosure are both associated with FXN deficiency in a subject and conversely associated with FXN replacement. An FSGM of the invention can be used to detect and/or monitor (e.g., serve as a surrogate for) FXN levels in a sample, e.g., a cell or tissue sample.

In preferred embodiments, an FSGM is selected from those listed in Table 1 and/or Table 5, human genes and proteins in Table 1 and/or Table 5, and human homologues of genes and proteins in Table 1 and/or Table 5. In some embodiments, the FSGMs are selected from those listed in Table 1 and/or Table 5 in combination with one or more additional FSGMs in Table 2 and/or Table 3, human genes and proteins in Table 1 and/or Table 5, and human homologues of genes and proteins in Table 1 and/or Table 5. Reference to FSGMs in Table 1 and/or Table 5, alone or in combination with one or more additional FSGMs in Table 2 and/or Table 3, as used herein, is understood to include reference to any mutants, variants, derivatives, or orthologs thereof.

The term “control sample” or “control,” as used herein, refers to any clinically relevant comparative sample, or comparative expression profile, including, for example, a sample from an FXN healthy subject (i.e., a subject with a normal FXN level), a normal FXN expression profile, a sample from an FXN deficient subject (i.e., a subject completely or partially lacking FXN expression), a baseline FXN(−) expression profile, a sample obtained from an FXN deficient subject following administration of FXN replacement therapy, or an FXN replacement expression profile.

A control sample can also be a sample from a subject from an earlier time point, e.g., prior to treatment with FXN replacement therapy. A control sample can be a purified sample, protein, and/or nucleic acid provided with a kit. Such control samples can be diluted, for example, in a dilution series to allow for quantitative measurement of levels of analytes, e.g., markers, in test samples. A control sample may include a sample derived from one or more subjects. A control sample may also be a sample taken at an earlier time point from the subject to be assessed. For example, the control sample could be a sample taken from the subject to be assessed before treatment with FXN replacement therapy. The control sample may also be a sample from an animal model, or from a tissue or cell line derived from the animal model of a mitochondrial disease such as FRDA. The level of activity or expression of one or more FSGMs (e.g., 1, 2, 3, 4, 5, 6, 7, 8, or 9 or more FSGMs) in a control sample consists of a group of measurements that may be determined, e.g., based on any appropriate statistical measurement, such as, for example, measures of central tendency including average, median, or modal values. In one embodiment, “different from a control” is preferably statistically significantly different from a control.

As used herein, “changed, altered, increased or decreased” is understood as having a level of the one or more FSGM to be detected at a level that is statistically different, e.g., increased or decreased, as compared to a control sample or to a predetermined threshold value e.g., from an FXN healthy subject (i.e., a subject with a normal FXN level), or a sample from an FXN deficient subject (i.e., a subject completely or partially lacking FXN expression). Changed, altered, increased or decreased, as compared to control or threshold value, can also include a difference in the rate of change of the level of one or more FSGMs obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of an FSGM in a sample versus a control, wherein the increase is above some threshold value, or a decrease in the detected level of an FSGM in a sample versus a control, wherein the decrease is below some threshold value.

As used herein, “detecting”, “detection”, “determining”, and the like are understood to refer to identification of the presence and/or level of one or more FSGMs selected from Table 1 and/or Table 5.

As used herein, the term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

As used herein, the terms “FXN deficient patient” or “FXN deficient subject”, used interchangeably herein, refer to a subject who has been determined to have an FXN deficiency, e.g., been diagnosed with Friedreich's Ataxia (FRDA). In some embodiments, an FXN deficient subject has a reduced level of FXN expression or activity, e.g., partially or completely lacking FXN expression or activity, as compared to a normal control subject (e.g., has less than 70%, 60%, 50%, 40%, 30%, 20%, or 10% of the FXN expression level or activity of a normal control subject). In some embodiments, an FXN deficient subject has not yet received FXN replacement therapy and is, therefore, therapy naive. In some embodiments, an FXN deficient subject is scheduled to receive FXN replacement therapy. In some embodiments, an FXN deficient subject is currently undergoing FXN replacement therapy. In some embodiments, an FXN deficient subject has already undergone an FXN replacement therapy. In some embodiments, an FXN deficient subject who is undergoing or has already undergone FXN replacement therapy has FXN levels that are partially or completely restored to FXN levels of a healthy subject.

Certain diseases result in FXN deficiencies in subjects, including mitochondrial diseases such as Friedreich's Ataxia (FRDA). As used herein, the term “FXN replacement therapy” refers to the introduction or replacement of frataxin in an FXN deficient subject which results in an increased level, expression and/or activity of frataxin in the subject. The FXN replacement therapy may result in an FXN level, expression or activity in the treated subject that is less than, e.g., some proportion of, that of a normal, healthy subject (e.g. partial replacement) or may result in a level, expression or activity of FXN in the treated subject that approaches or is approximately equal to that of a normal subject. The FXN replacement therapy may be carried out by delivery of any therapeutic agent capable of increasing a level, expression or activity of FXN in an FXN deficient subject. In some embodiments, FXN replacement therapy may be carried out through delivery of FXN protein or of a nucleic acid capable of increasing FXN levels or expression in an FXN deficient subject, e.g., DNA or mRNA encoding FXN, to a subject. Delivery of FXN protein to the subject can include delivery of FXN protein or delivery of an FXN fusion protein. In some embodiments, the nucleic acid capable of increasing FXN levels or activity in an FXN deficient subject is mRNA encoding FXN. In some embodiments, the nucleic acid capable of increasing FXN levels or activity in an FXN deficient subject is siRNA or an antisense oligonucleotide. In some embodiments, a nucleic acid capable of increasing FXN levels or activity in an FXN deficient subject may be delivered to a subject via a viral vector.

As used herein, the term “FXN fusion protein” refers to full length FXN or a fragment of FXN fused to a full length or a fragment of a different protein, or to a peptide. In some embodiments, an FXN fusion protein comprises full-length hFXN (SEQ ID NO: 1) or mature hFXN (SEQ ID NO: 2), as described herein. In some embodiments, the FXN protein or fragment thereof is fused to a cell penetrating peptide (CPP). In some embodiments, the CPP is an HW-TAT polypeptide. In some embodiments, FXN replacement therapy comprises administering to an FXN-deficient subject an FXN fusion protein comprising or consisting of SEQ ID NO: 12.

As used herein, the terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. An early stage disease state includes a state wherein one or more physical symptoms are not yet detectable. Certain characteristic signs, symptoms, and related factors can be quantitated through a variety of methods to yield important diagnostic information.

As used herein, the term “mitochondrial disease” refers to a disease which is the result of either inherited or spontaneous mutations in mtDNA or nDNA which leads to altered functions of the proteins or RNA molecules that normally reside in mitochondria, which decreases the functions of the mitochondria to induce diseases of various types in, for example, the central nervous system, skeletal muscles, heart, eyes, liver, kidneys, large intestine (colon), small intestine, internal ear and pancreas; as well as blood, skin and endocrine glands. In one non-limiting embodiment, the mitochondrial disease is Friedrich's Ataxis (FRDA).

As used herein, a sample obtained at an “earlier time point” is a sample that was obtained at a sufficient time in the past such that clinically relevant information could be obtained in the sample from the earlier time point as compared to the later time point. In certain embodiments, an earlier time point is at least 1, 2, 3, 4, 5, 6, 7, 8, 9, 10, 11, 12, 13, 14, 15, 16, 17, 18, 19, 20, 21, 22, or 23 hours, or 1, 2, 3, 4, 5, 6, or 7 days earlier. In some embodiments, an earlier time point is at least one, two, three or four weeks earlier. In certain embodiments, an earlier time point is at least six weeks earlier. In certain embodiments, an earlier time point is at least two months earlier. In certain embodiments, an earlier time point is at least three months earlier. In certain embodiments, an earlier time point is at least six months earlier. In certain embodiments, an earlier time point is at least nine months earlier. In certain embodiments, an earlier time point is at least one year earlier. Multiple subject samples (e.g., 3, 4, 5, 6, 7, or more) can be obtained at regular or irregular intervals over time and analyzed for trends in changes in FSGM levels. Appropriate intervals for testing for a particular subject can be determined by one of skill in the art based on ordinary considerations.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The term “expression profile” is used to include a genomic expression profile, meaning an expression profile of RNAs, or specifically of mRNAs or transcripts, or a protein expression profile. As used herein, expression profile may refer to a set of data obtained for mRNA expression. It may refer to the raw data in the readings of a PCR apparatus for example, or to the normalized expression values. Expression profiles may be determined by any convenient means for measuring a level of a nucleic acid sequence such as quantitative hybridization of mRNA, labeled mRNA, amplified mRNA, cDNA, etc., quantitative PCR, and other techniques known to a person skilled in the art or described herein. Expression profiles enable analysis of differential gene expression between two or more samples, between samples and control, as well as between samples and thresholds. An expression profile can also be determined by any means known to a person skilled in the art or described herein for measuring the level of a protein or a polypeptide, e.g., mass spectrometry, immunodetection assays, e.g., ELISA, etc.

As referred to herein, the term “FXN expression profile” includes any one of the following three FXN expression profiles: a normal FXN expression profile, a baseline FXN(−) expression profile, or an FXN replacement expression profile. In some embodiments, the baseline FXN(−) expression profile can be used as a control. In some embodiments, the normal FXN expression profile can be used as a control.

In some embodiments, the term “FXN expression profile” for one or more FSGMs refers to the expression level (e.g., RNA level or protein level) of the one or more FSGMs or to a value or set of values indicative of the level of expression of the one or more FSGMs. In some embodiments, an FXN expression profile comprises a feature vector of values indicative of expression of the one or more FSGMs. In some embodiments, “determining an FXN expression profile” for one or more FSGMs comprises detecting the expression level (e.g., RNA level or protein level) of the one or more FSGMs. In some embodiments, “determining an FXN expression profile” for one or more FSGMs comprises determining a feature vector of values indicative of the expression level (e.g., RNA level or protein level) of the one or more FSGMs.

As referred to herein, the term “normal FXN expression profile” refers to an expression profile of one or more FSGMs in a sample obtained from a normal, healthy subject or subjects (i.e., a subject that is not FXN deficient). In some embodiments, a “normal FXN expression profile” also encompasses an average of multiple, e.g., two or more, normal FXN expression profiles (e.g., from two or more subjects).

As referred to herein, the term “baseline FXN(−) expression profile” refers to the expression profile of one or more FSGMs in a sample from an FXN deficient subject prior to treatment with an FXN replacement therapy. In some embodiments, the term “baseline FXN(−) expression profile” encompasses an average of multiple, e.g., two or more, baseline FXN(−) expression profiles (e.g., from two more subjects).

An average FXN expression profile, e.g., an average normal FXN expression profile or an average baseline FXN(−) expression profile, may be determined by methods known in the art, e.g., by determining an expression level of one or more FSGMs in samples obtained from two or more subjects, e.g., normal subjects or FXN-deficient subjects, and then calculating an average expression level of the one or more FSGMs.

As referred to herein, the term “reference FXN expression profile” encompasses a “normal FXN expression profile” and a “baseline FXN(−) expression profile”, i.e., can be either one. The reference expression profile, e.g., reference normal FXN expression profile or reference baseline FXN(−) expression profile, can be used as a control, e.g., for comparing to an FXN replacement expression profile to evaluate response of a subject to an FXN replacement therapy.

As referred to herein, the term “FXN replacement expression profile” refers to the expression profile for one or more FSGMs in a sample obtained from an FXN-deficient subject subsequent to administration of at least one dose of an FXN replacement therapy.

As used herein, the term “treatment with FXN replacement therapy” refers to administration to a subject of at least one dose of an FXN replacement therapy. The terms “following treatment with FXN replacement therapy”, “subsequent to treatment with FXN replacement therapy”, “following administration of FXN replacement therapy” or “subsequent to administration of FXN replacement therapy”, as used herein, refers to at least 1 day, e.g., at least 2, 3, 4, 5, 6, 7, 8, 9, 10, 11, 12, 13, 14, 15, 16, 17, 18, 19, 20, 25, 30, 35, 40, or more days after administration of a dose of an FXN replacement therapy.

As referred to herein, the term “evaluate response of a subject to FXN replacement therapy” encompasses evaluating efficacy of FXN replacement therapy by (a) determining an FXN replacement expression profile in a sample from an FXN deficient subject following treatment with FXN replacement therapy; (b) comparing the FXN replacement expression profile with a reference FXN expression profile; and (c) using the comparison in step (b) to evaluate or determine efficacy of the FXN replacement therapy.

In some embodiments, the reference FXN expression profile is a baseline FXN(−) expression profile, i.e., the expression profile of one or more FSGMs in a sample obtained from an FXN deficient subject prior to treatment with FXN replacement therapy. In some embodiments, a difference between the FXN replacement expression profile and the baseline FXN(−) expression profile is indicative of the efficacy of or response to the FXN replacement therapy.

In some embodiments, the reference FXN expression profile is a normal FXN expression profile, i.e., the expression profile of one or more FSGMs in a sample obtained from a normal subject. In some embodiments, a comparison between the FXN replacement expression profile and the normal FXN expression profile, e.g., a similarity between the two profiles, is indicative of the efficacy of or response to the FXN replacement therapy.

A “higher level of expression”, “higher level”, “increased level,” and the like of an FSGM refers to an expression level in a test sample that is greater than the standard error of the assay employed to assess expression, and is preferably at least 25% more, at least 50% more, at least 75% more, at least two, at least three, at least four, at least five, at least six, at least seven, at least eight, at least nine, or at least ten times the expression level of the FSGM in a control sample (e.g., a sample from a healthy subject, a sample from an FXN deficient subject, or a sample from a subject following FXN replacement therapy) and preferably, the average expression level of the FSGM or FSGMs in several control samples.

As used herein, the term “hybridization,” as in “nucleic acid hybridization,” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra, or further in Higgins and Hames (Eds.) “Nucleic acid hybridization, a practical approach” IRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 2000, supra). Other protocols or commercially available hybridization kits (e.g., ExpressHyb® from BD Biosciences Clonetech) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

As used herein, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. In some embodiments, the term “identical” or “percent identity” refers to nucleic acid or amino acid sequences that do not comprise internal non-matching deletions or additions in the sequences relative to one another, i.e., the nucleic acid or amino acid sequences are of the same length and have a specified percentage of nucleotides or amino acid residues that are the same.

Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s).

The term “including” is used herein to mean, and is used interchangeably with, the phrase “including but not limited to.”

As used herein, a “label” refers to a molecular moiety or compound that can be detected or can lead to a detectable signal. A label is joined, directly or indirectly, to a molecule, such as an antibody, a nucleic acid probe or the protein/antigen or nucleic acid to be detected (e.g., an amplified sequence). Direct labeling can occur through bonds or interactions that link the label to the nucleic acid (e.g., covalent bonds or non-covalent interactions), whereas indirect labeling can occur through the use of a “linker” or bridging moiety, such as oligonucleotide(s) or small molecule carbon chains, which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Preferably, the label on a labeled probe is detectable in a homogeneous assay system, i.e., in a mixture, the bound label exhibits a detectable change compared to an unbound label.

The terms “level of expression of a gene”, “gene expression level”, “level of an FSGM”, and the like refer to the level of mRNA, as well as pre-mRNA nascent transcript(s), transcript processing intermediates, mature mRNA(s) and degradation products, or the level of protein, encoded by the gene in the cell. The “level” of one or more FSGMs means the absolute or relative amount or concentration of the FSGM in the sample.

A “lower level of expression” or “lower level” or “decreased level” and the like of an FSGM refers to an expression level in a test sample that is less than 90%, 85%, 80%, 75%, 70%, 65%, 60%, 55%, 50%, 45%, 40%, 35%, 30%, 25%, 20%, 15%, or 10% of the expression level of the FSGM in a control sample (e.g., a sample from a healthy subject, a sample from an FXN deficient subject, or a sample from a subject following FXN replacement therapy) and preferably, the average expression level of the FSGM in several control samples.

As used herein, “nucleic acid molecule” or “polynucleotides”, refers to a polymer of nucleotides, including an FSGM. Non-limiting examples thereof include DNA (e.g., genomic DNA, cDNA), RNA molecules (e.g., mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Intl Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criterion like stability for example. Chimeras of deoxyribonucleotides and ribonucleotides may also be within the scope of the present invention.

As used herein, “one or more” is understood as encompassing each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise.

As used herein, “patient” or “subject” can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient.

As used herein, the term “progression of FXN deficiency” refers to worsening of a condition of an FXN deficient subject over time. This term encompasses an increase in severity and/or duration of existing symptoms of FXN deficiency and/or appearance of one or more new symptoms of FXN deficiency in an FXN deficient subject. In some embodiments, progression of FXN deficiency is correlated with levels of FXN in an FXN deficient subject. For example, in some embodiments, progression of FXN deficiency over time is associated with a decreased level of FXN over time.

As used herein, a “probe” is meant to include a nucleic acid oligomer or oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. Numerous primers and probes which can be designed and used in the context of the present invention can be readily determined by a person of ordinary skill in the art to which the present invention pertains.

As used herein, a “reference level” of an FSGM may be an absolute or relative amount or concentration of the FSGM, a presence or absence of the FSGM, a range of amount or concentration of the FSGM, a minimum and/or maximum amount or concentration of the FSGM, a mean amount or concentration of the FSGM, and/or a median amount or concentration of the FSGM; and, in addition, “reference levels” of combinations of FSGMs may also be ratios of absolute or relative amounts or concentrations of two or more FSGMs with respect to each other. Appropriate positive and negative reference levels of FSGMs for a particular disease state, phenotype, or lack thereof may be determined by measuring levels of desired FSGMs in one or more appropriate subjects, and such reference levels may be tailored to specific populations of subjects (e.g., a reference level may be age-matched so that comparisons may be made between FSGM levels in samples from subjects of a certain age and reference levels for a particular disease state, phenotype, or lack thereof in a certain age group). Such reference levels may also be tailored to specific techniques that are used to measure levels of FSGMs in biological samples (e.g., LC-MS, GC-MS, etc.), where the levels of FSGMs may differ based on the specific technique that is used.

As used herein, “sample” or “biological sample” includes a specimen or culture obtained from any source. In some embodiments, a sample includes any specimen or culture that comprises cells in which FXN expression profile may be analyzed. In some embodiments, a sample includes any specimen or culture from a subject deficient in FXN or a subject being treated with FXN replacement therapy. For example, biological samples can be obtained from a solid tissue sample, preferably a buccal sample, alternatively a skin biopsy sample, skin strip, hair follicle, muscle biopsy sample, or a body fluid sample such as blood (including any blood product, such as whole blood, plasma, serum, or specific types of cells of the blood), urine, saliva, or seminal fluid. In one embodiment, the biological sample is a buccal sample. In another embodiment, the biological sample is a skin sample, e.g., a skin biopsy sample or a skin strip. Alternatively, a sample can comprise exosomes which may be harvested in order to be tested for FSGMs transcripts.

As use herein, the phrase “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

The term “such as” is used herein to mean, and is used interchangeably, with the phrase “such as but not limited to.”

A “transcribed polynucleotide” or “nucleotide transcript” is a polynucleotide (e.g. an mRNA, hnRNA, a cDNA, or an analog of such RNA or cDNA) which is complementary to or having a high percentage of identity (e.g., at least 80% identity) with all or a portion of a mature mRNA made by transcription of an FSGM of the invention and normal post-transcriptional processing (e.g. splicing), if any, of the RNA transcript, and reverse transcription of the RNA transcript.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### C. FXN-Sensitive Genomic Markers (FSGMs) of the Invention

The present disclosure provides a set of markers, also referred to herein as FXN-sensitive genomic markers (or FSGMs), whose respective levels are positively or negatively correlated to frataxin (FXN) levels in a cell or a subject (e.g., a sample from a subject). In some embodiments, the FSGMs of the present disclosure are contrary regulated by FXN gene ablation or deficiency, followed by FXN protein replacement. Thus, said FSGMs of the present disclosure are both associated with FXN deficiency in a subject and conversely associated with FXN replacement. All of the FSGMs disclosed herein were found to be sensitive to FXN levels and are considered markers of FXN replacement therapy. Therefore, these FSGMs can be used to evaluate and/or monitor progression of FXN deficiency in a subject, as described herein. These FSGMs can also be used to evaluate and/or monitor an FXN replacement therapy, e.g., evaluate, monitor, or determine efficacy of an FXN replacement therapy, in a subject, as described herein.

For example, in one aspect, the present invention provides a method for determining, evaluating, and/or monitoring the effectiveness of FXN replacement therapy comprising determining: (i) a baseline FXN(−) expression profile for one or more FSGMs in a sample from an FXN deficient subject prior to treatment with FXN replacement therapy; and (ii) determining a subject FXN replacement expression profile for the one or more FSGMs in a sample from an FXN deficient subject undergoing FXN replacement therapy or subsequent to treatment with FXN replacement therapy; comparing the subject FXN replacement expression profile with the baseline FXN(−) expression profile; and using the results of the comparison to determine, evaluate, or monitor effectiveness of the FXN replacement therapy. Based on the results of the FSGM expression profile analysis, adjustments can be made to the FXN replacement therapy in the subject to, e.g., initiate, increase (e.g., increase dose and/or frequency of administration), decrease (e.g., decrease dose and/or frequency of administration), or cease FXN replacement therapy in the subject.

Another aspect of the disclosure relates to a method for identifying one or more FSGMs. The method comprises determining the expression profile of one or more genes in a sample from a healthy subject, having normal FXN levels, referred to herein as a normal FXN expression profile; determining the expression profile of the one or more genes in a sample from a subject having deficient FXN levels, referred to herein as a baseline FXN(−) expression profile; and comparing the normal FXN expression profile with the baseline FXN(−) expression profile; wherein the markers whose expression is altered in the baseline FXN(−) expression profile compared to the normal FXN expression profile are identified as FSGMs. Additionally, or alternatively, the method for determining FSGMs may comprise a comparison between the expression profiles of one or more genes obtained from a sample from an FXN deficient subject before receiving FXN replacement therapy (baseline FXN(−) expression profile) and the expression profiles of the one or more genes obtained from a sample from the FXN deficient subject during or after receiving FXN replacement therapy. The gene expression profile from a sample obtained from an FXN deficient subject during or after FXN replacement therapy is also referred to herein as an FXN replacement expression profile. In embodiments, the markers whose expression is altered in the FXN replacement expression profile as compared to the baseline FXN expression profile, e.g., altered towards, or similar to, levels of that of a normal FXN expression profile, are identified as FSGMs. By way of example, Table 1 herein presents FSGMs that were identified by a method of an embodiment of the disclosure.

TABLE 1
FXN-sensitive genomic markers (FSGMs) of the disclosure

| | Log2 fold change in | | NCBI Accession |
|---|---|---|---|
| | expression level healthy | BH adjusted | No. for mRNA |
| Gene | vs. FRDA subjects | p-value | sequence |
| LY96 | 4.92 | 1.14E−06 | NM_015364.2 |
| LRRK2 | 5.14 | 1.13E−05 | NM_198578.3 |
| Adgre1 | 4.11 | 1.13E−05 | NM_001974.3 |
| Timp1 | 3.29 | 1.13E−05 | NM_003254.2 |
| Xpo6 | 4.14 | 1.45E−05 | NM_015171.2 |
| CD44 | 3.64 | 1.38E−05 | NM_001001392.1 |
| BASP1 | 2.02 | 1.23E−05 | NM_006317.3 |
| PSMB8 | 2.86 | 0.000188 | NM_004159.4 |
| C3ar1 | 3.26 | 0.000265 | NM_004054.2 |
| TFRC | 3.62 | 0.000319 | NM_001128148.3 |
| SOD2 | 2.33 | 0.000319 | NM_000636.2 |
| PLCL2 | 2.38 | 0.000436 | NM_015184.5 |
| S100a4 | 1.38 | 0.000301 | NM_002961.2 |
| Mapre1 | 1.55 | 0.000327 | NM_012325.2 |
| PSMB9 | 1.8 | 0.000984 | NM_002800.4 |
| Lgals3 | −1.34 | 0.000142 | NM_001177388.1 |
| Ube2v2 | −1.1 | 0.000223 | NM_003350.2 |
| Sec61g | −1.92 | 0.00132 | NM_001012456.1 |
| Zdhhc13 | −2.11 | 0.00246 | NM_019028.2 |
| Yipf5 | −1.71 | 0.00373 | NM_001024947.3 |
| Tpp2 | −2.15 | 0.0038 | NM_003291.2 |
| CFH | 1.64 | 0.0104 | NM_001014975.2 |
| Tmem70 | 1.78 | 0.0162 | NM_017866.5 |
| Ybx2 | −2.06 | 0.0218 | NM_015982.3 |
| Ptms | −1 | 0.0307 | NM_002824.4 |
| FTL | −1.32 | 0.0078 | NM_000146.3 |
| TXNIP | −2.29 | 0.000345 | NM_006472.3 |

The FSGMs of the invention include, but are not limited to, any one or any combination of two or more of the FSGMs listed in Table 1 and/or Table 5. In some embodiments, the FSGMs of the invention include any one or any combination of two or more of the FSMGS listed in Table 1. In some embodiments, the FSGMs of the invention include any one or any combination of two or more of the FSMGS listed in Table 5. In some embodiments, the FSGMs of the invention include any one or any combination of two or more of the FSMGS listed in Table 1 and/or Table 5 in combination with one or more additional FSGMs listed in Tables 2, 3, 4 and/or 6. In some embodiments, the FSGMs of the invention include any one or any combination of two or more of the FSMGS listed in Table 1 in combination with one or more additional FSGMs listed in Tables 2, 3, 4 and/or 6. In some embodiments, the FSGMs of the invention include any one or any combination of two or more of the FSMGS listed in Table 5 in combination with one or more additional FSGMs listed in Tables 2, 3, 4 and/or 6. It will be recognized that in some embodiments of the present invention, other markers known in the art for measuring FXN expression levels or evaluating efficacy of FXN replacement therapy can be used in connection with the FSGMs and/or the methods of the present invention.

As used herein, the term “one or more FSGMs” is intended to mean that one or more (e.g., 1, 2, 3, 4, 5, 6, 7, 8, 9, 10, or more) FSGMs is selected, e.g., from Table 1 and/or Table 5. In some embodiments, one or more (e.g., 1, 2, 3, 4, 5, 6, 7, 8, 9, 10, or more) FSGMs is selected from Table 1. In some embodiments, one or more (e.g., 1, 2, 3, 4, 5, 6, 7, 8, 9, 10, or more) FSGMs is selected from Table 5. In some embodiments, one or more (e.g., 1, 2, 3, 4, 5, 6, 7, 8, 9, 10, or more) FSGMs is selected, e.g., from Table 1 and/or Table 5, alone or in combination with one or more (e.g., 1, 2, 3, 4, 5, 6, 7, 8, 9, 10, or more) additional FSGMs listed in Tables 2, 3, 4 and/or 6. Methods, kits, and panels provided herein include one or any combination of, e.g., 1, 2, 3, 4, 5, 6, 7, 8, 9, 10, or more FSGMs selected from Table 1 and/or Table 5. In some embodiments, methods, kits, and panels provided herein include one or any combination of, e.g., 1, 2, 3, 4, 5, 6, 7, 8, 9, 10, or more FSGMs selected from Table 1 and/or Table 5, alone or in combination with one or more additional FSGMs listed in Tables 2, 3, 4 and/or 6.

In some embodiments, the one or more FSGMs of the invention comprise one or more (e.g., 1, 2, 3, 4, 5, 6, 7, 8, 9, 10, or more) markers selected from Table 1 and/or Table 5, alone or in combination with one or more additional FSGMs selected from Tables 2, 3, 4 and/or 6.

In some embodiments, the one or more FSGMs comprise one or any combination of Timp1, LRRK2, BASP1, CD44, Xpo6, Lgals3, SOD2, PSMB9, FTL and TXNIP. In some embodiments, the one or more FSGMs comprise one or any combination of Timp1, LRRK2, BASP1, CD44, Xpo6, Lgals3, SOD2, PSMB9, FTL and TXNIP in combination with one or more additional FSGMs selected from Tables 2, 3, 4 and/or 6. In other embodiments, the one or more FSGMs comprise one or any combination of Timp1, LRRK2, BASP1, CD44, Xpo6, Lgals3 and PSMB9. In other embodiments, the one or more FSGMs comprise one or any combination of Timp1, LRRK2, BASP1, CD44, Xpo6, Lgals3 and PSMB9 in combination with one or more additional FSGMs selected from Tables 2, 3, 4 and/or 6. In some embodiments, the one or more FSGMs comprise one or any combination of Timp1, LRRK2, BASP1, CD44, Xpo6, Lgals3, PSMB9, FTL and TXNIP. In other embodiments, the one or more FSGMs comprise one or any combination of Timp1, LRRK2, BASP1, CD44, Xpo6, Lgals3, PSMB9, FTL and XNIP in combination with one or more additional FSGMs selected from Tables 2, 3, 4 and/or 6.

In one embodiment, the one or more FSGMs comprise one or any combination of LRRK2, SOD2, CD44, and Lgals3, alone or in combination with one or more additional FSGM selected from Table 4 and/or Table 5. In another embodiment, the one or more FSGMs comprise one or any combination of LRRK2, CD44, and Lgals3, alone or in combination with one or more additional FSGM selected from Tables 2, 3, 4 and/or 6. In one embodiment, the one or more FSGMs comprise one or any combination of LRRK2, SOD2, CD44, Lgals3, FTL and TXNIP, alone or in combination with one or more additional FSGM selected from Table 4 and/or Table 5. In another embodiment, the one or more FSGMs comprise one or any combination of LRRK2, CD44, Lgals3, FTL and TXNIP, alone or in combination with one or more additional FSGM selected from Tables 2, 3, 4 and/or 6.

In one embodiment, the one or more FSGMs comprise one or any combination of Timp1, LRRK2, BASP1, CD44, Xpo6, Lgals3, SOD2 and PSMB9, alone or in combination with one or any combination of CTSS, BTG2, EGR1 and PTGS2. In another embodiment, the one or more FSGMs comprise one or any combination of Timp1, LRRK2, BASP1, CD44, Xpo6, Lgals3 and PSMB9, alone or in combination with one or any combination of CTSS, BTG2, EGR1 and PTGS2.

In one embodiment, the one or more FSGMs comprise one or any combination of Timp1, LRRK2, BASP1, CD44, Xpo6, Lgals3, SOD2, PSMB9, FTL, TXNIP, alone or in combination with one or any combination of CTSS, BTG2, EGR1 and PTGS2. In another embodiment, the one or more FSGMs comprise one or any combination of Timp1, LRRK2, BASP1, CD44, Xpo6, Lgals3, PSMB9, FTL, TXNIP, alone or in combination with one or any combination of CTSS, BTG2, EGR1 and PTGS2.

In one embodiment, the one or more FSGMs comprise one or any combination of LRRK2, SOD2, CD44 and Lgals3, alone or in combination with one or any combination of CTSS, BTG2, EGR1 and PTGS2. In another embodiment, the one or more FSGMs comprise one or any combination of LRRK2, CD44 and Lgals3, alone or in combination with one or any combination of CTSS, BTG2, EGR1 and PTGS2.

In one embodiment, the one or more FSGMs comprise one or any combination of LRRK2, SOD2, CD44, Lgals3, FTL and TXNIP, alone or in combination with one or any combination of CTSS, BTG2, EGR1 and PTGS2. In another embodiment, the one or more FSGMs comprise one or any combination of LRRK2, CD44, Lgals3, FTL and TXNIP, alone or in combination with one or any combination of CTSS, BTG2, EGR1 and PTGS2.

In one embodiment, the one or more FSGMs comprise Xpo6, SOD2, CTSS, EGR1, BTG2, PTSG2 and LRKK2. In one embodiment, the one or more FSGMs comprise Xpo6, SOD2, FTL, TXNIP, CTSS, EGR1, BTG2, PTSG2 and LRKK2.

The additional FSGMs that are useful in the context of the present disclosure are listed in Table 2 and Table 3 below.

TABLE 2
Additional FSGMs of the Disclosure
Gene Symbol

Abce1
Adamts1
Adnp
AI480526
Apold1
Arc
Aspn
Atf3
Bicd1
Btg2
C230034O21Rik
Calm2
Capza1
Ccdc85b
Ccdc85c
Chm
Cops2
Cript
Ctcfl
Ctss
Cul2
Cycs
Cyr61
D130020L05Rik
Dclk1
Dcun1d1
Dfna5
Dio2
Dnajb9
Dsel
Dynlt3
Egr1
Egr2
Egr3
Eif1a
EIF1AX
Emp1
Fam177a
FAM177A1
(C14ORF24)
Gmfb
H4C13
Hist1h4n
Igf1
Kctd12b
Lamp2
Lamtor5
Lox
Lypla1
Lysmd3
Maoa
Mki67
Mob4
Mpeg1
Mt2
mt-Atp6
mt-Atp8
mt-CO2
mt-CO3
mt-Nd1
mt-Nd2
mt-Nd3
mt-Nd4
mt-Rnr1
mt-Rnr2
Nr4a1
Nrtn
Orc4
Pde4a
Pde4b
Phf1
Psma3
Ptgs2
Ptp4a1
Ptprc
Rap1b
Rap2c
Rnf13
Rnf2
Rpl10
Rpl24
Rpl26
Rpl32
Rpl37rt
Rpl38
Rpl39
Rps15a
Rps23
Rps27l
Rtn4
Serpine1
Slirp
Snord17
Spry4
Stc1
Suv420h2
Thbs1
Tmem126a
Top2a
Ube2d3
Vbp1
Wnk2
Yam1
Yars
Zfp758
ZNF300
ZNF34
Znf41-ps
Znrf1

TABLE 3
Exemplary GenBank Accession Numbers Corresponding
to the Additional FSGMs of the Disclosure

| | NCBI Accession | NCBI Accession |
|---|---|---|
| Gene | No. for mRNA | No. for Protein |
| Symbol | sequence | sequence |
| Abce1 | NM_001040876.2 | NP_001035809.1 |
| Adamts1 | NM_006988.5 | NP_008919.3 |
| Adnp | NM_001282531.3 | NP_001269460.1 |
| Apold1 | NM_001130415.2 | NP_001123887.1 |
| Arc | NM_015193.5 | NP_056008.1 |
| Aspn | NM_001193335.2 | NP_001180264.1 |
| Atf3 | NM_001030287.3 | NP_001025458.1 |
| Bicd1 | NM_001003398.3 | NP_001003398.1 |
| Btg2 | NM_006763.3 | NP_006754.1 |
| Calm2 | NM_001305624.1 | NP_001292553.1 |
| Capza1 | NM_006135.3 | NP_006126.1 |
| Ccdc85b | NM_006848.3 | NP_006839.2 |
| Ccdc85c | NM_001144995.2 | NP_001138467.1 |
| Chm | NM_000390.4 | NP_000381.1 |
| Cops2 | NM_001143887.2 | NP_001137359.1 |
| Cript | NM_014171.6 | NP_054890.1 |
| Ctcfl | NM_001269040.1 | NP_001255969.1 |
| Ctss | NM_001199739.2 | NP_001186668.1 |
| Cul2 | NM_001198777.2 | NP_001185706.1 |
| Cycs | NM_018947.6 | NP_061820.1 |
| Cyr61 | NM_001554.5 | NP_001545.2 |
| Delk1 | NM_001195415.1 | NP_001182344.1 |
| Dcun1d1 | NM_001308101.2 | NP_001295030.1 |
| Dfna5 | NM_001127453.2 | NP_001120925.1 |
| Dio2 | NM_000793.6 | NP_000784.3 |
| Dnajb9 | NM_012328.3 | NP_036460.1 |
| Dsel | NM_032160.3 | NP_115536.2 |
| Dynlt3 | NM_006520.3 | NP_006511.1 |
| Egr1 | NM_001964.3 | NP_001955.1 |
| Egr2 | NM_000399.5 | NP_000390.2 |
| Egr3 | NM_001199880.2 | NP_001186809.1 |
| EIF1AX | NM_001412.4 | NP_001403.1 |
| Emp1 | NM_001423.3 | NP_001414.1 |
| FAM177A1(C14ORF24) | NM_001079519.1 | NP_001072987.1 |
| | NM_001289022.2 | NP_001275951.1 |
| | NM_173607.4 | NP_775878.2 |
| Gmfb | NM_004124.3 | NP_004115.1 |
| H4C13 | NM_003546.3 | NP_003537.1 |
| Igf1 | M_000618.5 | NP_000609.1 |
| KCTD12 | NM_138444.4 | NP_612453.1 |
| Lamp2 | NM_001122606.1 | NP_001116078.1 |
| Lamtor5 | NM_006402.2 | NP_006393.2 |
| Lox | NM_001178102.2 | NP_001171573.1 |
| Lypla1 | NM_001279356.1 | NP_001266285.1 |
| Lysmd3 | NM_001286812.1 | NP_001273741.1 |
| Maoa | NM_000240.4 | NP_000231.1 |
| Mki67 | NM_001145966.2 | NP_001139438.1 |
| Mob4 | NM_001100819.3 | NP_001094289.1 |
| Mpeg1 | NM_001039396.2 | NP_001034485.1 |
| Mt2a | NM_005953 | NP_005944 |
| mt-Atp6 | J01415 | YP_003024031.1 |
| mt-Atp8 | J01415 | YP_003024030.1 |
| mt-Co3 | J01415 | YP_003024032.1 |
| mt-Nd1 | J01415 | YP_003024026.1 |
| mt-Nd2 | J01415 | YP_003024027.1 |
| mt-Nd3 | J01415 | YP_003024033.1 |
| mt-Nd4 | J01415 | YP_003024035.1 |
| mt-Rnr1 | J01415 | |
| mt-Rnr2 | J01415 | |
| Nr4a1 | NM_001202233.1 | NP_001189162.1 |
| Nrtn | NM_004558.4 | NP_004549.1 |
| Orc4 | NM_001190879.2 | NP_001177808.1 |
| Pde4a | NM_001111307.2 | NP_001104777.1 |
| Pde4b | NM_001037339.2 | NP_001032416.1 |
| Phf1 | NM_002636.5 | NP_002627.2 |
| Psma3 | NM_002788.4 | NP_002779.1 |
| Ptgs2 | NM_000963.4 | NP_000954.1 |
| Ptp4a1 | NM_003463.4 | NP_003454.1 |
| Ptprc | NM_001267798.2 | NP_001254727.1 |
| Rap1b | NM_001010942.3 | NP_001010942.1 |
| Rap2c | NM_001271186.2 | NP_001258115.1 |
| Rnf13 | NM_001378285.1 | NP_001365214.1 |
| Rnf2 | NM_007212.4 | NP_009143.1 |
| Rpl10 | NM_001256577.2 | NP_001243506.2 |
| Rpl24 | NM_000986.4 | NP_000977.1 |
| Rpl26 | NM_000987.5 | NP_000978.1 |
| Rpl32 | NM_000994.4 | NP_000985.1 |
| Rpl38 | NM_000999.4 | NP_000990.1 |
| Rpl39 | NM_001000.4 | NP_000991.1 |
| Rps15a | NM_001019.5 | NP_001010.2 |
| Rps271 | NM_015920.4 | NP_057004.1 |
| Rtn4 | NM_001321859.2 | NP_001308788.1 |
| Serpine1 | NM_000602.5 | NP_000593.1 |
| Slirp | NM_001267863.1 | NP_001254792.1 |
| Spry4 | NM_001127496.3 | NP_001120968.1 |
| Stc1 | NM_003155.3 | NP_003146.1 |
| Suv420h2 | NM_032701.4 | NP_116090.2 |
| Thbs1 | NM_003246.4 | NP_003237.2 |
| Tmem126a | NM_001244735.1 | NP_001231664.1 |
| Top2a | NM_001067.4 | NP_001058.2 |
| Ube2d3 | NM_001300795.1 | NP_001287724.1 |
| Vbp1 | NM_001303543.1 | NP_001290472.1 |
| Wnk2 | NM_001282394.1 | NP_001269323.1 |
| Yam1 | | N/A |
| Yars | NM_003680.3 | NP_003671.1 |
| ZNF34 | NM_001286769.2 | NP_001273698.1 |
| | NM_001286770.1 | NP_001273699.1 |
| | NM_001378027.1 | NP_001364956.1 |
| | NM_001378028.1 | NP_001364957.1 |
| | NM_001378029.1 | NP_001364958.1 |
| | NM_030580.4 | NP_085057.3 |
| ZNF300 | NM_001172831.1 | NP_001166302.1 |
| | NM_001172832.1 | NP_001166303.1 |
| | NM_052860.2 | NP_443092.1 |
| Znrf1 | NM_032268.5 | NP_115644.1 |

In one embodiment, the one or more FSGMs comprise one or more of Timp1, LRRK2, BASP1, CD44, Xpo6, Lgals3, SOD2, PSMB9, FTL and TXNIP. In another embodiment, the one or more FSGMs comprise two or more of Timp1, LRRK2, BASP1, CD44, Xpo6, Lgals3, SOD2, PSMB9, FTL and TXNIP. In another embodiment, the one or more FSGMs comprise three or more of Timp1, LRRK2, BASP1, CD44, Xpo6, Lgals3, SOD2, PSMB9, FTL and TXNIP. In another embodiment, the one or more FSGMs comprise four or more of Timp1, LRRK2, BASP1, CD44, Xpo6, Lgals3, SOD2, PSMB9, FTL and TXNIP. In another embodiment, the one or more FSGMs comprise five or more of Timp1, LRRK2, BASP1, CD44, Xpo6, Lgals3, SOD2, PSMB9, FTL and TXNIP. In another embodiment, the one or more FSGMs comprise six or more of Timp1, LRRK2, BASP1, CD44, Xpo6, Lgals3, SOD2, PSMB9, FTL and TXNIP. In another embodiment, the one or more FSGMs comprise seven or more of Timp1, LRRK2, BASP1, CD44, Xpo6, Lgals3, SOD2, PSMB9, FTL and TXNIP. In another embodiment, the one or more FSGMs comprise eight or more of Timp1, LRRK2, BASP1, CD44, Xpo6, Lgals3, SOD2, PSMB9, FTL and TXNIP. In another embodiment, the one or more FSGMs comprise nine or more of Timp1, LRRK2, BASP1, CD44, Xpo6, Lgals3, SOD2, PSMB9, FTL and TXNIP. In another embodiment, the one or more FSGMs comprise Timp1, LRRK2, BASP1, CD44, Xpo6, Lgals3, SOD2, PSMB9, FTL and TXNIP.

In one embodiment, the one or more FSGMs comprise one or more of Timp1, LRRK2, BASP1, CD44, Xpo6, Lgals3, PSMB9, FTL and TXNIP. In another embodiment, the one or more FSGMs comprise two or more of Timp1, LRRK2, BASP1, CD44, Xpo6, Lgals3, PSMB9, FTL and TXNIP. In another embodiment, the one or more FSGMs comprise three or more of Timp1, LRRK2, BASP1, CD44, Xpo6, Lgals3, PSMB9, FTL and TXNIP. In another embodiment, the one or more FSGMs comprise four or more of Timp1, LRRK2, BASP1, CD44, Xpo6, Lgals3, PSMB9, FTL and TXNIP. In another embodiment, the one or more FSGMs comprise five or more of Timp1, LRRK2, BASP1, CD44, Xpo6, Lgals3, PSMB9, FTL and TXNIP. In another embodiment, the one or more FSGMs comprise six or more of Timp1, LRRK2, BASP1, CD44, Xpo6, Lgals3, PSMB9, FTL and TXNIP. In another embodiment, the one or more FSGMs comprise seven or more of Timp1, LRRK2, BASP1, CD44, Xpo6, Lgals3, PSMB9, FTL and TXNIP. In another embodiment, the one or more FSGMs comprise eight or more of Timp1, LRRK2, BASP1, CD44, Xpo6, Lgals3, PSMB9, FTL and TXNIP. In another embodiment, the one or more FSGMs comprise Timp1, LRRK2, BASP1, CD44, Xpo6, Lgals3, PSMB9, FTL and TXNIP.

In some embodiments, the one or more FSGMs comprise Timp 1. In some embodiments, the one or more FSGMs comprise LRRK2. In some embodiments, the one or more FSGMs comprise BASP1. In some embodiments, the one or more FSGMs comprise CD44. In some embodiments, the one or more FSGMs comprise Xpo6. In some embodiments, the one or more FSGMs comprise Lgals3. In some embodiments, the one or more FSGMs comprise SOD2. In some embodiments, the one or more FSGMs comprise PSMB9. In some embodiments, the one or more FSGMs comprise FTL. In some embodiments, the one or more FSGMs comprise TXNIP.

In some embodiments, the one or more FSGMs consist of Timp 1. In some embodiments, the one or more FSGMs consist of LRRK2. In some embodiments, the one or more FSGMs consist of BASP1. In some embodiments, the one or more FSGMs consist of CD44. In some embodiments, the one or more FSGMs consist of Xpo6. In some embodiments, the one or more FSGMs consist of Lgals3. In some embodiments, the one or more FSGMs consist of SOD2. In some embodiments, the one or more FSGMs consist of PSMB9. In some embodiments, the one or more FSGMs consist of FTL. In some embodiments, the one or more FSGMs consist of TXNIP.

In some embodiments, the one or more FSGMs comprise Timp 1 and LRRK2. In some embodiments, the one or more FSGMs comprise Timp 1 and Basp1. In some embodiments, the one or more FSGMs comprise Timp 1 and CD44. In some embodiments, the one or more FSGMs comprise Timp 1 and Xpo6. In some embodiments, the one or more FSGMs comprise Timp 1 and Lgals3. In some embodiments, the one or more FSGMs comprise Timp 1 and SOD2. In some embodiments, the one or more FSGMs comprise Timp 1 and PSMB9. In some embodiments, the one or more FSGMs comprise Timp 1 and FTL. In some embodiments, the one or more FSGMs comprise Timp 1 and TXNIP. In some embodiments, the one or more FSGMs comprise LRRK2 and BASP1. In some embodiments, the one or more FSGMs comprise LRRK2 and CD44. In some embodiments, the one or more FSGMs comprise LRRK2 and Xpo6. In some embodiments, the one or more FSGMs comprise LRRK2 and Lgals3. In some embodiments, the one or more FSGMs comprise LRRK2 and SOD2. In some embodiments, the one or more FSGMs comprise LRRK2 and PSMB9. In some embodiments, the one or more FSGMs comprise LRRK2 and FTL. In some embodiments, the one or more FSGMs comprise LRRK2 and TXNIP. In some embodiments, the one or more FSGMs comprise BASP1 and CD44. In some embodiments, the one or more FSGMs comprise BASP1 and Xpo6. In some embodiments, the one or more FSGMs comprise BASP1 and Lgals3. In some embodiments, the one or more FSGMs comprise BASP1 and SOD2. In some embodiments, the one or more FSGMs comprise BASP1 and PSMB9. In some embodiments, the one or more FSGMs comprise BASP1 and FTL. In some embodiments, the one or more FSGMs comprise BASP1 and TXNIP. In some embodiments, the one or more FSGMs comprise CD44 and Xpo6. In some embodiments, the one or more FSGMs comprise CD44 and Lgals3. In some embodiments, the one or more FSGMs comprise CD44 and SOD2. In some embodiments, the one or more FSGMs comprise CD44 and PSMB9. In some embodiments, the one or more FSGMs comprise CD44 and FTL. In some embodiments, the one or more FSGMs comprise CD44 and TXNIP. In some embodiments, the one or more FSGMs comprise Xpo6 and Lgals3. In some embodiments, the one or more FSGMs comprise Xpo6 and SOD2. In some embodiments, the one or more FSGMs comprise Lgals3 and PSMB9. In some embodiments, the one or more FSGMs comprise Lgals3 and FTL. In some embodiments, the one or more FSGMs comprise Lgals3 and TXNIP. In some embodiments, the one or more FSGMs comprise Lgals3 and SOD2. In some embodiments, the one or more FSGMs comprise Lgals3 and PSMB9. In some embodiments, the one or more FSGMs comprise SOD2 and PSMB9. In some embodiments, the one or more FSGMs comprise SOD2 and FTL. In some embodiments, the one or more FSGMs comprise SOD2 and TXNIP.

In some embodiments, the one or more FSGMs comprise Timp1, LRRK2 and BASP1. In some embodiments, the one or more FSGMs comprise Timp1, LRRK2 and CD44. In some embodiments, the one or more FSGMs comprise Timp1, LRRK2 and Xpo6. In some embodiments, the one or more FSGMs comprise Timp1, LRRK2 and Lgals3. In some embodiments, the one or more FSGMs comprise Timp1, LRRK2 and SOD2. In some embodiments, the one or more FSGMs comprise Timp1, LRRK2 and PSMB9. In some embodiments, the one or more FSGMs comprise Timp1, LRRK2 and FTL. In some embodiments, the one or more FSGMs comprise Timp1, LRRK2 and FXNIP. In some embodiments, the one or more FSGMs comprise LRRK2, BASP1 and CD44. In some embodiments, the one or more FSGMs comprise LRRK2, BASP1 and Xpo6. In some embodiments, the one or more FSGMs comprise LRRK2, BASP1 and Lgals3. In some embodiments, the one or more FSGMs comprise LRRK2, BASP1 and SOD2. In some embodiments, the one or more FSGMs comprise LRRK2, BASP1 and PSMB9. In some embodiments, the one or more FSGMs comprise LRRK2, BASP1 and FTL. In some embodiments, the one or more FSGMs comprise LRRK2, BASP1 and TXNIP. In some embodiments, the one or more FSGMs comprise BASP1, CD44 and Xpo6. In some embodiments, the one or more FSGMs comprise BASP1, CD44 and Lgals3. In some embodiments, the one or more FSGMs comprise BASP1, CD44 and SOD2. In some embodiments, the one or more FSGMs comprise BASP1, CD44 and PSMB9. In some embodiments, the one or more FSGMs comprise BASP1, CD44 and FTL. In some embodiments, the one or more FSGMs comprise BASP1, CD44 and TXNIP. In some embodiments, the one or more FSGMs comprise CD44, Xpo6 and Lgals3. In some embodiments, the one or more FSGMs comprise CD44, Xpo6 and SOD2. In some embodiments, the one or more FSGMs comprise CD44, Xpo6 and PSMB9. In some embodiments, the one or more FSGMs comprise CD44, Xpo6 and FTL. In some embodiments, the one or more FSGMs comprise CD44, Xpo6 and TXNIP. In some embodiments, the one or more FSGMs comprise Xpo6, Lgals3 and SOD2. In some embodiments, the one or more FSGMs comprise Xpo6, Lgals3 and PSMB9. In some embodiments, the one or more FSGMs comprise Xpo6, Lgals3 and FTL. In some embodiments, the one or more FSGMs comprise Xpo6, Lgals3 and TXNIP. In some embodiments, the one or more FSGMs comprise Timp1, BASP1 and CD44. In some embodiments, the one or more FSGMs comprise Timp1, BASP1 and Xpo6. In some embodiments, the one or more FSGMs comprise Timp1, BASP1 and Lgals3. In some embodiments, the one or more FSGMs comprise Timp1, BASP1 and SOD2. In some embodiments, the one or more FSGMs comprise Timp1, BASP1 and PSMB9. In some embodiments, the one or more FSGMs comprise Timp1, BASP1 and FTL. In some embodiments, the one or more FSGMs comprise Timp1, BASP1 and TXNIP. In some embodiments, the one or more FSGMs comprise LRRK2, CD44 and Xpo6. In some embodiments, the one or more FSGMs comprise LRRK2, CD44 and Lgals3. In some embodiments, the one or more FSGMs comprise LRRK2, CD44 and SOD2. In some embodiments, the one or more FSGMs comprise LRRK2, CD44 and PSMB9. In some embodiments, the one or more FSGMs comprise LRRK2, CD44 and FTL. In some embodiments, the one or more FSGMs comprise LRRK2, CD44 and TXNIP. In some embodiments, the one or more FSGMs comprise LRRK2, Xpo6 and Lgals3. In some embodiments, the one or more FSGMs comprise LRRK2, Xpo6 and SOD2. In some embodiments, the one or more FSGMs comprise LRRK2, Xpo6 and PSMB9. In some embodiments, the one or more FSGMs comprise LRRK2, Xpo6 and FTL. In some embodiments, the one or more FSGMs comprise LRRK2, Xpo6 and TXNIP. In some embodiments, the one or more FSGMs comprise LRRK2, Lgals3 and SOD2. In some embodiments, the one or more FSGMs comprise LRRK2, Lgals3 and PSMB9. In some embodiments, the one or more FSGMs comprise LRRK2, Lgals3 and FTL. In some embodiments, the one or more FSGMs comprise LRRK2, Lgals3 and TXNIP. In some embodiments, the one or more FSGMs comprise LRRK2, SOD2 and PSMB9. In some embodiments, the one or more FSGMs comprise LRRK2, SOD2 and FTL. In some embodiments, the one or more FSGMs comprise LRRK2, SOD2 and TXNIP. In some embodiments, the one or more FSGMs comprise BASP1, Xpo6 and Lgals3. In some embodiments, the one or more FSGMs comprise BASP1, Xpo6 and SOD2. In some embodiments, the one or more FSGMs comprise BASP1, Xpo6 and PSMB9. In some embodiments, the one or more FSGMs comprise BASP1, Xpo6 and FTL. In some embodiments, the one or more FSGMs comprise BASP1, Xpo6 and TXNIP. In some embodiments, the one or more FSGMs comprise BASP1, Lgals 3 and SOD2. In some embodiments, the one or more FSGMs comprise BASP1, Lgals3 and PSMB9. In some embodiments, the one or more FSGMs comprise BASP1, Lgals3 and FTL. In some embodiments, the one or more FSGMs comprise BASP1, Lgals3 and TXNIP. In some embodiments, the one or more FSGMs comprise BASP1, SOD2 and PSMB9. In some embodiments, the one or more FSGMs comprise BASP1, SOD2 and FTL. In some embodiments, the one or more FSGMs comprise BASP1, SOD2 and TXNIP. In some embodiments, the one or more FSGMs comprise Lgals3, SOD2 and PSMB9. In some embodiments, the one or more FSGMs comprise Lgals3, SOD2 and FTL. In some embodiments, the one or more FSGMs comprise Lgals3, SOD2 and TXNIP. In some embodiments, the one or more FSGMs comprise Timp1, CD44 and Xpo6. In some embodiments, the one or more FSGMs comprise Timp1, CD44 and Lgals3. In some embodiments, the one or more FSGMs comprise Timp1, CD44 and SOD2. In some embodiments, the one or more FSGMs comprise Timp1, CD44 and PSMB9. In some embodiments, the one or more FSGMs comprise Timp1, CD44 and FTL. In some embodiments, the one or more FSGMs comprise Timp1, CD44 and TXNIP. In some embodiments, the one or more FSGMs comprise Timp1, Xpo6 and Lgals3. In some embodiments, the one or more FSGMs comprise Timp1, Xpo6 and SOD2. In some embodiments, the one or more FSGMs comprise Timp1, Xpo6 and PSMB9. In some embodiments, the one or more FSGMs comprise Timp1, Xpo6 and FTL. In some embodiments, the one or more FSGMs comprise Timp1, Xpo6 and TXNIP. In some embodiments, the one or more FSGMs comprise Timp1, Lgals3 and SOD2. In some embodiments, the one or more FSGMs comprise Timp1, Lgals3 and PSMB9. In some embodiments, the one or more FSGMs comprise Timp1, Lgals3 and FTL. In some embodiments, the one or more FSGMs comprise Timp1, Lgals3 and TXNIP. In some embodiments, the one or more FSGMs comprise Timp1, SOD2 and PSMB9. In some embodiments, the one or more FSGMs comprise Timp1, SOD2 and FTL. In some embodiments, the one or more FSGMs comprise Timp1, SOD2 and TXNIP. In some embodiments, the one or more FSGMs comprise PSMB9, SOD2 and Lgals3. In some embodiments, the one or more FSGMs comprise PSMB9, SOD2 and Xpo6.

In some embodiments, the one or more FSGMs comprise Timp1, LRRK2, BASP1 and CD44. In some embodiments, the one or more FSGMs comprise Timp1, LRRK2, BASP1 and Xpo6. In some embodiments, the one or more FSGMs comprise Timp1, LRRK2, BASP1 and Lgals3. In some embodiments, the one or more FSGMs comprise Timp1, LRRK2, BASP1 and SOD2. In some embodiments, the one or more FSGMs comprise Timp1, LRRK2, BASP1 and PSMB9. In some embodiments, the one or more FSGMs comprise Timp1, LRRK2, BASP1 and FTL. In some embodiments, the one or more FSGMs comprise Timp1, LRRK2, BASP1 and TXNIP. In some embodiments, the one or more FSGMs comprise LRRK2, BASP1, CD44 and Xpo6. In some embodiments, the one or more FSGMs comprise LRRK2, BASP1, CD44 and Lgals3. In some embodiments, the one or more FSGMs comprise LRRK2, BASP1, CD44 and SOD2. In some embodiments, the one or more FSGMs comprise LRRK2, BASP1, CD44 and PSMB9. In some embodiments, the one or more FSGMs comprise LRRK2, BASP1, CD44 and FTL. In some embodiments, the one or more FSGMs comprise LRRK2, BASP1, CD44 and TXNIP. In some embodiments, the one or more FSGMs comprise BASP1, CD44, Xpo6 and Lgals3. In some embodiments, the one or more FSGMs comprise BASP1, CD44, Xpo6 and Lgals3. In some embodiments, the one or more FSGMs comprise BASP1, CD44, Xpo6 and SOD2. In some embodiments, the one or more FSGMs comprise BASP1, CD44, Xpo6 and PSMB9. In some embodiments, the one or more FSGMs comprise BASP1, CD44, Xpo6 and FTL. In some embodiments, the one or more FSGMs comprise BASP1, CD44, Xpo6 and TXNIP. In some embodiments, the one or more FSGMs comprise CD44, Xpo6, Lgals3 and SOD2. In some embodiments, the one or more FSGMs comprise CD44, Xpo6, Lgals3 and SOD2. In some embodiments, the one or more FSGMs comprise CD44, Xpo6, Lgals3 and PSMB9. In some embodiments, the one or more FSGMs comprise CD44, Xpo6, Lgals3 and FTL. In some embodiments, the one or more FSGMs comprise CD44, Xpo6, Lgals3 and TXNIP. In some embodiments, the one or more FSGMs comprise Xpo6, Lgals3, SOD2 and PSMB9. In some embodiments, the one or more FSGMs comprise Xpo6, Lgals3, SOD2 and FTL. In some embodiments, the one or more FSGMs comprise Xpo6, Lgals3, SOD2 and TXNIP. In some embodiments, the one or more FSGMs comprise PSMB9, SOD2, Lgals3 and Xpo6. In some embodiments, the one or more FSGMs comprise PSMB9, SOD2, Lgals3 and CD44. In some embodiments, the one or more FSGMs comprise PSMB9, SOD2, Lgals3 and BASP1. In some embodiments, the one or more FSGMs comprise PSMB9, SOD2, Lgals3 and LRRK2. In some embodiments, the one or more FSGMs comprise PSMB9, SOD2, Lgals3 and Timp1. In some embodiments, the one or more FSGMs comprise FTL, SOD2, Lgals3 and Xpo6. In some embodiments, the one or more FSGMs comprise FTL, SOD2, Lgals3 and CD44. In some embodiments, the one or more FSGMs comprise FTL, SOD2, Lgals3 and BASP1. In some embodiments, the one or more FSGMs comprise FTL, SOD2, Lgals3 and LRRK2. In some embodiments, the one or more FSGMs comprise FTL, SOD2, Lgals3 and Timp1. In some embodiments, the one or more FSGMs comprise TXNIP, SOD2, Lgals3 and Xpo6. In some embodiments, the one or more FSGMs comprise TXNIP, SOD2, Lgals3 and CD44. In some embodiments, the one or more FSGMs comprise TXNIP, SOD2, Lgals3 and BASP1. In some embodiments, the one or more FSGMs comprise TXNIP, SOD2, Lgals3 and LRRK2. In some embodiments, the one or more FSGMs comprise TXNIP, SOD2, Lgals3 and Timp1.

In some embodiments, the one or more FSGMs comprise Timp1, LRRK2, BASP1, CD44 and Xpo6. In some embodiments, the one or more FSGMs comprise Timp1, LRRK2, BASP1, CD44 and Lgals3. In some embodiments, the one or more FSGMs comprise Timp1, LRRK2, BASP1, CD44 and SOD2. In some embodiments, the one or more FSGMs comprise Timp1, LRRK2, BASP1, CD44 and PSMB9. In some embodiments, the one or more FSGMs comprise LRRK2, BASP1, CD44, Xpo6 and Lgals3. In some embodiments, the one or more FSGMs comprise LRRK2, BASP1, CD44, Xpo6 and SOD2. In some embodiments, the one or more FSGMs comprise LRRK2, BASP1, CD44, Xpo6 and PSMB9. In some embodiments, the one or more FSGMs comprise BASP1, CD44, Xpo6, Lgals3 and SOD2. In some embodiments, the one or more FSGMs comprise BASP1, CD44, Lgals3 and PSMB9. In some embodiments, the one or more FSGMs comprise CD44, Xpo6, Lgals3, SOD2 and PSMB9.

In some embodiments, the one or more FSGMs comprise Timp1, LRRK2, BASP1, CD44, Xpo6 and Lgals3. In some embodiments, the one or more FSGMs comprise Timp1, LRRK2, BASP1, CD44, Xpo6 and SOD2. In some embodiments, the one or more FSGMs comprise Timp1, LRRK2, BASP1, CD44, Xpo6 and PSMB9. In some embodiments, the one or more FSGMs comprise Timp1, LRRK2, BASP1, CD44, Xpo6 and FTL. In some embodiments, the one or more FSGMs comprise Timp1, LRRK2, BASP1, CD44, Xpo6 and TXNIP. In some embodiments, the one or more FSGMs comprise LRRK2, BASP1, CD44, Xpo6, Lgals3 and SOD2. In some embodiments, the one or more FSGMs comprise LRRK2, BASP1, CD44, Xpo6, Lgals3 and PSMB9. In some embodiments, the one or more FSGMs comprise LRRK2, BASP1, CD44, Xpo6, Lgals3 and FTL. In some embodiments, the one or more FSGMs comprise LRRK2, BASP1, CD44, Xpo6, Lgals3 and TXNIP. In some embodiments, the one or more FSGMs comprise Timp1, BASP1, CD44, Xpo6 Lgals3 and SOD2. In some embodiments, the one or more FSGMs comprise Timp1, BASP1, CD44, Xpo6 Lgals3 and PSMB9. In some embodiments, the one or more FSGMs comprise PSMB9, SOD2, Lgals3, Xpo6, CD44, and BASP1. In some embodiments, the one or more FSGMs comprise FTL, SOD2, Lgals3, Xpo6, CD44, and BASP1. In some embodiments, the one or more FSGMs comprise TXNIP, SOD2, Lgals3, Xpo6, CD44, and BASP1.

In some embodiments, the one or more FSGMs comprise Timp1, LRRK2, BASP1, CD44, Xpo6, Lgals3 and SOD2. In some embodiments, the one or more FSGMs comprise Timp1, BASP1, CD44, Xpo6, Lgals3, SOD2 and PSMB9. In some embodiments, the one or more FSGMs comprise Timp1, LRKK2, CD44, Xpo6, Lgals3, SOD2 and PSMB9. In some embodiments, the one or more FSGMs comprise Timp1, LRKK2, BASP1, Xpo6, Lgals3, SOD2 and PSMB9. In some embodiments, the one or more FSGMs comprise Timp1, LRKK2, BASP1, CD44, Lgals3, SOD2 and PSMB9. In some embodiments, the one or more FSGMs comprise Timp1, LRKK2, BASP1, CD44, Xpo6, SOD2 and PSMB9. In some embodiments, the one or more FSGMs comprise Timp1, LRKK2, BASP1, CD44, Xpo6, Lgals3, and PSMB9. In some embodiments, the one or more FSGMs comprise LRKK2, BASP1, CD44, Xpo6, Lgals3, SOD2 and PSMB9. In some embodiments, the one or more FSGMs comprise LRKK2, BASP1, CD44, Xpo6, Lgals3, SOD2 and FTL. In some embodiments, the one or more FSGMs comprise LRKK2, BASP1, CD44, Xpo6, Lgals3, SOD2 and TXNIP.

In some embodiments, the one or more FSGMs comprise Timp1, LRKK2, BASP1, CD44, Xpo6, Lgals3, SOD2 and PSMB9. In some embodiments, the one or more FSGMs comprise Timp1, LRKK2, BASP1, CD44, Xpo6, Lgals3, SOD2 and FTL. In some embodiments, the one or more FSGMs comprise Timp1, LRKK2, BASP1, CD44, Xpo6, Lgals3, SOD2 and TXNIP. In some embodiments, the one or more FSGMs comprise Timp1, LRKK2, BASP1, CD44, Xpo6, Lgals3, SOD2, PSMB9 and FTL. In some embodiments, the one or more FSGMs comprise Timp1, LRKK2, BASP1, CD44, Xpo6, Lgals3, SOD2, PSMB9 and TXNIP.

In some embodiments, any of the combinations of one or more of Timp1, LRKK2, BASP1, CD44, Xpo6, Lgals3, SOD2, PSMB9, FTL and TXNIP as described above may further be combined with one or more additional gene markers described in Table 6, i.e., EGR1, CTSS, BTG2 and/or PTGS2.

In some embodiments, any of the combinations of one or more of Timp1, LRKK2, BASP1, CD44, Xpo6, Lgals3, SOD2, PSMB9. FTL and TXNIP as described above may further be combined with EGR1. In some embodiments, any of the combinations of one or more of Timp1, LRKK2, BASP1, CD44, Xpo6, Lgals3, SOD2, PSMB9, FTL and TXNIP as described above may further be combined with CTSS. In some embodiments, any of the combinations of one or more of Timp1, LRKK2, BASP1, CD44, Xpo6, Lgals3, SOD2, PSMB9, FTL and TXNIP as described above may further be combined with BTG2. In some embodiments, any of the combinations of one or more of Timp1, LRKK2, BASP1, CD44, Xpo6, Lgals3, SOD2, PSMB9, FTL and TXNIP as described above may further be combined with PTGS2.

In some embodiments, any of the combinations of one or more of Timp1, LRKK2, BASP1, CD44, Xpo6, Lgals3, SOD2, PSMB9, FTL and TXNIP as described above may further be combined with EGR1 and CTSS. In some embodiments, any of the combinations of one or more of Timp1, LRKK2, BASP1, CD44, Xpo6, Lgals3, SOD2, PSMB9, FTL and TXNIP as described above may further be combined with EGR1 and BTG2. In some embodiments, any of the combinations of one or more of Timp1, LRKK2, BASP1, CD44, Xpo6, Lgals3, SOD2, PSMB9, FTL and TXNIP as described above may further be combined with EGR1 and PTGS2. In some embodiments, any of the combinations of one or more of Timp1, LRKK2, BASP1, CD44, Xpo6, Lgals3, SOD2, PSMB9, FTL and TXNIP as described above may further be combined with CTSS and BTG2. In some embodiments, any of the combinations of one or more of Timp1, LRKK2, BASP1, CD44, Xpo6, Lgals3, SOD2, PSMB9, FTL and TXNIP as described above may further be combined with CTSS and PTGS2. In some embodiments, any of the combinations of one or more of Timp1, LRKK2, BASP1, CD44, Xpo6, Lgals3, SOD2, PSMB9, FTL and TXNIP as described above may further be combined with BTG2 and PTGS2.

In some embodiments, any of the combinations of one or more of Timp1, LRKK2, BASP1, CD44, Xpo6, Lgals3, SOD2, PSMB9, FTL and TXNIP as described above may further be combined with EGR1, CTSS and BTG2. In some embodiments, any of the combinations of one or more of Timp1, LRKK2, BASP1, CD44, Xpo6, Lgals3, SOD2, PSMB9, FTL and TXNIP as described above may further be combined with EGR1, CTSS and PTGS2. In some embodiments, any of the combinations of one or more of Timp1, LRKK2, BASP1, CD44, Xpo6, Lgals3, SOD2, PSMB9, FTL and TXNIP as described above may further be combined with CTSS, BTG2 and PTGS2.

In some embodiments, any of the combinations of one or more of Timp1, LRKK2, BASP1, CD44, Xpo6, Lgals3, SOD2, PSMB9, FTL and TXNIP as described above may further be combined with EGR1, CTSS, BTG2 and PTGS2.

In one embodiment, methods provided by the present disclosure comprise determining an FXN expression profile for the one or more FSGMs described above. In another embodiment, methods provided by the present disclosure comprise determining an FXN expression profile for one or more FSGMs described above and additionally for one or more FSGMs defined in Tables 2, 3, 4 and/or 6. In one embodiment, the one or more FSGMs defined in Tables 2, 3, 4 and/or 6 comprise one or more of CTSS, BTG2, EGR1, and PTGS2. In one embodiment, the one or more FSGMs defined in Tables 2, 3, 4 and/or 6 comprise two or more of CTSS, BTG2, EGR1, and PTGS2. In one embodiment, the one or more FSGMs defined in Tables 2, 3, 4 and/or 6 comprise three or more of CTSS, BTG2, EGR1, and PTGS2. In one embodiment, the one or more FSGMs defined in Tables 2, 3, 4 and/or 6 comprise CTSS, BTG2, EGR1, and PTGS2.

In one aspect, the present disclosure provides a method of detecting one or more frataxin-sensitive genomic markers (FSGMs) in a sample from a frataxin (FXN) deficient subject, comprising contacting the sample, or a portion thereof, with one or more reagents specific for detecting the level of each of one or more FSGMs disclosed herein, thereby detecting the FSGMs in the sample. In one embodiment, the sample is a buccal sample. In one embodiment, the sample is a skin sample. In some embodiments, the subject has FRDA. In some embodiments, the FXN deficient subject is undergoing treatment or is scheduled to be treated with an FXN replacement therapy. In some embodiments, the one or more FSGMs comprise one or more FSGMs selected from Table 1 and/or Table 5. In some embodiments, the one or more FSGMs comprise one or more FSGMs selected from Table 1. In some embodiments, the one or more FSGMs comprise one or more FSGMs selected from Table 5. In some embodiments, the one or more FSGMs comprise one or more FSGMs selected from Table 1 and/or Table 5 in combination with one or more additional FSGMs listed in Tables 2, 3, 4 and/or 6. In some embodiments, the one or more FSGMs comprise one or more of Timp1, LRRK2, BASP1, CD44CD44, Xpo6, Lgals3, SOD2, and PSMB9, FTL and TXNIP. In some embodiments, the one or more FSGMs comprise one or more of Timp1, LRRK2, BASP1, CD44CD44, Xpo6, Lgals3, SOD2, and PSMB9, FTL and TXNIP in combination with one or more additional FSGMs listed in Tables 2, 3, 4 and/or 6, e.g., one or more of CTSS, BTG2, EGR1 and PTGS2. In some embodiments, the one or more FSGMs comprise or consist of Timp1, LRRK2, BASP1, CD44CD44, Xpo6, Lgals3, SOD2, PSMB9, FTL, TXNIP, CTSS, BTG2, EGR1 and PTGS2. In some embodiments, the one or more FSGMs comprise or consist of LRRK2 and Xpo6. In some embodiments, the one or more FSGMs comprise or consist of LRRK2, Xpo6, CTSS, BTG2, EGR1 and PTGS2.

In one embodiment, the one or more FSGMs comprise one or more of LRRK2, SOD2, CD44, and Lgals3. In another embodiment, the one or more FSGMs comprise two or more of LRRK2, SOD2, CD44, and Lgals3. In another embodiment, the one or more FSGMs comprise three or more of LRRK2, SOD2, CD44, and Lgals3. In another embodiment, the one or more FSGMs comprise LRRK2, SOD2, CD44, and Lgals3.

In one embodiment, the one or more FSGMs comprise one or more of LRRK2, CD44, and Lgals3. In another embodiment, the one or more FSGMs comprise two or more of LRRK2, CD44, and Lgals3. In another embodiment, the one or more FSGMs comprise LRRK2, CD44, and Lgals3.

In one embodiment, the present disclosure provides a method of detecting one or more frataxin-sensitive genomic markers (FSGMs) in a buccal sample from a frataxin (FXN) deficient subject (e.g., a subject having FRDA), comprising contacting the sample, or a portion thereof, with one or more reagents specific for detecting the level of each of one or more FSGMs disclosed herein, wherein the FSGMs comprise one or more FSGMs selected from Table 5, thereby detecting the FSGMs in the sample.

In one embodiment, the present disclosure provides a method of detecting one or more frataxin-sensitive genomic markers (FSGMs) in a buccal sample from a frataxin (FXN) deficient subject (e.g., a subject having FRDA) undergoing FXN replacement therapy, comprising contacting the sample, or a portion thereof, with one or more reagents specific for detecting the level of each of one or more FSGMs disclosed herein, wherein the FSGMs comprise one or more FSGMs selected from Table 5, thereby detecting the FSGMs in the sample.

In one embodiment, methods provided by the present disclosure comprise determining an FXN expression profile for the one or more FSGMs described above. In another embodiment, methods provided by the present disclosure comprise determining an FXN expression profile for one or more FSGMs described above and additionally for one or more FSGMs defined in Tables 2, 3, 4 and/or 6. In one embodiment, the one or more FSGMs defined in Tables 2, 3, 4 and/or 6 comprise one or more of CTSS, BTG2, EGR1, and PTGS2. In one embodiment, the one or more FSGMs defined in Tables 2, 3, 4 and/or 6 comprise two or more of CTSS, BTG2, EGR1, and PTGS2. In one embodiment, the one or more FSGMs defined in Tables 2, 3, 4 and/or 6 comprise three or more of CTSS, BTG2, EGR1, and PTGS2. In one embodiment, the one or more FSGMs defined in Tables 2, 3, 4 and/or 6 comprise CTSS, BTG2, EGR1, and PTGS2.

By way of example, an FXN expression profile may be determined through the measurement of expression levels of at least one or any combination of more than one FSGM. As used herein, an FSGM includes any one or more of the FSGMs listed in Table 1 and/or Table 5, alone or in combination with one or more additional FSGMs listed in Tables 2, 3, 4 and/or 6.

Hereinafter an expression profile may also be referred to as a signature.

In one embodiment of the disclosure, a baseline FXN(−) expression profile comprises altered expression of at least one or any combination of more than one FSGM, e.g., any one or more of the FSGMs listed in Table 1 and/or Table 5, alone or in combination with one or more additional FSGMs listed in Tables 2, 3, 4 and/or 6.

In another embodiment of the disclosure, a baseline FXN(−) expression profile may comprise the downregulated expression levels of at least one of Timp1, LRRK2, BASP1, CD44, Xpo6, SOD2, PSMB9, FTL and TXNIP, or any combination thereof. A measure of effectiveness of FXN replacement therapy may be indicated by a pattern of upregulation of any one or more of these FSGMs.

In another embodiment of the disclosure, a baseline FXN(−) expression profile may comprise the upregulated expression level of Lgals3. A measure of effectiveness of FXN replacement therapy may be indicated by a pattern of downregulation of Lgals3.

In one embodiment, an FXN replacement expression profile comprises the reversed expression of a baseline FXN(−) expression profile.

In another embodiment, an FXN replacement expression profile for use as an indicator of FXN replacement treatment effectiveness may comprise one or any combination of two or more of the FSGMs presented in Table 1 and/or Table 5 alone, including, for example, one or more of Timp1, LRRK2, BASP1, CD44, Xpo6, SOD2, PSMB9, FTL and TXNIP, CTSS, BTG2, EGR1 and PTGS2, detected in a sample from a subject treated with FXN replacement therapy.

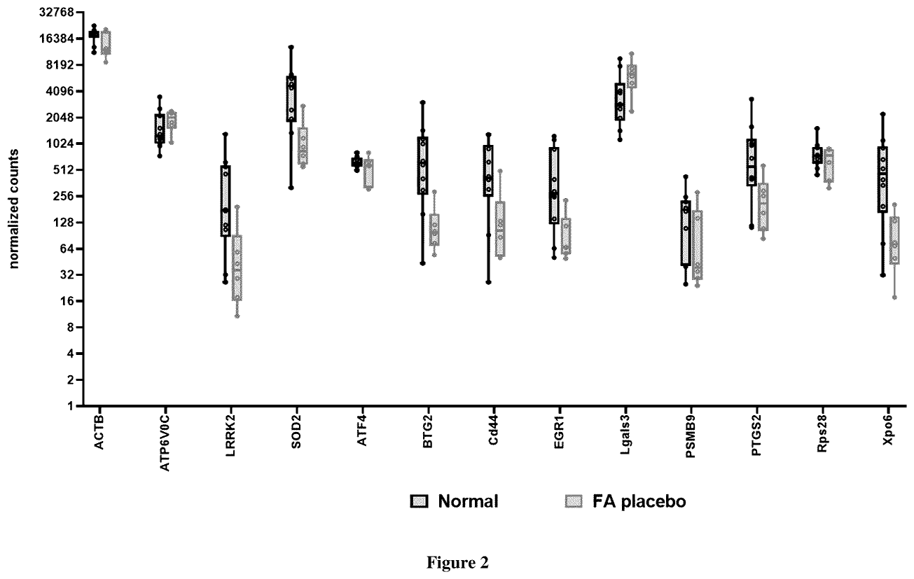

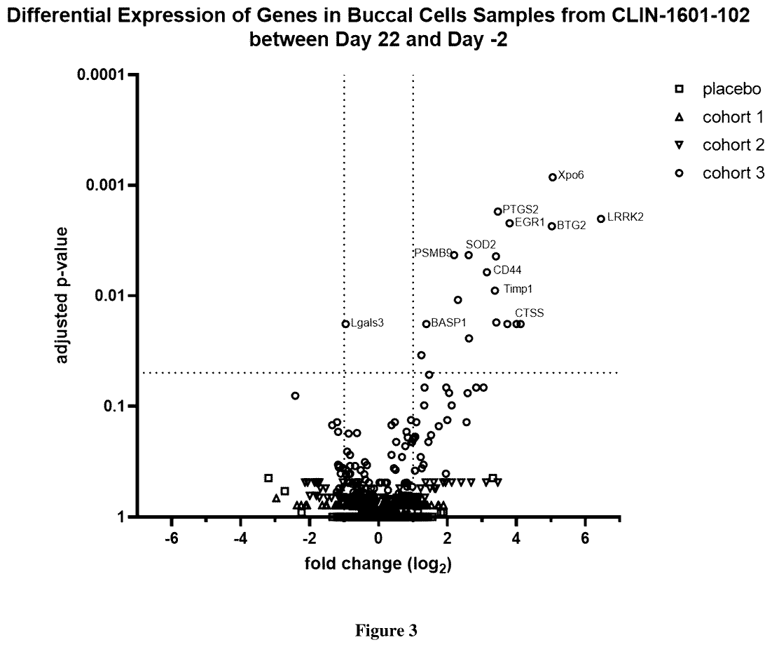

In another embodiment, an FXN replacement expression profile may comprise an expression pattern exemplified in Table 2 and FIGS. 2, 3 and/or 4 by fold regulation of FXN replacement expression profile in subjects treated with FXN replacement therapy.

In some embodiments, an FXN replacement expression profile is characterized by the contrary regulation of FSGMs, which is defined by any FSGMs that were downregulated in FXN depletion conditions, e.g., in a subject with FRDA, that become upregulated following FXN replacement therapy; and the reverse is also valid, such that any FSGMs that were upregulated in FXN depletion conditions, e.g., in a subject with FRDA, become downregulated following FXN replacement therapy. Accordingly, detection of altered expression of one or more FSGMs in a sample following FXN replacement therapy allows for monitoring of efficacy of the FXN replacement therapy in a subject. For example, in one embodiment, a lack of altered expression of one or more FSGMs in a sample following FXN replacement therapy indicates that the FXN replacement therapy may not have been successful and/or that increased FXN replacement therapy may be needed. Likewise, in another embodiment, altered expression of one or more FSGMs in a sample following FXN replacement therapy indicates that FXN replacement therapy was successful.

Altered or modulated expression may comprise increased expression, also referred to as overexpression or upregulation, or decreased or inhibited expression, also referred to as downregulation.

As referred to herein, feature vectors are a set of values that characterize an expression profile. Feature vectors may comprise a set of n FSGMs, n being the number of different genes whose expression levels were measured in a sample. By way of example, n may be all the FSGMs provided in Table 1 and/or Table 5, alone or in combination with one or more additional FSGMs listed in Tables 2, 3, 4 and/or 6. Alternatively, n may be at least one, two, or three, or four, or five, or six, or any number of FSGMs presented in Table 1 and/or Table 5, alone or in combination with one or more additional FSGMs listed in Tables 2, 3, 4 and/or 6, in any combination.

In one embodiment, a set of FSGMs may comprise at least one or any combination of more than one FSGM, e.g., any one or more of the FSGMs listed in Table 1 and/or Table 5, alone or in combination with one or more additional FSGMs listed in Tables 2, 3, 4 and/or 6. For example, one or more of FSGMs comprise one or more of Timp1, LRRK2, BASP1, CD44, Xpo6, SOD2, PSMB9, FTL, TXNIP, BTG2, EGR1 and PTGS2, alone or in combination with one or more additional FSGMs listed in Tables 2, 3, 4 and/or 6, e.g., one or more of CTSS, BTG2, EGR1 and PTGS2, in any combination.

In one embodiment, the one or more FSGMs comprise one or more of Timp1, LRRK2, BASP1, CD44, Xpo6, Lgals3, SOD2, PSMB9, FTL and TXNIP. In another embodiment, the one or more FSGMs comprise two or more of Timp1, LRRK2, BASP1, CD44, Xpo6, Lgals3, SOD2, PSMB9, FTL and TXNIP. In another embodiment, the one or more FSGMs comprise three or more of Timp1, LRRK2, BASP1, CD44, Xpo6, Lgals3, SOD2, PSMB9, FTL and TXNIP. In another embodiment, the one or more FSGMs comprise four or more of Timp1, LRRK2, BASP1, CD44, Xpo6, Lgals3, SOD2, PSMB9, FTL and TXNIP. In another embodiment, the one or more FSGMs comprise five or more of Timp1, LRRK2, BASP1, CD44, Xpo6, Lgals3, SOD2, PSMB9, FTL and TXNIP. In another embodiment, the one or more FSGMs comprise six or more of Timp1, LRRK2, BASP1, CD44, Xpo6, Lgals3, SOD2, PSMB9, FTL and TXNIP. In another embodiment, the one or more FSGMs comprise seven or more of Timp1, LRRK2, BASP1, CD44, Xpo6, Lgals3, SOD2, PSMB9, FTL and TXNIP. In another embodiment, the one or more FSGMs comprise eight or more of Timp1, LRRK2, BASP1, CD44, Xpo6, Lgals3, SOD2, PSMB9, FTL and TXNIP. In another embodiment, the one or more FSGMs comprise nine or more of Timp1, LRRK2, BASP1, CD44, Xpo6, Lgals3, SOD2, PSMB9, FTL and TXNIP. In another embodiment, the one or more FSGMs comprise Timp1, LRRK2, BASP1, CD44, Xpo6, Lgals3, SOD2, PSMB9, FTL and TXNIP.

In one embodiment, the one or more FSGMs comprise one or more of Timp1, LRRK2, BASP1, CD44, Xpo6, Lgals3, PSMB9, FTL and TXNIP. In another embodiment, the one or more FSGMs comprise two or more of Timp1, LRRK2, BASP1, CD44, Xpo6, Lgals3, PSMB9, FTL and TXNIP. In another embodiment, the one or more FSGMs comprise three or more of Timp1, LRRK2, BASP1, CD44, Xpo6, Lgals3, PSMB9, FTL and TXNIP. In another embodiment, the one or more FSGMs comprise four or more of Timp1, LRRK2, BASP1, CD44, Xpo6, Lgals3, PSMB9, FTL and TXNIP. In another embodiment, the one or more FSGMs comprise five or more of Timp1, LRRK2, BASP1, CD44, Xpo6, Lgals3, PSMB9, FTL and TXNIP. In another embodiment, the one or more FSGMs comprise six or more of Timp1, LRRK2, BASP1, CD44, Xpo6, Lgals3, PSMB9, FTL and TXNIP. In another embodiment, the one or more FSGMs comprise seven or more of Timp1, LRRK2, BASP1, CD44, Xpo6, Lgals3, PSMB9, FTL and TXNIP. In another embodiment, the one or more FSGMs comprise Timp1, LRRK2, BASP1, CD44, Xpo6, Lgals3, PSMB9, FTL and TXNIP.

In one embodiment, methods provided by the present disclosure comprise determining an FXN expression profile for the one or more FSGMs described above. In another embodiment, methods provided by the present disclosure comprise determining an FXN expression profile for one or more FSGMs described above and additionally for one or more FSGMs defined in Tables 2, 3, 4 and/or 6. In one embodiment, the one or more FSGMs defined in Tables 2, 3, 4 and/or 6 comprise one or more of CTSS, BTG2, EGR1, and PTGS2. In one embodiment, the one or more FSGMs defined in Tables 2, 3, 4 and/or 6 comprise two or more of CTSS, BTG2, EGR1, and PTGS2. In one embodiment, the one or more FSGMs defined in Tables 2, 3, 4 and/or 6 comprise three or more of CTSS, BTG2, EGR1, and PTGS2. In one embodiment, the one or more FSGMs defined in Tables 2, 3, 4 and/or 6 comprise CTSS, BTG2, EGR1, and PTGS2.

In one embodiment, the one or more FSGMs comprise one or more of LRRK2, SOD2, CD44, and Lgals3. In another embodiment, the one or more FSGMs comprise two or more of LRRK2, SOD2, CD44, and Lgals3. In another embodiment, the one or more FSGMs comprise three or more of LRRK2, SOD2, CD44, and Lgals3. In another embodiment, the one or more FSGMs comprise LRRK2, SOD2, CD44, and Lgals3.

In one embodiment, the one or more FSGMs comprise one or more of LRRK2, CD44, and Lgals3. In another embodiment, the one or more FSGMs comprise two or more of LRRK2, CD44, and Lgals3. In another embodiment, the one or more FSGMs comprise LRRK2, CD44, and Lgals3.

By way of example, a normal FXN expression profile, obtained from samples of healthy subjects, may be comprised of expression levels of a set of FSGMs, and may be represented by and referred to as a normal FXN feature vector. As described in the following examples, FSGMs when measured in FXN deficient samples, e.g., samples from subject with FRDA, may present expression levels that are different from the levels of expression of FSGMs in healthy subjects, and thus may be represented by and referred to as a deficient FXN feature vector. In one embodiment, the difference between a deficient FXN feature vector and a normal FXN feature vector may be detected and quantified by the distance between the two feature vectors. In an alternative scenario, expression levels of FSGMs from a sample from an FXN deficient subject, e.g., subject with FRDA, following FXN replacement treatment may present yet different expression levels, and may be represented by and referred to as an FXN replacement feature vector. As for the previous two feature vectors, the difference between an FXN replacement feature vector and either a normal FXN feature vector or a deficient FXN feature vector may be detected and quantified by the distance between the replacement FXN feature vector and the normal FXN feature vector or the deficient FXN feature vector.

As such, having a sample from an FXN deficient subject obtained prior to treatment and a sample obtained post-FXN replacement treatment, a first FXN feature vector may be determined for the FXN replacement expression profile and a second FXN feature vector may be determined for the baseline FXN(−) expression profile; wherein determining a distance, or scalar product, between the first and the second feature vectors may be used for determining effectiveness of the FXN replacement therapy. In an embodiment of the disclosure, a third feature vector may be determined for the normal FXN expression profile, the normal expression profile being established for the FSGMs in a sample from a healthy subject. In an embodiment, the distance between the second (baseline FXN(−) expression profile) and third (normal FXN expression profile) FXN feature vectors may be determined. In another embodiment, the distance between the first (FXN replacement expression profile) and third (normal FXN expression profile) FXN feature vectors may be determined, and may be used for determining effectiveness of the FXN replacement therapy. In an embodiment, the distance between the first and third feature vectors may be normalized to the distance between the second and third feature vectors, and the resulting normalized distance may be used to determine effectiveness of the FXN replacement therapy. In an embodiment, the resulting normalized distance may be a value ranging from 0 (zero) to 1 (one), wherein the smaller the value (closest to zero) the more effective the therapy.

The markers of the disclosure, e.g., one or more FSGMs selected from Table 1 and/or Table 5, alone or in combination with one or more FSGMs selected from Tables 2, 3, 4 and/or 6, are correlated with FXN levels in a subject. Accordingly, in one aspect, the present invention provides methods for using, measuring, detecting, quantifying, and the like of one or more of the FSGMs in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more FSGMs listed in Tables 2, 3, 4 and/or 6, such as one or more of CTSS, BTG2, EGR1 and PTGS2, for determining and/or monitoring the FXN status in a subject or for determining, evaluating, and/or monitoring FXN replacement therapy in a subject.

In another aspect, the present invention relates to using, measuring, detecting, quantifying, and the like of one or more of the FSGMs in Table 1 and/or Table 5, alone or in combination with one or more additional FSGMs listed in Tables 2, 3, 4 and/or 6 (e.g., one or more of Timp1, LRRK2, BASP1, CD44, Xpo6, Lgals3, SOD2, PSMB9, FTL and TXNIP, alone or in combination with one or more FSGMs listed in Tables 2, 3, 4 and/or 6, such as one or more of CTSS, BTG2, EGR1 and PTGS2) as a surrogate for measuring FXN expression levels.

In addition, in another embodiment, the FSGMs may be used in combination with one or more additional markers for a mitochondrial disease, e.g., FRDA. Other markers that may be used in combination with the one or more FSGMs listed in Table 1 and/or Table 5, alone or in combination with one or more additional FSGMs listed in Tables 2, 3, 4 and/or 6, include any measurable characteristic described herein that reflects in a quantitative or qualitative manner the physiological state of an organism, e.g., whether the organism has a mitochondrial disease, e.g., FRDA. The physiological state of an organism is inclusive of any disease or non-disease state, e.g., a subject having a mitochondrial disease, e.g., FRDA, or a subject who is otherwise healthy. The FSGMs of the invention that may be used in combination with one or more additional FSGMs in Tables 2, 3, 4 and/or 6, include characteristics that can be objectively measured and evaluated as indicators of normal processes, pathogenic processes, or pharmacologic responses to a therapeutic intervention. Such combination markers can be clinical parameters (e.g., age, performance status), laboratory measures (e.g., molecular markers), or genetic or other molecular determinants. In other embodiments, the present invention also involves the analysis and consideration of any clinical and/or subject-related health data, for example, data obtained from an Electronic Medical Record (e.g., collection of electronic health information about individual subjects or populations relating to various types of data, such as, demographics, medical history, medication and allergies, immunization status, laboratory test results, radiology images, vital signs, personal statistics like age and weight, and billing information).

The present invention also contemplates the use of particular combinations of the FSGMs listed in Table 1 and/or Table 5, alone or in combination with one or more additional FSGMs listed in Tables 2, 3, 4 and/or 6, e.g., combinations of FSGMs comprise one or more of Timp1, LRRK2, BASP1, CD44, Xpo6, Lgals3, SOD2, PSMB9, FTL and TXNIP, alone or in combination with one or more FSGMs listed in Tables 2, 3, 4 and/or 6, e.g., one or more of CTSS, BTG2, EGR1 and PTGS2, in any combination. In one embodiment, the invention contemplates FSGM sets with at least two (2) members, which may include any two of the FSGMs listed in Table 1 and/or Table 5, alone or in combination with one or more additional FSGMs listed in Tables 2, 3, 4 and/or 6. In another embodiment, the invention contemplates FSGM sets with at least three (3) members, which may include any three of the FSGMs listed in Table 1 and/or Table 5, alone or in combination with one or more additional FSGMs listed in Tables 2, 3, 4 and/or 6. In another embodiment, the invention contemplates FSGM sets with at least four (4) members, which may include any four of the FSGMs listed in Table 1 and/or Table 5, alone or in combination with one or more additional FSGMs listed in Tables 2, 3, 4 and/or 6. In another embodiment, the invention contemplates FSGM sets with at least five (5) members, which may include any five of the FSGMs listed in Table 1 and/or Table 5, alone or in combination with one or more additional FSGMs listed in Tables 2, 3, 4 and/or 6. In another embodiment, the invention contemplates FSGM sets with at least six (6) members, which may include any six of the FSGMs listed in Table 1 and/or Table 5, alone or in combination with one or more additional FSGMs listed in Tables 2, 3, 4 and/or 6. In another embodiment, the invention contemplates FSGM sets with at least seven (7) members, which may include any seven of the FSGMs listed in Table 1 and/or Table 5, alone or in combination with one or more additional FSGMs listed in Tables 2, 3, 4 and/or 6. In another embodiment, the invention contemplates FSGM sets with at least eight (8) members, which may include any eight of the FSGMs listed in Table 1 and/or Table 5, alone or in combination with one or more additional FSGMs listed in Tables 2, 3, 4 and/or 6. In another embodiment, the invention contemplates FSGM sets with at least nine (9) members, which may include any nine of the FSGMs listed in Table 1 and/or Table 5, alone or in combination with one or more additional FSGMs listed in Tables 2, 3, 4 and/or 6. In another embodiment, the invention contemplates FSGM sets with at least ten (10) members, which may include any ten of the FSGMs listed in Table 1 and/or Table 5, alone or in combination with one or more additional FSGMs listed in Tables 2, 3, 4 and/or 6. In another embodiment, the invention contemplates FSGM sets with at least eleven (11) members, which may include any eleven of the FSGMs listed in Table 1 and/or Table 5, alone or in combination with one or more additional FSGMs listed in Tables 2, 3, 4 and/or 6. In another embodiment, the invention contemplates FSGM sets with at least twelve (12) members, which may include any twelve of the FSGMs listed in Table 1 and/or Table 5, alone or in combination with one or more additional FSGMs listed in Tables 2, 3, 4 and/or 6. In other embodiments, the invention contemplates an FSGM set comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, or 138 of the FSGMs listed in Table 1 and/or Table 5, alone or in combination with one or more additional FSGMs listed in Tables 2, 3, 4 and/or 6. In one embodiment, the invention contemplates FSGM sets comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, or 138 of the FSGMs listed in Table 1 and/or Table 5, alone or in combination with one or more additional FSGMs listed in Tables 2, 3, 4 and/or 6, wherein one or more of the FSGMs in the set is a FSGM defined in Table 1 and/or Table 5, e.g., Timp1, LRRK2, BASP1, CD44, Xpo6, Lgals3, SOD2, PSMB9, FTL and TXNIP. In some embodiments, the one or more FSGMs comprise Xpo6, SOD2, CTSS, EGR1, BTG2, PTSG2 and LRKK2. In some embodiments, the one or more FSGMs comprise Xpo6, SOD2, CTSS, EGR1, BTG2, PTSG2 and LRKK2.

In another embodiment, the invention contemplates FSGM sets comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, or 102 of the FSGMs listed in Table 1 and/or Table 5, alone or in combination with one or more additional FSGMs listed in Tables 2, 3, 4 and/or 6, wherein one of the FSGMs in the set is Timp1, LRRK2, BASP1, CD44, Xpo6, Lgals3, SOD2, PSMB9, FTL and TXNIP, together with CTSS, BTG2, EGR1 and PTGS2. In some embodiments, the one or more FSGMs comprise Xpo6, SOD2, CTSS, EGR1, BTG2, PTSG2 and LRKK2.

In certain embodiments, the level of the FSGM is increased following treatment of a subject with FXN replacement, e.g., a subject deficient in FXN. In some embodiments, the FSGM is selected from the group consisting of Timp1, Timp1, LRRK2, BASP1, CD44, Xpo6, SOD2, PSMB9, FTL and TXNIP.

In other embodiments, the level of a FSGM is decreased following treatment of a subject with FXN replacement, e.g., a subject deficient in FXN. In some embodiments, the FSGM is Lgals3.

In another aspect, the present invention provides for the identification of a “diagnostic signature” or “diagnostic expression profile” based on the levels of the FSGMs of the invention in a biological sample, that correlates with FXN in the sample. The “levels of the FSGMs” can refer to the protein level of an FSGM in a biological sample. The “levels of the FSGMs” can also refer to the expression level of the genes corresponding to the proteins, e.g., by measuring the expression levels of the corresponding FSGM mRNAs. The collection or totality of levels of FSGMs provide a diagnostic signature that correlates with the level of FXN.

In certain embodiments, the diagnostic signature is obtained by (1) detecting the level of at least 1, 2, 3, 4, 5, 6, 7, 8, 9, 10 or more FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or together with one or more of CTSS, BTG2, EGR1 and PTGS2, in a biological sample from a subject receiving FXN replacement therapy; (2) comparing the levels of the at least 1, 2, 3, 4, 5, 6, 7, 8, 9, 10 or more FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or together with one or more of CTSS, BTG2, EGR1 and PTGS2, to the levels of the same FSGMs from a control sample, such as a baseline FXN(−) expression profile; and (3) determining if the at least 1, 2, 3, 4, 5, 6, 7, 8, 9, 10 or more FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or together with one or more of CTSS, BTG2, EGR1 and PTGS2, detected in the biological sample are above or below the levels of the FSGMs in the control (e.g., baseline FXN(−) expression profile). If the at least 1, 2, 3, 4, 5, 6, 7, 8, 9, 10 or more FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or together with one or more of CTSS, BTG2, EGR1 and PTGS2, are above or below the control (e.g., baseline FXN(−) expression profile), then the diagnostic signature is indicative of the effectiveness of FXN replacement therapy. In some embodiments, the one or more FSGMs comprise Xpo6, SOD2, CTSS, EGR1, BTG2, PTSG2 and LRKK2.

In accordance with various embodiments, algorithms may be employed to predict whether or not a biological sample from a subject comprises FXN, or to evaluate or monitor whether the subject has effectively received FXN replacement therapy. The skilled artisan will appreciate that an algorithm can be any computation, formula, statistical survey, nomogram, look-up Tables, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output.” Any suitable algorithm—whether computer-based or manual-based (e.g., look-up Tables)—is contemplated herein.

In certain embodiments, the FSGMs of the invention, e.g., one or more of Timp1, LRRK2, BASP1, CD44, Xpo6, Lgals3, SOD2, PSMB9, FTL and TXNIP, alone or together with one or more of CTSS, BTG2, EGR1 and PTGS2, can include variant sequences. More particularly, certain binding agents/reagents used for detecting certain of the FSGMs of the invention can bind and/or identify variants of these certain FSGMs of the invention. As used herein, the term “variant” encompasses nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

Variant sequences generally differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical amino acids or nucleotides in a specified region may be determined against another polypeptide or polynucleotide sequence, using computer algorithms that are publicly available. The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

Two exemplary algorithms for aligning and identifying the identity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0× that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

In an alternative embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The invention provides for the use of various combinations and sub-combinations of FSGMs. For example, one or more of FSGMs listed in Table 1 and/or Table 5, e.g., one or more of Timp1, LRRK2, BASP1, CD44, Xpo6, Lgals3, SOD2, PSMB9, FTL and TXNIP, may be used alone or in combination with one or more FSGMs listed in Tables 2, 3, 4 and/or 6, e.g., one or more of CTSS, BTG2, EGR1 and PTGS2, in any combination. It is understood that any single FSGM or combination of the FSGMs provided herein can be used in the invention unless clearly indicated otherwise.

### D. Biological Samples

The present invention may be practiced with any suitable biological sample that potentially contains, expresses, includes, a detectable FSGM. For example, the biological sample may be obtained from a solid tissue sample, such as a skin biopsy sample, muscle biopsy sample, preferably a buccal sample, or a body fluid sample such as blood (including any blood product, such as whole blood, plasma, serum, or specific types of cells of the blood), urine, saliva, or seminal fluid. Alternatively, a sample can comprise exosomes which may be harvested in order to be tested for FSGM transcripts.

In some embodiments, a sample which may be used for measuring an FXN expression profile in the context of the present disclosure may be selected from the group consisting of a buccal sample, a skin sample, a hair follicle and a muscle biopsy sample. In one embodiment, the sample is a buccal sample. In another embodiment, the sample is a skin sample. In some embodiments, the sample is a serum sample.

In some embodiments, a sample which may be used for measuring an FXN expression profile may be obtained from an FXN deficient subject prior to administration of FXN replacement therapy, during administration of FXN replacement therapy or after administration of FXN replacement therapy. In some embodiments, a sample may be obtained from an FXN deficient subject at least 15 days following the last administration of the FXN replacement therapy. In some embodiments, a sample may be obtained from an FXN deficient subject 15 to 45 days following the last administration of the FXN replacement therapy, e.g., 15 to 20 days, 20 to 35 days or 25 to 45 days after the last administration of the FXN replacement therapy. In some embodiments, a sample may be obtained from an FXN deficient subject 20 to 25 days, e.g., 20, 21, 22, 23 or 25 days, after the last administration of the FXN replacement therapy. In some embodiments, a sample may be obtained from an FXN deficient subject at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days after the last administration of the FXN replacement therapy.

The inventive methods may be performed at the single cell level. However, the inventive methods may also be performed using a sample comprising many cells, where the assay is “averaging” expression over the entire collection of cells and tissue present in the sample. Preferably, there is enough of the tissue sample to accurately and reliably determine the expression levels of interest.

Any commercial device or system for isolating and/or obtaining tissue and/or blood or other biological products, and/or for processing said materials prior to conducting a detection reaction is contemplated.

In certain embodiments, the present invention relates to detecting FSGM nucleic acid molecules (e.g., mRNA encoding one or more FSGMs listed in Table 1 and/or Table 5, alone or in combination with one or more additional FSGMs listed in Tables 2, 3, 4 and/or 6, for example, one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2). In such embodiments, RNA can be extracted from a biological sample before analysis. Methods of RNA extraction are well known in the art (see, for example, J. Sambrook et al., “Molecular Cloning: A Laboratory Manual”, 1989, 2nd Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors. Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5.sup.th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

In certain embodiments, the RNA isolated from the tissue sample (for example, after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). Preferably, the detectable agent is selected such that it generates a signal which can be measured and whose intensity is related to the amount of labeled nucleic acids present in the sample being analyzed. In array-based analysis methods, the detectable agent is also preferably selected such that it generates a localized signal, thereby allowing spatial resolution of the signal from each spot on the array.

Methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

However, in some embodiments, the expression levels are determined by detecting the expression of a gene product (e.g., a protein, such as a secreted protein) thereby eliminating the need to obtain a genetic sample (e.g., RNA) from the sample.

### E. Detection and/or Measurement of FSGMs

Various methodologies may be utilized for measuring the distance between feature vectors. Once the data is normalized, the distance may be achieved for example by calculating the mean squared error, which may be extracted from the difference in the expression pattern of each gene measured in two different profiles, such as baseline FXN(−) and FXN replacement for example. Alternatively, the distance may be achieved by calculating a correlation coefficient or applying a t-test.

As described in detail herein, many methodologies have been described for the determination of RNA expression profiles, including sequencing, hybridization or amplification of the sample RNA. In a particular embodiment of the disclosure, said determining the expression profile of a sample of a subject comprises obtaining or provided a biological sample from a subject, extracting RNA from the sample, generating the corresponding cDNA, and detecting expression profile through any one of sequencing, hybridization or amplification. In some embodiments, the expression profile of a sample may be determined using qPCR. In some embodiments, the expression profile of a sample may be determined using Nanostring.

Detecting the FXN-sensitive expression profiles by sequencing may use, for example, next generation sequencing (NGS), RNASeq, and any sequencing techniques known to the man skilled in the art.

Detecting expression profile by hybridization comprises contacting a subject sample, or a portion thereof, with a probe or a set of probes that specifically hybridize with FSGMs (or their transcripts) disclosed in Table 1 and/or Table 5, alone or in combination with one or more additional FSGMs listed in Tables 2, 3, 4 and/or 6. In one embodiment of the disclosure, any one or more FSGMs of the disclosure or any combination thereof, may be contacted with a subject sample. By way of example, specific probes for at least one of the transcripts of genes encoding one or more of FSGMs listed in Table 1 and/or Table 5, e.g., one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2, or any combination thereof, may be contacted with a subject sample. Thus, determining the expression profile of a sample of a subject treated with FXN replacement therapy by hybridization may comprise contacting the sample, or a portion thereof, with probes that hybridize with at least 75%, 80%, 85%, 90%, 95%, 97%, 98% or 99% of its target nucleic acid, the target nucleic acid being the transcript, or the corresponding cDNA for any one of the FSGMs listed in Table 1 and/or Table 5, e.g., one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2.

Detecting expression profile by amplification involves, by way of example, polymerase chain reaction techniques, such as real-time polymerase chain reaction (RT-PCR), which comprises contacting the sample with forward and reverse primers for each of the transcripts of interest as exemplified herein below in the examples and generating RT-PCR products. Optionally RT-PCR products are detected with specific or general probes, or a combination thereof, which facilitate their quantification. Thus, an FXN-induced signature may be determined by detecting FSGMs transcripts in a sample. In an embodiment of the disclosure, forward and reverse primers are used for detecting FSGMs transcripts.

In an alternative embodiment the expression profile may be detected through FSGMs protein products and analysis of protein profile, which may be performed through protein detection methodology, using techniques involving specific antibodies, or protein quantification/characterization techniques, such as high-performance liquid chromatography (HPLC), mass spectrometry-based techniques, gel-based techniques for example differential in-gel electrophoresis, and the like.

In another aspect, the present disclosure provides a composition for detection of an FXN expression profile, the composition comprising at least one or a plurality of nucleotide sequences for detection of FSGMs. In one embodiment of the disclosure, the composition may be for detection of any one of an FXN replacement expression profile, a baseline FXN(−) expression profile and/or a normal FXN expression profile. The composition may comprise at least one nucleotide sequence for the detection of transcripts of the genes defined in Table 1 and/or Table 5, alone or in combination with one or more additional FSGMs listed in Tables 2, 3, 4 and/or 6. The composition may comprise nucleotide sequences for the detection of two, three, four, five, six, seven, eight, nine, ten, fifteen, twenty, thirty, forty, and/or up to all FSGMs presented in Table 1 and/or Table 5, alone or in combination with one or more additional FSGMs listed in Tables 2, 3, 4 and/or 6. By way of example, a composition for detection of an FXN signature comprises nucleotides for detection of one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2, or any combination thereof. A nucleotide sequence may be DNA or an analog thereof, or RNA or an analog thereof. The nucleotide sequence may be complementary to as least a portion of an FSGM. Binding of the nucleotide sequence for detection of FSGMs will depend on the level of stringency of the reaction. The nucleotide sequence may be an oligonucleotide, which may function as a probe or a primer, and as such may comprise modifications compatible with their function. For example, short oligonucleotides used as probes may carry labels, for example fluorescent labels, that enable their detection and quantification.

The present invention contemplates any suitable means, techniques, and/or procedures for detecting and/or measuring the FSGMs of the invention. These methods are described in detail below.

1. Detection of Protein FSGMs

The present invention contemplates any suitable method for detecting polypeptide FSGMs listed in Table 1 and/or Table 5, alone or in combination with one or more additional FSGMs listed in Tables 2, 3, 4 and/or 6. In certain embodiments, the detection method is an immunodetection method involving an antibody that specifically binds to one or more of the FSGMs listed in Table listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2. The steps of various useful immunodetection methods have been described in the scientific literature, such as, e.g., Nakamura et al. (1987), which is incorporated herein by reference.

In general, the immunobinding methods include obtaining a sample suspected of containing an FSGM protein, peptide, e.g., an FSGM secreted protein or peptide, or antibody, and contacting the sample, or a portion thereof, with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing an FSGM protein, peptide or a corresponding antibody, and contact the sample with an antibody or encoded protein or peptide, as the case may be, and then detect or quantify the amount of immune complexes formed under the specific conditions.

Contacting the chosen biological sample, or a portion thereof, with the protein under conditions effective and for a period of time sufficient to allow the formation of immune complexes (primary immune complexes). Generally, complex formation is a matter of simply adding the composition to the biological sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or FSGM, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The protein employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the monitoring of efficacy of FXN replacement therapy, e.g., treatment with an FXN fusion protein. Here, a biological or clinical sample suspected of containing either the encoded protein or peptide or corresponding antibody is used. However, these embodiments also have applications to non-clinical samples, such as in the titering of antigen or antibody samples, in the selection of hybridomas, and the like.

The present invention, in particular, contemplates the use of ELISAs as a type of immunodetection assay. It is contemplated that FSGMs of the invention, including one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2, will find utility as immunogens in ELISA assays in monitoring of FXN replacement therapy Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like also may be used.

In one exemplary ELISA, antibodies binding to the FSGMs of the invention, including one or more of Timp1, LRRK2, BASP1, CD44, Xpo6, Lgals3, SOD2, PSMB9, FTL and TXNIP, alone or in combination with one or more additional FSGMs listed in Tables 2, 3, 4 and/or 6, e.g., one or more of CTSS, BTG2, EGR1 and PTGS2, are immobilized onto a selected surface exhibiting protein affinity, such as a well in a polystyrene microtiter plate. Then, a test composition suspected of containing the FSGM antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the FSGM antigen are immobilized onto the well surface and then contacted with the anti-biomarker antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

The phrase “under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

The proteins encoded by one or more of FSGMs of the invention, e.g., one or more of Timp1, LRRK2, BASP1, CD44, Xpo6, Lgals3, SOD2, PSMB9, FTL and TXNIP, can also be measured, quantitated, detected, and otherwise analyzed using protein mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

Whole protein mass analysis of proteins encoded by one or more of FSGMs of the invention, e.g., one or more of Timp1, LRRK2, BASP1, CD44, Xpo6, Lgals3, SOD2, PSMB9, FTL, TXNIP, can be conducted using time-of-flight (TOF) MS, or Fourier transform ion cyclotron resonance (FT-ICR). These two types of instruments are useful because of their wide mass range, and in the case of FT-ICR, its high mass accuracy. The most widely used instruments for peptide mass analysis are the MALDI time-of-flight instruments as they permit the acquisition of peptide mass fingerprints (PMFs) at high pace (1 PMF can be analyzed in approx. 10 sec). Multiple stage quadrupole-time-of-flight and the quadrupole ion trap also find use in this application.

The proteins encoded by one or more of FSGMs of the invention, e.g., one or more of Timp1, LRRK2, BASP1, CD44, Xpo6, Lgals3, SOD2, PSMB9, FTL and TXNIP, can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Gel spots identified on a 2D Gel are usually attributable to one protein. If the identity of the protein is desired, usually the method of in-gel digestion is applied, where the protein spot of interest is excised, and digested proteolytically. The peptide masses resulting from the digestion can be determined by mass spectrometry using peptide mass fingerprinting. If this information does not allow unequivocal identification of the protein, its peptides can be subject to tandem mass spectrometry for de novo sequencing.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

The proteins encoded by one or more of FSGMs of the invention, e.g., one or more of Timp1, LRRK2, BASP1, CD44, Xpo6, Lgals3, SOD2, PSMB9, FTL and TXNIP, can be identified using MS using a variety of techniques, all of which are contemplated herein. Peptide mass fingerprinting uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample. It will be further appreciated that the development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997).

Several recent methods allow for the quantitation of proteins by mass spectrometry. For example, stable (e.g., non-radioactive) heavier isotopes of carbon (13C) or nitrogen (15N) can be incorporated into one sample while the other one can be labeled with corresponding light isotopes (e.g. 12C and 14N). The two samples are mixed before the analysis. Peptides derived from the different samples can be distinguished due to their mass difference. The ratio of their peak intensities corresponds to the relative abundance ratio of the peptides (and proteins). The most popular methods for isotope labeling are SILAC (stable isotope labeling by amino acids in cell culture), trypsin-catalyzed 180 labeling, ICAT (isotope coded affinity tagging), iTRAQ (isobaric tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument. Other types of “label-free” quantitative mass spectrometry, uses the spectral counts (or peptide counts) of digested proteins as a means for determining relative protein amounts.

2. Detection of Nucleic Acids Corresponding to Protein FSGMs

In certain embodiments, the invention involves the detection of nucleic acid FSGMs, e.g., the corresponding genes or mRNA of the FSGMs of the invention, e.g., FSGMs listed in Table 1 and/or Table 5, alone or in combination with one or more additional FSGMs listed in Tables 2, 3, 4 and/or 6, including one or more of Timp1, LRRK2, BASP1, CD44, Xpo6, Lgals3, SOD2, PSMB9, FTL and TXNIP.

In various embodiments, the methods of the present invention generally involve the determination of expression levels of a set of genes in a biological sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes.

For detecting nucleic acids encoding FSGMs of the invention, e.g., Timp1, LRRK2, BASP1, CD44, Xpo6, Lgals3, SOD2, PSMB9, FTL and TXNIP, any suitable method can be used, including, but not limited to, Southern blot analysis, Northern blot analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds), 1990, Academic Press: New York), reverse transcriptase PCR (RT-PCT), quantitative PCR (qPCR), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan®, etc.

In other embodiments, gene expression levels of FSGMs of interest, e.g., Timp1, LRRK2, BASP1, CD44, Xpo6, Lgals3, SOD2, PSMB9, FTL and TXNIP, may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state mRNA level of a large number of genes simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Nucleic acid used as a template for amplification can be isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to any of the FSGM nucleotide sequences identified herein are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994). Following detection, one may compare the results seen in a given subject with a statistically significant reference group of, for example, normal subjects. In this way, it is possible to correlate the amount of nucleic acid detected with various clinical states.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

In PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-la-thiol-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other contemplated nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al. (1989), incorporated herein by reference in its entirety.

Oligonucleotide probes or primers of the present invention may be of any suitable length, depending on the particular assay format and the particular needs and targeted sequences employed. In a preferred embodiment, the oligonucleotide probes or primers are at least 10 nucleotides in length (preferably, 10, 11, 12, 13, 14, 15, 16, 17, 18, 19, 20, 21, 22, 23, 24, 25, 26, 27, 28, 29, 30, 31, 32, or more nucleotides in length) and they may be adapted to be especially suited for a chosen nucleic acid amplification system and/or hybridization system used. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 12 and 30 nucleotides in length are usually designed and used in the art. As well known in the art, probes ranging from 10 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, etc.) are also within the scope of the present invention.

In other embodiments, the detection means the use of a hybridization technique, e.g., where a specific primer or probe is selected to anneal to a target FSGM of interest, and thereafter detection of selective hybridization is made. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.).

To enable hybridization to occur under the assay conditions of the present invention, oligonucleotide primers and probes should comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of an FSGM of the invention. Probes and primers of the present invention are those that hybridize under stringent hybridization conditions and those that hybridize to FSGM homologs of the invention under at least moderately stringent conditions. In certain embodiments, probes and primers of the present invention have complete sequence identity to the FSGMs of the invention (gene sequences (e.g., cDNA or mRNA). It should be understood that other probes and primers could be easily designed and used in the present invention based on the FSGMs of the invention disclosed herein by using methods of computer alignment and sequence analysis known in the art (cf. Molecular Cloning: A Laboratory Manual, Third Edition, edited by Cold Spring Harbor Laboratory, 2000).

3. Antibodies and Labels

In some embodiments, the invention provides methods and compositions that include labels for the highly sensitive detection and quantitation of the FSGMs, e.g., Timp1, LRRK2, BASP1, CD44, Xpo6, Lgals3, SOD2, PSMB9, FTL and TXNIP, of the invention. One skilled in the art will recognize that many strategies can be used for labeling target molecules to enable their detection or discrimination in a mixture of particles. The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner that binds to the FSGM of interest, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

In some embodiments, the invention provides a label for detecting a biological molecule comprising a binding partner for the biological molecule that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. In some embodiments, the biological molecule is a protein or a small molecule. In some embodiments, the biological molecule is a protein. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g., an antibody.

In some embodiments, the invention provides a label for the detection of a biological FSGM of the invention, wherein the label comprises a binding partner for the FSGM and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an antibody. In some embodiments, the antibody is a monoclonal antibody. In other embodiments, the antibody is a polyclonal antibody.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antigen-binding fragment” of an antibody refers to the part of the antibody that participates in antigen binding. The antigen binding site is formed by amino acid residues of the N-terminal variable (“V”) regions of the heavy (“H”) and light (“L”) chains. It will be appreciated that the choice of epitope or region of the molecule to which the antibody is raised will determine its specificity, e.g., for various forms of the molecule, if present, or for total (e.g., all, or substantially all of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Antibodies may be prepared by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g. bacteria) or eukaryotic cells (e.g. yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent VH::VL heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein FSGMs disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Antibodies that bind to the protein FSGMs employed in the present methods are, in some cases, available commercially or can be obtained without undue experimentation.

In still other embodiments, particularly where oligonucleotides are used as binding partners to detect and hybridize to mRNA FSGMs or other nucleic acid based FSGMs, the binding partners (e.g., oligonucleotides) can comprise a label, e.g., a fluorescent moiety or dye. In addition, any binding partner of the invention, e.g., an antibody, can also be labeled with a fluorescent moiety. The fluorescence of the moiety will be sufficient to allow detection in a single molecule detector, such as the single molecule detectors described herein. A “fluorescent moiety,” as that term is used herein, includes one or more fluorescent entities whose total fluorescence is such that the moiety may be detected in the single molecule detectors described herein. Thus, a fluorescent moiety may comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., an FSGM, at a limit of detection of less than about 10, 5, 4, 3, 2, 1, 0.1, 0.01, 0.001, 0.00001, or 0.000001 pg/ml and with a coefficient of variation of less than about 20, 15, 14, 13, 12, 11, 10, 9, 8, 7, 6, 5, 4, 3, 2, 1% or less, e.g., about 10% or less, in the instruments described herein. In some embodiments, the fluorescence of the fluorescent moiety is such that it allows detection and/or quantitation of a molecule, e.g., an FSGM, at a limit of detection of less than about 5, 1, 0.5, 0.1, 0.05, 0.01, 0.005, 0.001 pg/ml and with a coefficient of variation of less than about 10%, in the instruments described herein. “Limit of detection,” or LoD, as those terms are used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

Furthermore, the moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems of the invention (e.g., does not cause precipitation of the protein of interest, or precipitation of a protein to which the moiety has been attached).

Any suitable fluorescent moiety may be used. Examples include, but are not limited to, Alexa Fluor dyes (Molecular Probes, Eugene, Oreg.). The Alexa Fluor dyes are disclosed in U.S. Pat. Nos. 6,977,305; 6,974,874; 6,130,101; and 6,974,305 which are herein incorporated by reference in their entirety. Some embodiments of the invention utilize a dye chosen from the group consisting of Alexa Fluor 647, Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize a dye chosen from the group consisting of Alexa Fluor 488, Alexa Fluor 532, Alexa Fluor 647, Alexa Fluor 700 and Alexa Fluor 750. Some embodiments of the invention utilize a dye chosen from the group consisting of Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize the Alexa Fluor 647 molecule, which has an absorption maximum between about 650 and 660 nm and an emission maximum between about 660 and 670 nm. The Alexa Fluor 647 dye is used alone or in combination with other Alexa Fluor dyes.

In some embodiments, the fluorescent label moiety that is used to detect an FSGM in a sample using the analyzer systems of the invention is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical feature of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

### F. Isolated FSGMs

1. Isolated Polypeptide FSGMs

One aspect of the invention pertains to isolated FSGM proteins and biologically active portions thereof, including Timp1, LRRK2, BASP1, CD44, Xpo6, Lgals3, SOD2, PSMB9, FTL and TXNIP, as well as polypeptide fragments suitable for use as immunogens to raise antibodies directed against an FSGM protein or a fragment thereof. In one embodiment, the native FSGM protein can be isolated by an appropriate purification scheme using standard protein purification techniques. In another embodiment, a protein or peptide comprising the whole or a segment of the FSGM protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of an FSGM protein include polypeptides comprising amino acid sequences sufficiently identical to or derived from the amino acid sequence of the FSGM protein, which include fewer amino acids than the full length protein, and exhibit at least one activity of the corresponding full-length protein. Typically, biologically active portions comprise a domain or motif with at least one activity of the corresponding full-length protein. A biologically active portion of an FSGM protein of the invention can be a polypeptide which is, for example, 10, 25, 50, 100 or more amino acids in length. Moreover, other biologically active portions, in which other regions of the FSGM protein are deleted, can be prepared by recombinant techniques and evaluated for one or more of the functional activities of the native form of the FSGM protein.

Preferred FS GM proteins are listed in Table 1 and/or Table 5, alone or in combination with one or more additional FSGMs listed in Tables 2, 3, 4 and/or 6. Other useful proteins are substantially identical (e.g., at least about 40%, preferably 50%, 60%, 70%, 80%, 90%, 91%, 92%, 93%, 94%, 95%, 96%, 97%, 98% or 99%) to one of these sequences and retain the functional activity of the corresponding naturally-occurring FSGM protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. Preferably, the percent identity between the two sequences is calculated using a global alignment. Alternatively, the percent identity between the two sequences is calculated using a local alignment. The percent identity between the two sequences is a function of the number of identical positions shared by the sequences (i.e., % identity=# of identical positions/total # of positions (e.g., overlapping positions)×100). In one embodiment the two sequences are the same length. In another embodiment, the two sequences are not the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score=50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res. 25:3389-3402, which is able to perform gapped local alignments for the programs BLASTN, BLASTP and BLASTX. Alternatively, PSI-Blast can be used to perform an iterated search which detects distant relationships between molecules. When utilizing BLAST, Gapped BLAST, and PSI-Blast programs, the default parameters of the respective programs (e.g., BLASTX and BLASTN) can be used. See the NCBI website. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

2. Isolated Nucleic Acid FSGMs

One aspect of the invention pertains to isolated nucleic acid molecules which encode an FSGM protein or a portion thereof. Isolated nucleic acids of the invention also include nucleic acid molecules sufficient for use as hybridization probes to identify FSGM nucleic acid molecules, and fragments of FSGM nucleic acid molecules, e.g., those suitable for use as PCR primers for the amplification of a specific product or mutation of FSGM nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule (preferably a protein-encoding sequences) is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N Y, 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of an FSGM nucleic acid or to the nucleotide sequence of a nucleic acid encoding an FSGM protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises an FSGM nucleic acid or which encodes an FSGM protein. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 15, more preferably at least about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more FSGMs of the invention. In certain embodiments, the probes hybridize to nucleic acid sequences that traverse splice junctions. The probe comprises a label group attached thereto, e.g., a radioisotope, a fluorescent compound, an enzyme, or an enzyme co-factor. Such probes can be used as part of a diagnostic test kit or panel for identifying cells or tissues which express or mis-express the protein, such as by measuring levels of a nucleic acid molecule encoding the protein in a sample of cells from a subject, e.g., detecting mRNA levels or determining whether a gene encoding the protein or its translational control sequences have been mutated or deleted.

The invention further encompasses nucleic acid molecules that differ, due to degeneracy of the genetic code, from the nucleotide sequence of nucleic acids encoding an FSGM protein (e.g., protein having the sequence provided in the sequence listing), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to an FSGM of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to an FSGM nucleic acid or to a nucleic acid encoding a marker protein. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6×sodium chloride/sodium citrate (SSC) at about 45° C., followed by one or more washes in 0.2×SSC, 0.1% SDS at 50-65° C.

### G. Frataxin Replacement Therapy

In some aspects, the methodology provided in the present disclosure refers to the determination of a gene expression profile associated with FXN replacement therapy. FXN replacement therapy involves the administration of a therapeutic compound capable of increasing levels of FXN, e.g., an FXN replacement therapeutic, to a subject in need thereof. A number of alternatives for delivery of exogenous FXN, or for otherwise increasing FXN levels in a subject, e.g., an FXN deficient subject, may be envisioned and are encompassed by the present disclosure. The FXN therapy, e.g., FXN replacement therapeutic, may be provided by FXN protein delivery or through delivery of a nucleic acid capable of increasing FXN levels, expression or activity in a subject, e.g., a nucleic acid (e.g., DNA or mRNA) encoding FXN. FXN protein delivery may be delivery of full length FXN or delivery of a FXN fusion protein.

In some embodiments, the FXN replacement therapy comprises administration to a subject in need thereof a nucleic acid capable of increasing levels, expression, and/or activity of FXN in the subject. In some embodiments, the nucleic acid may be a nucleic acid encoding FXN, e.g., FXN mRNA. In some embodiments, the nucleic acid may be an antisense oligonucleotide, e.g., an antisense oligonucleotide that activates expression of FXN protein as described, e.g., in Li et al., Nucleic Acid Ther. 2018, 28(1): 23-33 or in Mikaeili et al., Scientific Reports 2018, 8:17217, the entire contents of each of which are hereby incorporated herein by reference. In some embodiments, the nucleic acid may be siRNA capable of activating expression of FXN protein as described, e.g., in Shen et al., Bioorganic & Medicinal Chemistry Letters 2018, 28(17):2850-2855, the entire contents of which are hereby incorporated herein by reference.

In some embodiments, a nucleic acid capable of increasing levels, expression and/or activity of FXN in a subject may be delivered to a subject, e.g., an FXN deficient subject, via a viral vector.

In some embodiments, the FXN replacement therapy comprises administration of an FXN fusion protein. As used herein, the term “FXN fusion protein” refers to FXN or a fragment of FXN fused to a full length or a fragment of a different protein, or to a peptide. In some embodiments, an FXN fusion protein comprises a polypeptide that comprises FXN, e.g., full-length hFXN (SEQ ID NO: 1) or mature hFXN (SEQ ID NO: 2). In some embodiments, the FXN fusion protein also comprises a cell penetrating peptide (CPP). Exemplary FXN fusion proteins are described, for example, in US 2021/0047378, the entire contents of which are incorporated by reference herein.

The term “cell penetrating peptide” or “CPP”, as used herein, refers to a short peptide sequence, typically between 5 and 30 amino acids long, that can facilitate cellular intake of various molecular cargo, such as proteins. Within the context of the present invention, a CPP present in an FXN fusion protein facilitates the delivery of the FXN fusion protein into a cell, e.g., a recipient cell. CPPs may be polycationic, i.e., have an amino acid composition that either contains a high relative abundance of positively charged amino acids, such as lysine or arginine. CCPs may also be amphipathic, i.e., have sequences that contain an alternating pattern of polar/charged amino acids and non-polar, hydrophobic amino acids. CPPs may also be hydrophobic, i.e., contain only apolar residues with low net charge, or have hydrophobic amino acid groups that are crucial for cellular uptake.

A CPP that may be comprised in the FXN fusion protein may be any CPP known to a person skilled in the art. For example, the CPP may be any CPP listed in the Database of Cell-Penetrating Peptides CPPsite 2.0, the entire contents of which are hereby incorporated herein by reference. For examples, CPPs useful in the context of the present invention may a cell penetrating peptide derived from a protein selected from the group consisting of HIV Trans-Activator of Transcription peptide (HIV-TAT), galanin, mastoparan, transportan, penetratin, polyarginine, VP22, transportan, amphipathic peptides such as MAP, KALA, ppTG20, proline-rich peptides, MPG-derived peptides, Pep-1, and also oligomers, arginine-rich peptides and calcitonin-derived peptides.

In some embodiments, the CPP comprises a TAT protein domain comprising amino acids 47-57 of the 86 amino acid full length HIV-TAT protein (which 11 amino acid peptide may also be referred to herein as “HIV-TAT”; SEQ ID NO:4). In one embodiment, the CPP consists of HIV-TAT (SEQ ID NO:4). In some embodiments, the CPP comprises amino acids 47-57 of the 86 amino acid full length HIV-TAT protein with a methionine added at the amino terminus for initiation (12 AA; “HIV-TAT+M”): MYGRKKRRQRRR (SEQ ID NO: 5). Table 8 below lists amino acid sequences of exemplary CPPs.

TABLE 8
Exemplary CPPs and corresponding sequences

| SEQ ID | | |
|---|---|---|
| NO. | CPP | Amino Acid Sequence |
| 4 | HIV-TAT | YGRKKRRQRRR |
| 5 | HIV-TAT+M | MYGRKKRRQRRR |
| 6 | Galanin | GWTLNSAGYLLGPHAVGN |
| | | HRSFSDKNGLTS |
| 7 | Mastoparan | INLKALAALAKKIL-NH₂ |
| 8 | Transportan | GWTLNSAGYLLGKINLKA |
| | | LAALAKKIL |
| 9 | Penetratin | RQIKIWFQNRRMKWKK |
| 10 | Polyarginine | RRRRRRRRR |
| 11 | VP22 | DAATATRGRSAASRPTERPR |
| | | APARSASRPRRPVE |

In some embodiments, the CPP comprised in the FXN fusion protein is HIV-TAT (SEQ ID NO: 4). In some embodiments, the FXN fusion protein comprises full-length FXN, e.g., SEQ ID NO: 1, and HIV-TAT, e.g., SEQ ID NO: 4, as CPP.

In some embodiments, in FXN fusion proteins of the present disclosure, the CPP may be fused together with the FXN, e.g., full-length FXN, via a linker to form a single polypeptide chain. Examples of FXN fusion proteins include TAT-FXN fusion proteins, where TAT or a fragment of TAT may be directly or indirectly (through a linker) linked to either the N- or the C-terminus of FXN. In one specific example, the linker may comprise the amino acid sequence GG.

In some aspects, the CPP, e.g., HIV-TAT, that is present in an FXN fusion protein of the present disclosure facilitates delivery of the FXN fusion protein into a cell, e.g., a cell that may be present in vitro, ex vivo, or in a subject. Once inside the cell, the FXN fusion protein may be processed by cellular machinery to remove the CPP, e.g., HIV-TAT, from the FXN.

One specific example of a TAT-FXN fusion protein is referred to as an exemplary FXN fusion protein. The exemplary FXN fusion protein is a 24.9 kDa fusion protein currently under investigation as an FXN replacement therapy to restore functional levels of FXN in the mitochondria of FRDA subjects. The exemplary FXN fusion protein includes the HIV-TAT peptide linked to the N-terminus of the full-length hFXN protein. The mechanism of action of the exemplary FXN fusion protein relies on the cell-penetrating ability of the HIV-TAT peptide to deliver the exemplary FXN fusion protein into cells and the subsequent processing into mature hFXN after translocation into the mitochondria. The exemplary FXN fusion protein is described in US 2021/0047378, the entire contents of which are hereby incorporated herein by reference. The exemplary FXN fusion protein comprises the following amino acid sequence (224 amino acids):

(SEQ ID NO: 12)
MYGRKKRRQRRRGGMWTLGRRAVAGLLASPSPAQAQTLTRV
PRPAELAPLCGRRGLRTDIDATCTPRRASSNORGLNQIWNV
KKQSVYLMNLRKSGTLGHPGSLDETTYERLAEETLDSLAEF
FEDLADKPYTFEDYDVSFGSGVLTVKLGGDLGTYVINKQTP
NKQIWLSSPSSGPKRYDWTGKNWVYSHDGVSLHELLAAELT
KALKTKLDLSSLAYSGKDA.

FXN replacement may also be delivered by viral gene replacement, which may utilize retroviral, lentiviral, and adeno-associated viral vectors, as well as adenoviruses. Alternatively, FXN replacement therapy may be achieved by upregulation of the endogenous mutant FXN gene, which depending on the number of GAA repeats is expressed in varying levels in carriers of the mutant FXN allele. Upregulation of the endogenous mutant FXN gene can be achieved by using, for example, antisense oligonucleotides or siRNAs.

### H. Methods of Use of FSGMs

In some aspects, the present disclosure provides methods for evaluating FXN deficiency or monitoring or evaluating progression of FXN deficiency in a subject over time. In these methods the amount of one or more FSGMs described herein, i.e., one or more of the FSGMs listed in Table 1 and/or Table 5, alone or in combination with one or more additional FSGMs listed in Tables 2, 3, 4 and/or 6 (e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2), in a pair of samples obtained from a subject is assessed. The pair of samples may comprise a first sample obtained from the subject at an earlier time point and a second sample obtained from the subject at a later time point. It is understood that the methods of the disclosure include obtaining and analyzing more than two samples (e.g., 3, 4, 5, 6, 7, 8, 9, or more samples) at regular or irregular intervals for assessment of FSGM levels. Pairwise comparisons can be made between consecutive or non-consecutive subject samples. Trends of FSGM levels and rates of change of FSGM levels can be analyzed for any two or more consecutive or non-consecutive subject samples.

Accordingly, the present disclosure provides a method for evaluating an FXN deficiency in a subject, comprising (a) determining an FXN expression profile for one or more FSMGs in a sample obtained from a subject, e.g., an FXN deficient subject or a subject suspected of having an FXN deficiency, wherein the one or more FSGMs comprises one or more markers defined in Table 1 and/or Table 5, alone or in combination with one or more additional FSGMs listed in Tables 2, 3, 4 and/or 6; and (b) comparing the FXN replacement expression profile determined in step (a) with a reference FXN expression profile for the one or more FSGMs; and making a determination about the FXN deficiency of the subject based on the comparison in step (b). In one embodiment, the one or more FSGM comprises one or any combination of Timp1, LRRK2, BASP1, CD44, Xpo6, Lgals3, SOD2, PSMB9, FTL and TXNIP. In another embodiment, the one or more FSGM comprises one or more of Timp1, LRRK2, BASP1, CD44, Xpo6, Lgals3, SOD2, PSMB9, FTL and TXNIP in combination with one or more additional FSGMs in Tables 2, 3, 4 and/or 6, e.g., one or more of CTSS, BTG2, EGR1 and PTGS2. In one embodiment, a determination as to the severity of the FXN deficiency is made. In one embodiment, the one or more FSGMs comprise or consist of LRRK2 and Xpo6. In one embodiment, the one or more FSGMs comprise or consist of LRRK2, Xpo6, CTSS, BTG2, EGR1 and PTGS2.

i. Methods for Monitoring Progression of FXN Deficiency

The present disclosure further provides a method for monitoring progression of an FXN deficiency in a subject, the method comprising: (a) determining a first FXN expression profile for one or more FSGMs in a first sample obtained from a subject, e.g., an FXN deficient subject or a subject suspected of having an FXN deficiency, at a first time point, wherein the one or more FSGMs comprises one or more markers defined in Table 1 and/or Table 5, alone or in combination with one or more additional FSGMs listed in Tables 2, 3, 4 and/or 6; (b) determining a second FXN expression profile for the one or more FSGMs in a second sample obtained from the subject at a second time point that is later than the first time point; and (c) comparing the second FXN expression profile with the first FXN profile; thereby monitoring FXN deficiency in the subject. In one embodiment, the one or more FSGM comprises one or any combination of Timp1, LRRK2, BASP1, CD44, Xpo6, Lgals3, SOD2, PSMB9, FTL and TXNIP. In another embodiment, the one or more FSGM comprises one or more of Timp1, LRRK2, BASP1, CD44, Xpo6, Lgals3, SOD2, PSMB9, FTL and TXNIP, alone or in combination with one or more additional FSGMs in Tables 2, 3, 4 and/or 6, e.g., one or more of CTSS, BTG2, EGR1 and PTGS2. In one embodiment, the one or more FSGMs comprise or consist of LRRK2, Xpo6, CTSS, BTG2, EGR1 and PTGS2.

In some embodiments, a decrease over time in the amount of any one or more of Timp, LRRK2, BASP1, CD44, XPO6, Lgals3, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in a sample obtained from an FXN deficient subject is indicative that the FXN deficiency has progressed.

In some embodiments, a decrease in the amount of Timp1 in the second FXN expression profile as compared to the first FXN expression profile is indicative that the FXN deficiency has progressed. In some embodiments, a decrease in the amount of LRRK2 in the second FXN expression profile as compared to the first FXN expression profile is indicative that the FXN deficiency has progressed. In some embodiments, a decrease in the amount of BASP1 in the second FXN expression profile as compared to the first FXN expression profile is indicative that the FXN deficiency has progressed. In some embodiments, a decrease in the amount of CD44 in the second FXN expression profile as compared to the first FXN expression profile is indicative that the FXN deficiency has progressed. In some embodiments, a decrease in the amount of Xpo6 in the second FXN expression profile as compared to the first FXN expression profile is indicative that the FXN deficiency has progressed. In some embodiments, an increase in the amount of Lgals3 in the second FXN expression profile as compared to the first FXN expression profile is indicative that the FXN deficiency has progressed. In some embodiments, a decrease in the amount of SOD2 in the second FXN expression profile as compared to the first FXN expression profile is indicative that the FXN deficiency has progressed. In some embodiments, a decrease in the amount of PSMB9 in the second FXN expression profile as compared to the first FXN expression profile is indicative that the FXN deficiency has progressed. In some embodiments, a decrease in the amount of FTL in the second FXN expression profile as compared to the first FXN expression profile is indicative that the FXN deficiency has progressed. In some embodiments, a decrease in the amount of TXNIP in the second FXN expression profile as compared to the first FXN expression profile is indicative that the FXN deficiency has progressed. In some embodiments, a decrease in the amount of EGR1 in the second FXN expression profile as compared to the first FXN expression profile is indicative that the FXN deficiency has progressed. In some embodiments, a decrease in the amount of CTSS in the second FXN expression profile as compared to the first FXN expression profile is indicative that the FXN deficiency has progressed. In some embodiments, a decrease in the amount of BTG2 in the second FXN expression profile as compared to the first FXN expression profile is indicative that the FXN deficiency has progressed. In some embodiments, a decrease in the amount of PTGS2 in the second FXN expression profile as compared to the first FXN expression profile is indicative that the FXN deficiency has progressed. In some embodiments, a decrease in the amount of LY96 in the second FXN expression profile as compared to the first FXN expression profile is indicative that the FXN deficiency has progressed. In some embodiments, a decrease in the amount of Adgre1 in the second FXN expression profile as compared to the first FXN expression profile is indicative that the FXN deficiency has progressed. In some embodiments, a decrease in the amount of PSMB8 in the second FXN expression profile as compared to the first FXN expression profile is indicative that the FXN deficiency has progressed. In some embodiments, a decrease in the amount of C3ar1 in the second FXN expression profile as compared to the first FXN expression profile is indicative that the FXN deficiency has progressed. In some embodiments, a decrease in the amount of TFRC in the second FXN expression profile as compared to the first FXN expression profile is indicative that the FXN deficiency has progressed. In some embodiments, a decrease in the amount of PLCL2 in the second FXN expression profile as compared to the first FXN expression profile is indicative that the FXN deficiency has progressed. In some embodiments, a decrease in the amount of S100a4 in the second FXN expression profile as compared to the first FXN expression profile is indicative that the FXN deficiency has progressed. In some embodiments, a decrease in the amount of Mapre1 in the second FXN expression profile as compared to the first FXN expression profile is indicative that the FXN deficiency has progressed. In some embodiments, a decrease in the amount of CFH in the second FXN expression profile as compared to the first FXN expression profile is indicative that the FXN deficiency has progressed. In some embodiments, a decrease in the amount of Tmem70 in the second FXN expression profile as compared to the first FXN expression profile is indicative that the FXN deficiency has progressed. In some embodiments, a decrease in the amount of any two or more of the foregoing FSMGs (e.g., 3, 4, 5, 6, 7, 8, 9, 10, 15, 20 or more) in the second FXN expression profile as compared to the first FXN expression profile is indicative that the FXN deficiency has progressed.

In some embodiments, an increase in the amount of Ube2v2 in the second FXN expression profile as compared to the first FXN expression profile is indicative that the FXN deficiency has progressed. In some embodiments, an increase in the amount of Sec61g in the second FXN expression profile as compared to the first FXN expression profile is indicative that the FXN deficiency has progressed. In some embodiments, an increase in the amount of Zdhhc13 in the second FXN expression profile as compared to the first FXN expression profile is indicative that the FXN deficiency has progressed. In some embodiments, an increase in the amount of Yipf5 in the second FXN expression profile as compared to the first FXN expression profile is indicative that the FXN deficiency has progressed. In some embodiments, an increase in the amount of Tpp2 in the second FXN expression profile as compared to the first FXN expression profile is indicative that the FXN deficiency has progressed. In some embodiments, an increase in the amount of Ybx2 in the second FXN expression profile as compared to the first FXN expression profile is indicative that the FXN deficiency has progressed. In some embodiments, an increase in the amount of Ptms in the second FXN expression profile as compared to the first FXN expression profile is indicative that the FXN deficiency has progressed. In some embodiments, an increase in the amount of any two or more of the foregoing FSMGs (e.g., 3, 4, 5, 6, 7, 8, 9, 10, or more) in the second FXN expression profile as compared to the first FXN expression profile is indicative that the FXN deficiency has progressed.

In some embodiments, lack of a decrease in the amount of Timp1 in the second FXN expression profile as compared to the first FXN expression profile is indicative that the FXN deficiency has not progressed. In some embodiments, lack of a decrease in the amount of LRRK2 in the second FXN expression profile as compared to the first FXN expression profile is indicative that the FXN deficiency has not progressed. In some embodiments, lack of a decrease in the amount of BASP1 in the second FXN expression profile as compared to the first FXN expression profile is indicative that the FXN deficiency has not progressed. In some embodiments, lack of a decrease in the amount of CD44 in the second FXN expression profile as compared to the first FXN expression profile is indicative that the FXN deficiency has not progressed. In some embodiments, lack of a decrease in the amount of Xpo6 in the second FXN expression profile as compared to the first FXN expression profile is indicative that the FXN deficiency has not progressed. In some embodiments, lack of an increase in the amount of Lgals3 in the second FXN expression profile as compared to the first FXN expression profile is indicative that the FXN deficiency has not progressed. In some embodiments, lack of a decrease in the amount of SOD2 in the second FXN expression profile as compared to the first FXN expression profile is indicative that the FXN deficiency has not progressed. In some embodiments, lack of a decrease in the amount of PSMB9 in the second FXN expression profile as compared to the first FXN expression profile is indicative that the FXN deficiency has not progressed. In some embodiments, lack of a decrease in the amount of FTL in the second FXN expression profile as compared to the first FXN expression profile is indicative that the FXN deficiency has not progressed. In some embodiments, lack of a decrease in the amount of TXNIP in the second FXN expression profile as compared to the first FXN expression profile is indicative that the FXN deficiency has not progressed. In some embodiments, lack of a decrease in the amount of EGR1 in the second FXN expression profile as compared to the first FXN expression profile is indicative that the FXN deficiency has not progressed. In some embodiments, lack of a decrease in the amount of CTSS in the second FXN expression profile as compared to the first FXN expression profile is indicative that the FXN deficiency has not progressed. In some embodiments, lack of a decrease in the amount of BTG2 in the second FXN expression profile as compared to the first FXN expression profile is indicative that the FXN deficiency has not progressed. In some embodiments, lack of a decrease in the amount of PTGS2 in the second FXN expression profile as compared to the first FXN expression profile is indicative that the FXN deficiency has not progressed. In some embodiments, lack of a decrease in the amount of LY96 in the second FXN expression profile as compared to the first FXN expression profile is indicative that the FXN deficiency has not progressed. In some embodiments, lack a decrease in the amount of Adgre1 in the second FXN expression profile as compared to the first FXN expression profile is indicative that the FXN deficiency has not progressed. In some embodiments, lack a decrease in the amount of PSMB8 in the second FXN expression profile as compared to the first FXN expression profile is indicative that the FXN deficiency has not progressed. In some embodiments, lack of a decrease in the amount of C3ar1 in the second FXN expression profile as compared to the first FXN expression profile is indicative that the FXN deficiency has not progressed. In some embodiments, lack of a decrease in the amount of TFRC in the second FXN expression profile as compared to the first FXN expression profile is indicative that the FXN deficiency has not progressed. In some embodiments, lack of a decrease in the amount of PLCL2 in the second FXN expression profile as compared to the first FXN expression profile is indicative that the FXN deficiency has not progressed. In some embodiments, lack of a decrease in the amount of S100a4 in the second FXN expression profile as compared to the first FXN expression profile is indicative that the FXN deficiency has not progressed. In some embodiments, lack of a decrease in the amount of Mapre1 in the second FXN expression profile as compared to the first FXN expression profile is indicative that the FXN deficiency has not progressed. In some embodiments, lack of a decrease in the amount of CFH in the second FXN expression profile as compared to the first FXN expression profile is indicative that the FXN deficiency has not progressed. In some embodiments, lack of a decrease in the amount of Tmem70 in the second FXN expression profile as compared to the first FXN expression profile is indicative that the FXN deficiency has not progressed. In some embodiments, lack of a decrease in the amount of Ube2v2 in the second FXN expression profile as compared to the first FXN expression profile is indicative that the FXN deficiency has not progressed. In some embodiments, a lack of a decrease in the amount of any two or more of the foregoing FSMGs (e.g., 3, 4, 5, 6, 7, 8, 9, 10, 15, 20 or more) in the second FXN expression profile as compared to the first FXN expression profile is indicative that the FXN deficiency has not progressed.

In some embodiments, lack of an increase in the amount of Sec61g in the second FXN expression profile as compared to the first FXN expression profile is indicative that the FXN deficiency has not progressed. In some embodiments, lack of an increase in the amount of Zdhhc13 in the second FXN expression profile as compared to the first FXN expression profile is indicative that the FXN deficiency has not progressed. In some embodiments, lack of an increase in the amount of Yipf5 in the second FXN expression profile as compared to the first FXN expression profile is indicative that the FXN deficiency has not progressed. In some embodiments, lack of an increase in the amount of Tpp2 in the second FXN expression profile as compared to the first FXN expression profile is indicative that the FXN deficiency has not progressed. In some embodiments, lack of an increase in the amount of Ybx2 in the second FXN expression profile as compared to the first FXN expression profile is indicative that the FXN deficiency has not progressed. In some embodiments, lack of an increase in the amount of Ptms in the second FXN expression profile as compared to the first FXN expression profile is indicative that the FXN deficiency has not progressed. In some embodiments, a lack of an increase in the amount of any two or more of the foregoing FSMGs (e.g., 3, 4, 5, 6, 7, 8, 9, 10, 15, 20 or more) in the second FXN expression profile as compared to the first FXN expression profile is indicative that the FXN deficiency has not progressed.

ii. Methods for Evaluating Efficacy of a FXN Replacement Therapy

The present disclosure provides methods for evaluating and/or monitoring the efficacy of FXN replacement therapy in a subject. The invention further provides methods for determining whether a subject is in need of FXN replacement therapy or a change in FXN replacement therapy, e.g., determining whether FXN replacement therapy should be initiated, altered (e.g., dosage and/or administration frequency is increased or decreased), or ceased in a subject.

In some aspects, the present disclosure provides a method for evaluating efficacy of a FXN replacement therapy that comprises: (a) determining a baseline FXN(−) expression profile for one or more FXN-sensitive genomic markers (FSGMs) in a sample obtained from an FXN deficient subject prior to administration of the FXN replacement therapy; (b) determining an FXN replacement expression profile for the one or more FXN-sensitive genomic markers (FSGMs) in a sample obtained from the FXN deficient subject following administration of the FXN replacement therapy; (c) comparing the FXN replacement expression profile determined in step (b) with the baseline FXN(−) expression profile determined in step (a); and (d) determining efficacy of the FXN replacement therapy based on the comparison in step (c); wherein the one or more FSGMs are selected from the markers defined in Table 1; or the markers defined in Table 1 and/or Table 5, alone or in combination with one or more additional FSGMs listed in Tables 2, 3, 4 and/or 6. In one embodiment, the one or more FSGM comprises one or any combination of Timp1, LRRK2, BASP1, CD44CD44, Xpo6, Lgals3, SOD2, and PSMB9, FTL and TXNIP. In another embodiment, the one or more FSGM comprises one or more of Timp1, LRRK2, BASP1, CD44CD44, Xpo6, Lgals3, SOD2, and PSMB9, FTL and TXNIP, alone or in combination with one or more additional FSGMs in Tables 2, 3, 4 and/or 6, e.g., one or more of CTSS, BTG2, EGR1 and PTGS2. In one embodiment, the one or more FSGMs comprise or consist of LRRK2 and Xpo6. In one embodiment, the one or more FSGMs comprise or consist of LRRK2, Xpo6, CTSS, BTG2, EGR1 and PTGS2.

In some embodiments, an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from an FXN deficient subject following administration of the FXN replacement therapy as compared to the amount in the sample obtained from an FXN deficient subject prior to administration of the FXN replacement therapy is indicative that the FXN replacement therapy is efficacious.

In some embodiments, a decrease in the amount of any one or more of Lgals3, Ube2v2, Sec61g, Zdhhc13, Yipf5, Tpp2, Ybx2 and/or Ptms in the sample obtained from an FXN deficient subject following administration of the FXN replacement therapy as compared to the amount in the sample obtained from an FXN deficient subject prior to administration of the FXN replacement therapy is indicative that the FXN replacement therapy is efficacious.

In some embodiments, a lack of an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from an FXN deficient subject following administration of the FXN replacement therapy as compared to the amount in the sample obtained from an FXN deficient subject prior to administration of the FXN replacement therapy is indicative that the FXN replacement therapy is not efficacious.

In some embodiments, a lack of a decrease in the amount of any one or more of Lgals3, Ube2v2, Sec61g, Zdhhc13, Yipf5, Tpp2, Ybx2 and/or Ptms in the sample obtained from an FXN deficient subject following administration of the FXN replacement therapy as compared to the amount in the sample obtained from an FXN deficient subject prior to administration of the FXN replacement therapy is indicative that the FXN replacement therapy is not efficacious.

In some aspects, the present disclosure also provides a method for evaluating efficacy of a frataxin (FXN) replacement therapy that comprises: (a) determining an FXN replacement expression profile for one or more FXN-sensitive genomic markers (FSGMs) in a sample obtained from an FXN deficient subject following administration of an FXN replacement therapy; (b) comparing the subject FXN replacement expression profile determined in step (a) with a reference FXN expression profile for the one or more FSGMs; and (c) determining efficacy of the FXN replacement therapy based on the comparison in step (b); wherein the one or more FSGMs are selected from the markers defined in Table 1; or the markers defined in Table 1 and/or Table 5, alone or in combination with one or more additional FSGMs listed in Tables 2, 3, 4 and/or 6. In one embodiment, the one or more FSGM comprises one or any combination of Timp1, LRRK2, BASP1, CD44CD44, Xpo6, Lgals3, SOD2, and PSMB9, FTL and TXNIP. In another embodiment, the one or more FSGM comprises one or more of Timp1, LRRK2, BASP1, CD44CD44, Xpo6, Lgals3, SOD2, and PSMB9, FTL and TXNIP, alone or in combination with one or more additional FSGMs in Tables 2, 3, 4 and/or 6, e.g., one or more of CTSS, BTG2, EGR1 and PTGS2. In one embodiment, the one or more FSGMs comprise or consist of LRRK2, Xpo6, CTSS, BTG2, EGR1 and PTGS2.

In some embodiments, the reference FXN expression profile is a baseline FXN(−) expression profile for the one or more FSGMs. In some embodiments where the reference FXN expression profile is a baseline FXN(−) expression profile for the one or more FSGMs, an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject following administration of the FXN replacement therapy as compared to the amount in the baseline FXN(−) expression profile (e.g., FXN(−) expression profile for a sample obtained from an FXN deficient subject prior to administration of the FXN replacement therapy) is indicative that the FXN replacement therapy is efficacious.

In some embodiments, wherein the reference FXN expression profile is a baseline FXN(−) expression profile for the one or more FSGMs, a decrease in the amount of any one or more of Lgals3, Ube2v2, Sec61g, Zdhhc13, Yipf5, Tpp2, Ybx2 and/or Ptms in the sample obtained from the FXN deficient subject following administration of the FXN replacement therapy as compared to the amount in the baseline FXN(−) expression profile (e.g., FXN(−) expression profile for sample obtained from an FXN deficient subject prior to administration of the replacement therapy) is indicative that the FXN replacement therapy is efficacious.

In some embodiments, wherein the reference FXN expression profile is a baseline FXN(−) expression profile for the one or more FSGMs, lack of an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from an FXN deficient subject following administration of the FXN replacement therapy as compared to the amount in the baseline FXN(−) expression profile (e.g., FXN(−) expression profile for a sample obtained from an FXN deficient subject prior to administration of the FXN replacement therapy) is indicative that the FXN replacement therapy is not efficacious.

In some embodiments, wherein the reference FXN expression profile is a baseline FXN(−) expression profile for the one or more FSGMs, lack of a decrease in the amount of any one or more of Lgals3, Ube2v2, Sec61g, Zdhhc13, Yipf5, Tpp2, Ybx2 and/or Ptms in the sample obtained from an FXN deficient subject following administration of the FXN replacement therapy as compared to the amount in the baseline FXN(−) expression profile (e.g., FXN(−) expression profile for a sample obtained from an FXN deficient subject prior to administration of the FXN replacement therapy) is indicative that the FXN replacement therapy is not efficacious.

In embodiments where the reference FXN expression profile FXN(−) is a baseline FXN(−) expression profile, the baseline FXN(−) expression profile can be determined for the one or more FSGMs in a sample that is obtained from the same FXN deficient subject to whom the FXN replacement therapy is administered. Alternatively, the baseline FXN(−) expression profile can be determined for the one or more FSGMs in a sample that is obtained from a different FXN deficient subject, i.e., to whom the FXN replacement therapy is not administered. In some embodiments, the baseline FXN(−) expression profile is determined from samples obtained from a plurality of FXN deficient subjects, and can comprise, e.g, an average value for the one or FSMGs based on the measurements from the plurality of subjects.

In other embodiments, the reference FXN expression profile is a normal FXN expression profile.

In some embodiments, wherein the reference FXN expression profile is a normal FXN expression profile for the one or more FSGMs, an amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject following administration of the FXN replacement therapy that is similar to the amount in the normal FXN expression profile is indicative that the FXN replacement therapy is efficacious, and/or that the dose and/or frequency of administration of the FXN replacement therapy should be maintained.

In some embodiments, wherein the reference FXN expression profile is a normal FXN expression profile for the one or more FSGMs, an amount of any one or more of Lgals3, Ube2v2, Sec61g, Zdhhc13, Yipf5, Tpp2, Ybx2 and/or Ptms in the sample obtained from the FXN deficient subject following administration of the FXN replacement therapy that is similar to the amount in the normal FXN expression profile is indicative that the FXN replacement therapy is efficacious, and/or that the dose and/or frequency of administration of the FXN replacement therapy should be maintained. In some embodiments, wherein the reference FXN expression profile is a normal FXN expression profile for the one or more FSGMs, a decreased amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject following administration of the FXN replacement therapy as compared to the amount in the normal FXN expression profile is indicative that the FXN replacement therapy is not efficacious, and/or that the dose and/or frequency of administration of the FXN replacement therapy should be increased.

In some embodiments, wherein the reference FXN expression profile is a normal FXN expression profile for the one or more FSGMs, an increased amount of any one or more of Lgals3, Ube2v2, Sec61g, Zdhhc13, Yipf5, Tpp2, Ybx2 and/or Ptms in the sample obtained from the FXN deficient subject following administration of the FXN replacement therapy as compared to the amount in the normal FXN expression profile is indicative that the FXN replacement therapy is not efficacious, and/or that the dose and/or frequency of administration of the FXN replacement therapy should be increased.

In the foregoing methods, when an FXN replacement therapy is determined not to be efficacious, changes may be made to the FXN replacement therapy regimen in order to improve the effects of the therapy or ultimately to achieve efficacy. For example, administration of a higher dose, administration of the same or similar dose at a greater frequency, or administration of a higher dose at a greater frequency, may improve the outcome of the FXN replacement therapy. Accordingly, in some embodiments of the methods provided herein, the methods further comprise altering an FXN replacement therapy regimen based on the efficacy determination made. In some embodiments of the methods provided herein, the methods further comprise the step of recommending, e.g., to a health care provider, to alter the FXN replacement therapy regimen based on the efficacy determination made. Altering the FXN replacement therapy regimen can include modulating (e.g., increasing or decreasing) the dosage, modulating (e.g., increasing or decreasing) the administration frequency, or both, of the FXN replacement therapy.

iii. Methods for Monitoring Treatment with FXN Replacement Therapy

In some aspects, the present disclosure provides a method of monitoring treatment of a subject with a FXN replacement therapy, that comprises: (a) determining a first FXN replacement expression profile for one or more FXN-sensitive genomic markers (FSGMs) in a first sample obtained from an FXN deficient subject at a first time point following administration of an FXN replacement therapy to the subject, wherein the one or more FSGMs comprises one or more markers defined in Table 1; or the markers defined in Table 1 and/or Table 5, alone or in combination with one or more additional FSGMs listed in Tables 2, 3, 4 and/or 6; (b) determining a second FXN replacement expression profile for the one or more FXN-sensitive genomic markers (FSGMs) in a second sample obtained from the subject at a second time point that is later than the first time point; and (c) comparing the second FXN replacement expression profile with the first FXN replacement profile; thereby monitoring treatment of the subject with the FXN replacement therapy. In one embodiment, the one or more FSGMs comprise or consist of LRRK2 and Xpo6. In one embodiment, the one or more FSGMs comprise or consist of LRRK2, Xpo6, CTSS, BTG2, EGR1 and PTGS2.

In some embodiments, the method further comprises making a determination, or making a recommendation to a healthcare provider, to maintain the FXN replacement therapy regimen based on the comparison in step (c). In some embodiments, the method further comprises making a determination, or making a recommendation to a healthcare provider, to alter the FXN replacement therapy regimen based on the comparison in step (c). In some embodiments, the method further comprises making a determination to maintain the dose and/or administration frequency, increase the dose and/or administration frequency, or decrease the dose and/or administration frequency of the FXN replacement therapy based on the comparison in step (c). In some embodiments, the method further comprises making a recommendation, e.g., to a healthcare provider, to maintain the dose and/or administration frequency, increase the dose and/or administration frequency, or decrease the dose and/or administration frequency of the FXN replacement therapy based on the comparison in step (c).

In some embodiments, the method further comprises continuing to administer the FXN replacement therapy regimen (e.g., without changing the regimen, e.g., maintaining the dose and/or administration frequency) to the subject based on the comparison in step (c). In some embodiments, the method further comprises altering the FXN replacement therapy regimen based on the comparison in step (c). In some embodiments, the method further comprises administering an altered FXN replacement therapy regimen based on the comparison in step (c). In some embodiments, administering an altered FXN replacement therapy regimen comprises administering an increased dose and/or administration frequency, or administering a decreased dose and/or administration frequency of the FXN replacement therapy.

In some embodiments, an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the second sample as compared to the amount in the first sample is indicative that the level of FXN in the subject has increased, and/or that the FXN replacement therapy is efficacious.

In some embodiments, a decrease in the amount of any one or more of Lgals3, Ube2v2, Sec61g, Zdhhc13, Yipf5, Tpp2, Ybx2 and/or Ptms in the second sample as compared to the amount in the first sample is indicative that the level of FXN in the subject has increased, and/or that the FXN replacement therapy is efficacious.

In some embodiments, an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the second sample as compared to the amount in the first sample is indicative that the level of FXN in the subject has increased, and/or that the FXN replacement therapy is efficacious.

In some embodiments, a decrease in the amount of any one or more of Lgals3, Ube2v2, Sec61g, Zdhhc13, Yipf5, Tpp2, Ybx2 and/or Ptms in the second sample as compared to the amount in the first sample is indicative that the level of FXN in the subject has increased, and/or that the FXN replacement therapy is efficacious.

In some embodiments, a lack of an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the second sample as compared to the amount in the first sample is indicative that the level of FXN in the subject has remained constant, or that FXN replacement therapy is not efficacious.

In some embodiments, a lack of a decrease in the amount of any one or more of Lgals3, Ube2v2, Sec61g, Zdhhc13, Yipf5, Tpp2, Ybx2 and/or Ptms in the first sample as compared to the amount in the second sample is indicative that the level of FXN in the subject has remained constant, or that the FXN replacement therapy is not efficacious.

It will be appreciated that following administration of FXN replacement therapy to an FXN deficient subject, the level of FXN achieved in the subject may reach a desired or target level of FXN, e.g., similar to levels of FXN present in a normal, healthy subject, or similar to levels of FXN present in hFXN heterozygotes. In such instances, it is beneficial for the dose and/or frequency of administration of the FXN replacement therapy to then be maintained. Further, when such a desired level of FXN in the subject is achieved and maintained constant over time, it is beneficial for the dose and/or frequency of administration of the FXN replacement therapy to be maintained. It will be further appreciated that following administration of FXN replacement therapy to an FXN deficient subject, the level of FXN achieved in the subject may be higher than the desired or target level of FXN, e.g., higher than the level of FXN present in a normal, healthy subject, or higher than the level of FXN present in hFXN heterozygotes. In such instances, it is beneficial for the dose and/or frequency of administration of the FXN replacement therapy to be decreased. Alternatively, the level of FXN achieved in the subject may be lower than the desired or target level of FXN, e.g., lower than the level of FXN present in a normal, healthy subject, or lower than the level of FXN present in hFXN heterozygotes. In such instances, it is beneficial for the dose and/or frequency of administration of the FXN replacement therapy to be increased.

Accordingly, in some embodiments, an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the second sample as compared to the amount in the first sample is indicative that the dose and/or administration frequency of the FXN replacement therapy should be maintained or decreased.

In some embodiments, a decrease in the amount of any one or more of Lgals3, Ube2v2, Sec61g, Zdhhc13, Yipf5, Tpp2, Ybx2 and/or Ptms in the second sample as compared to the amount in the first sample is indicative that the dose and/or administration frequency of the FXN replacement therapy should be maintained or decreased.

In some embodiments, a lack of an increase in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the second sample as compared to the amount in the first sample is indicative that the dose and/or administration frequency of the FXN replacement therapy should be maintained or increased.

In some embodiments, a lack of a decrease in the amount of any one or more of Lgals3, Ube2v2, Sec61g, Zdhhc13, Yipf5, Tpp2, Ybx2 and/or Ptms in the first sample as compared to the amount in the second sample is indicative that the dose and/or administration frequency of the FXN replacement therapy should be maintained or increased.

iv. Methods for Treating FXN Deficiency

In some aspects, the present disclosure also provides a method for treating an FXN deficiency in a subject that comprises: (a) determining an FXN expression profile in a sample obtained from an FXN deficient subject for one or more FXN-sensitive genomic markers (FSGMs); (b) comparing the FXN expression profile of the sample with at least one other expression profile selected from the group consisting of normal FXN expression profile for the one or more FSGMs, baseline FXN(−) expression profile for the one or more FSGMs, and FXN replacement expression profile for the one or more FSGMs; (c) classifying the FXN expression profile determined in step (a) as corresponding to a normal FXN expression profile, baseline FXN(−) expression profile or an FXN replacement expression profile; and (d) maintaining, initiating or modulating an FXN replacement therapy based on the classification of the FXN expression profile of the sample; wherein the one or more FSGMs comprises one or more markers defined in Table 1; or the markers defined in Table 1 and/or Table 5, alone or in combination with one or more additional FSGMs listed in Tables 2, 3, 4 and/or 6.

In one embodiment, the one or more FSGMs comprise or consist of LRRK2 and Xpo6. In one embodiment, the one or more FSGMs comprise or consist of LRRK2, Xpo6, CTSS, BTG2, EGR1 and PTGS2.

In some embodiments, modulating an FXN replacement therapy comprises increasing the dosage, decreasing the dosage, increasing the administration frequency, decreasing the administration frequency, or any combination thereof, of the FXN replacement therapy.

In some embodiments, when there is a decrease in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject as compared to the amount in the normal FXN expression profile, the FXN expression profile determined in step (a) is classified as corresponding to a baseline FXN(−) expression profile.

In some embodiments, when the FXN expression profile in the sample is classified as a baseline FXN(−) expression profile, administration of an FXN replacement therapy is initiated in the FXN deficient subject. In some embodiments, when the FXN expression profile in the sample is classified as a baseline FXN(−) expression profile, and the FXN deficient subject is already undergoing FXN replacement therapy, the FXN replacement therapy regiment is altered, e.g., the dose and/or administration frequency of the FXN replacement therapy is increased.

In some embodiments, when there is a lack of a change (e.g., lack of a decrease) in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject as compared to the amount in the normal FXN expression profile, the FXN expression profile determined in step (a) is classified as corresponding to a normal FXN expression profile.

In some embodiments, when the FXN expression profile in the sample is classified as a normal FXN expression profile, administration of an FXN replacement therapy is not initiated in the FXN deficient subject. In some embodiments, when the FXN expression profile in the sample is classified as a normal FXN expression profile, and the FXN deficient subject is already undergoing FXN replacement therapy, the FXN replacement therapy regimen is maintained (i.e., not changed), e.g., the dose and/or administration frequency of the FXN replacement therapy is maintained.

In some embodiments, when there is an increase in the amount of any one or more of Lgals3, Ube2v2, Sec61g, Zdhhc13, Yipf5, Tpp2, Ybx2 and/or Ptms in the sample obtained from the FXN deficient subject as compared to the amount in the normal FXN expression profile, i the FXN expression profile determined in step (a) is classified as corresponding to a baseline FXN(−) expression profile.

In some embodiments, when the FXN expression profile in the sample is classified as a baseline FXN(−) expression profile, administration of an FXN replacement therapy is initiated in the FXN deficient subject. In some embodiments, when the FXN expression profile in the sample is classified as a baseline FXN(−) expression profile, and the FXN deficient subject is already undergoing FXN replacement therapy, the FXN replacement therapy regiment is altered, e.g., the dose and/or administration frequency of the FXN replacement therapy is increased

In some embodiments, when there is no change (e.g., lack of an increase) in the amount of any one or more of Lgals3, Ube2v2, Sec61g, Zdhhc13, Yipf5, Tpp2, Ybx2 and/or Ptms in the sample obtained from the FXN deficient subject as compared to the amount in the normal FXN expression profile, the FXN expression profile determined in step (a) is classified as corresponding to a normal FXN expression profile.

In some embodiments, when the FXN expression profile in the sample is classified as corresponding to a normal FXN expression profile, administration of an FXN replacement therapy is not initiated in the FXN deficient subject. In some embodiments, when the FXN expression profile in the sample is classified as a normal FXN expression profile, and the FXN deficient subject is already undergoing FXN replacement therapy, the FXN replacement therapy regimen is maintained (i.e., not changed), e.g., the dose and/or administration frequency of the FXN replacement therapy is maintained.

In some embodiments, modulating an FXN replacement therapy comprises increasing the dosage, decreasing the dosage, increasing the administration frequency, decreasing the administration frequence, or any combination thereof, of the FXN replacement therapy.

In some aspects, the present disclosure provides a method of treating an FXN deficiency in a subject that comprises: (a) determining an FXN expression profile for one or more FSGMs in a sample from an FXN deficient subject; and (b) recommending to a healthcare provider to administer an FXN replacement therapy to the subject based on the subject FXN expression profile determined in step (a). In some aspects, the present disclosure also provides a method of treating an FXN deficiency in a subject that comprises: (a) obtaining an FXN expression profile for one or more FSGMs in a sample obtained from an FXN deficient subject; and (b) administering an FXN replacement therapy to the subject based on the subject FXN expression profile. In some embodiments, the one or more one or more FSGMs comprises one or more markers defined in Table 1; or the markers defined in Table 1 and/or Table 5, alone or in combination with one or more additional FSGMs listed in Tables 2, 3, 4 and/or 6.

In one embodiment, the one or more FSGMs comprise or consist of LRRK2 and Xpo6. In one embodiment, the one or more FSGMs comprise or consist of LRRK2, Xpo6, CTSS, BTG2, EGR1 and PTGS2.

In some embodiments, the method further comprises comparing the FXN expression profile for the one or more FSGMs in the sample with at least one other expression profile selected from the group consisting of normal FXN expression profile for the one or more FSGMs, baseline FXN(−) expression profile for the one or more FSGMs, and FXN replacement expression profile for the one or more FSGMs. In some embodiments, the method further comprises classifying the FXN expression profile for the one or more FSGMs in the sample as corresponding to a normal FXN expression profile for the one or more FSGMs, baseline FXN(−) expression profile for the one or more FSGMs, or FXN replacement expression profile for the one or more FSGMs.

In some embodiments, when there is a decrease in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject as compared to the amount in the normal FXN expression profile, then an FXN replacement therapy is administered to the FXN deficient subject, or a recommendation to a healthcare provider is made to administer an FXN replacement therapy to the FXN deficient subject. In some embodiments, when there is a decrease in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject as compared to the amount in the normal FXN expression profile, and the FXN deficient subject is undergoing an FXN replacement therapy, then the FXN replacement therapy regimen is altered (e.g. the dosage and/or administration frequency is increased), or a recommendation to a healthcare provider is made to alter the FXN replacement therapy regimen.

In some embodiments, when there is lack of a decrease (e.g., no difference) in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject as compared to the amount in the normal FXN expression profile, then an FXN replacement therapy is not administered to the FXN deficient subject, or a recommendation to a healthcare provider is made to not administer an FXN replacement therapy to the subject. In some embodiments, when there is a lack of a decrease (e.g., no difference) in the amount of any one or more of Timp, LRRK2, BASP1, CD44, Xpo6, SOD2, PSMB9, FTL, TXNIP, EGR1, CTSS, BTG2, PTGS2, LY96, Adgre1, PSMB8, C3ar1, TFRC, PLCL2, S100a4, Mapre1, CFH, and/or Tmem70 in the sample obtained from the FXN deficient subject as compared to the amount in the normal FXN expression profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is an increase in the amount of any one or more of Lgals3, Ube2v2, Sec61g, Zdhhc13, Yipf5, Tpp2, Ybx2 and/or Ptms in the sample obtained from the FXN deficient subject as compared to the amount in the normal FXN expression profile, then an FXN replacement therapy is administered to the FXN deficient subject, or a recommendation to a healthcare provider is made to administer an FXN replacement therapy to the FXN deficient subject. In some embodiments, when there is an increase in the amount of any one or more of Lgals3, Ube2v2, Sec61g, Zdhhc13, Yipf5, Tpp2, Ybx2 and/or Ptms in the sample obtained from the FXN deficient subject as compared to the amount in the normal FXN expression profile, and the FXN deficient subject is undergoing an FXN replacement therapy, then the FXN replacement therapy regimen is altered (e.g., the dose and or frequency of administration is increased), or a recommendation to a healthcare provider is made to alter the FXN replacement therapy regimen.

In some embodiments, when there is lack of an increase (e.g. no difference) in the amount of any one or more of Lgals3, Ube2v2, Sec61g, Zdhhc13, Yipf5, Tpp2, Ybx2 and/or Ptms in the sample obtained from the FXN deficient subject as compared to the amount in the normal FXN expression profile, then an FXN replacement therapy is not administered to the FXN deficient subject, or a recommendation is made to a healthcare provider to not administer an FXN replacement therapy. In some embodiments, when there is lack of an increase (e.g., no difference) in the amount of any one or more of Lgals3, Ube2v2, Sec61g, Zdhhc13, Yipf5, Tpp2, Ybx2 and/or Ptms in the sample obtained from the FXN deficient subject as compared to the amount in the normal FXN expression profile, and wherein the FXN deficient subject is undergoing FXN replacement therapy, then the FXN replacement therapy regimen is maintained, or a recommendation is made to a healthcare provider to maintain the FXN replacement therapy regimen.

In some embodiments, the methods of the present disclosure are carried out by the subject using a sample obtained from the same subject or as a point of care test, and results can be assessed by the subject or by a physician. In one aspect, the present invention constitutes an application of information obtainable by the methods of the invention in connection with analyzing, detecting, and/or measuring one or more of the FSGMs of the present invention, i.e., the FSGMs listed in Table 1 and/or Table 5, alone or in combination with one or more additional FSGMs listed in Tables 2, 3, 4 and/or 6. In one embodiment, the one or more FSGM comprises one or any combination of Timp1, LRRK2, BASP1, CD44, Xpo6, Lgals3, SOD2, PSMB9, FTL and TXNIP. In another embodiment, the one or more FSGM comprises one or more of Timp1, LRRK2, BASP1, CD44, Xpo6, Lgals3, SOD2, PSMB9, FTL and TXNIP, alone or in combination with one or more additional FSGMs in Tables 2, 3, 4 and/or 6, e.g., one or more of CTSS, BTG2, EGR1 and PTGS2.

For example, when executing the methods of the invention for detecting and/or measuring one or more protein FSGM of the present invention, i.e., one or more of the FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2, as described herein, one may contact a biological sample with a detection reagent, e.g., a monoclonal antibody, which selectively binds to the FSGM of interest, forming a protein-protein complex, which is then further detected either directly (if the antibody comprises a label) or indirectly (if a secondary detection reagent is used, e.g., a secondary antibody, which in turn is labeled). Thus, the method of the invention transforms the polypeptide FSGMs of the invention, i.e., one or more of the FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2, to a protein-protein complex that comprises either a detectable primary antibody or a primary and further secondary antibody. Forming such protein-protein complexes is required in order to identify the presence of the FSGM of interest and necessarily changes the physical characteristics and properties of the FSGM of interest as a result of conducting the methods of the invention.

The same principal applies when conducting the methods of the invention for detecting nucleic acids that correspond to one or more of the FSGMs of the invention, i.e., one or more of the FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2. In particular, when amplification methods are used, the process results in the formation of a new population of amplicons, i.e., molecules that are newly synthesized and which were not present in the original biological sample, thereby physically transforming the biological sample. Similarly, when hybridization probes are used to detect a target FSGM, a physical new species of molecules is in effect created by the hybridization of the probes (optionally comprising a label) to the target biomarker mRNA (or other nucleic acid), which is then detected. Such polynucleotide products are effectively newly created or formed as a consequence of carrying out the methods of the invention.

Methods for monitoring or evaluating the efficacy of FXN replacement therapy in a subject over time are also provided. In these methods the amount of one or more FSGM, i.e., one or more of the FSGMs listed in Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2, in a pair of samples (a first sample obtained from the subject at an earlier time point or prior to the treatment regimen and a second sample obtained from the subject at a later time point, e.g., at a later time point when the subject has undergone at least a portion of the treatment regimen) is assessed. It is understood that the methods of the invention include obtaining and analyzing more than two samples (e.g., 3, 4, 5, 6, 7, 8, 9, or more samples) at regular or irregular intervals for assessment of FSGM levels. Pairwise comparisons can be made between consecutive or non-consecutive subject samples. Trends of FSGM levels and rates of change of FSGM levels can be analyzed for any two or more consecutive or non-consecutive subject samples.

An exemplary method for detecting the presence or absence or change of expression level of an FSGM protein or a corresponding nucleic acid in a biological sample involves obtaining a biological sample from a subject and contacting the biological sample with a compound or an agent capable of detecting the polypeptide or nucleic acid (e.g., mRNA, genomic DNA, or cDNA). In some embodiments, the detection methods of the invention can thus be used to detect mRNA, protein, cDNA, or genomic DNA, for example, in the biological sample in vitro as well as in vivo.

Methods provided herein for detecting the presence, absence, change of expression level of an FSGM protein, e.g., one or more of the FSGMs listed in Table 1 and/or Table 5, alone or in combination with one or more additional FSGMs listed in Tables 2, 3, 4 and/or 6, or corresponding nucleic acid, in a biological sample include obtaining a biological sample from a subject that may or may not contain the FSGM protein or nucleic acid to be detected, contacting the sample with an FSGM-specific binding agent (i.e., one or more FSGM-specific binding agents) that is capable of forming a complex with the FSGM protein or nucleic acid to be detected, and contacting the sample with a detection reagent for detection of the FSGM—FSGM-specific binding agent complex, if formed. It is understood that the methods provided herein for detecting an expression level of an FSGM in a biological sample includes the steps to perform the assay. In certain embodiments of the detection methods, the level of the FSGM protein or nucleic acid in the sample is none or below the threshold for detection.

The methods include formation of either a transient or stable complex between the FSGM and the FSGM-specific binding agent. The methods require that the complex, if formed, be formed for sufficient time to allow a detection reagent to bind the complex and produce a detectable signal (e.g., fluorescent signal, a signal from a product of an enzymatic reaction, e.g., a peroxidase reaction, a phosphatase reaction, a beta-galactosidase reaction, or a polymerase reaction).

In certain embodiments, all FSGMs are detected using the same method. In certain embodiments, all FSGMs are detected using the same biological sample (e.g., same body fluid or tissue). In certain embodiments, different FSGMs are detected using various methods. In certain embodiments, FSGMs are detected in different biological samples. In some embodiments, a biological sample is a solid tissue sample, preferably a buccal sample, alternatively a skin biopsy sample, skin strip, hair follicle, muscle biopsy sample, or a body fluid sample such as blood (including any blood product, such as whole blood, plasma, serum, or specific types of cells of the blood), urine, saliva, or seminal fluid. In a preferred embodiment, the biological sample is a buccal sample.

FSGM levels can be detected based on the absolute expression level or a normalized or relative expression level. Detection of absolute FSGM levels may be preferable when monitoring the treatment of a subject or in determining if there is a change in the FXN status of a subject. For example, the expression level of one or more FSGMs can be monitored in a subject undergoing treatment with an FXN replacement therapy, e.g., at regular intervals, such as a monthly intervals. A modulation in the level of one or more FSGMs can be monitored over time to observe trends in changes of the FSGM levels. Expression levels of the FSGMs of the invention in the subject may be higher than the expression level of those FSGMs in a normal sample, but may be lower than the prior expression level, thus indicating a lack of efficacy of the FXN replacement therapy in the subject. Changes, or not, in FSGM levels may be more relevant to treatment decisions for the subject than FSGM levels present in the population. Rapid changes in FSGM levels in a subject may be indicative of an abnormal FXN levels, even if the FSGMs are within normal ranges for the population.

As an alternative to making determinations based on the absolute expression level of the FSGM, determinations may be based on the normalized expression level of the FSGM. Expression levels are normalized by correcting the absolute expression level of an FSGM by comparing its expression to the expression of a gene that is not an FSGM, e.g., a housekeeping gene that is constitutively expressed. Suitable genes for normalization include housekeeping genes such as the actin gene, or epithelial cell-specific genes. This normalization allows the comparison of the expression level in one sample, e.g., a sample from an FXN deficient subject, to another sample, e.g., a normal sample, or between samples from different sources.

The present disclosure describes a method for evaluating and/or monitoring effectiveness of treatment with FXN replacement therapy for a subject in need thereof, in which a sample from the subject is analyzed. As used herein, a sample may be a solid tissue sample, preferably a buccal sample, alternatively a skin biopsy sample, muscle biopsy sample, or a body fluid sample, such as a blood sample for example. Essentially, a sample of any tissue or body fluid that comprises cells in which FXN expression profile may be analyzed may be used in any of the methods disclosed herein. Alternatively, exosomes may be harvested in order to be tested for FSGM transcripts. In a preferred embodiment, the biological sample is a buccal sample.

In some embodiments, the baseline FXN(−) expression profile may be determined using samples from human subjects with FRDA who are FXN deficient, while normal FXN expression profile may be determined using healthy subjects who are not FXN deficient. Treatment of human subjects with FRDA with FXN-replacement therapy, such as an FXN fusion protein, results in regulation of FSGMs in a dose-dependent manner, such that their FXN expression profiles became similar to normal FXN expression profile after treatment.

Any one of the FXN expression profiles described herein may be part of one or more algorithms which may be used to analyze the FXN expression profile of a sample and determine whether the sample represents a sample of a normal subject, a sample from a subject prior to FXN replacement therapy or a sample from a subject after FXN replacement therapy. The one or more algorithms may be used to analyze a sample from a subject treated with an FXN-replacement therapy and determine whether the subject has reacted effectively to the treatment, and therefore expresses a profile characteristic of an FXN replacement expression profile or not. Thus an algorithm for analyzing the expression profile of a sample may use any one of a baseline FXN(−) expression profile, an FXN replacement expression profile, or a normal FXN expression profile, or a combination of profiles. In such methods, a sample having FXN signature expression patterns consistent with baseline FXN(−) expression profile represents lack of effectiveness of FXN replacement therapy, and a sample having FXN expression profile consistent with FXN replacement expression profile and/or normal FXN expression profile represents effectiveness of FXN replacement therapy. In an embodiment, a classifier may be applied to FXN expression profiles obtained from subject samples in order to obtain information about the samples, for example to characterize the status of the FXN expression profile, or to define whether the subject was administered FXN replacement therapy or not. Alternatively or additionally, a classifier may be applied for evaluating whether the FXN expression profile of the subject sample reached a certain threshold necessary for FXN replacement treatment to be considered effective.

Also provided in the disclosure is a method of treatment of a subject suffering from a mitochondrial disease having FXN deficiency, the method comprising determining an FXN expression profile in a sample from the subject, and comparing the FXN expression profile obtained from the sample with at least one of a normal FXN expression profile, a baseline FXN(−) expression profile, or an FXN replacement expression profile. The sample may be further classified as having a normal FXN, a baseline FXN(−) or an FXN replacement profile. Using the results of the comparison of the sample FXN profile with the FXN profiles described herein, a therapy regime using FXN replacement therapy may be initiated, paused or ceased. Alternatively, an FXN replacement therapy dosage regime may be modified, e.g., increased or decreased. In one embodiment, the method further comprises obtaining or providing a sample from a subject, e.g., a subject suffering from FXN deficiency, such as a subject with FRDA.

In certain embodiments of the methods provided herein, a change (an increase or decrease) in the level of one or more FSGMs selected from Table 1 and/or Table 5, alone or in combination with one or more additional FSGMs listed in Tables 2, 3, 4 and/or 6 (e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2), in the biological sample as compared to the level of the one or more FSGMs in a control sample, e.g., a sample from a subject deficient for FXN, is an indication that the FXN replacement therapy is effective.

Specifically, in some embodiments, an increase in the level of Timp1 in a sample obtained from an FXN deficient subject following administration of FXN replacement therapy, as compared to the level of Timp1 in a sample obtained from the subject prior to administration of the FXN replacement therapy, is an indication that the FXN replacement therapy is effective.

In some embodiments, an increase in the level of LRRK2 in a sample obtained from an FXN deficient subject following administration of FXN replacement therapy, as compared to the level of LRKK2 in a sample obtained from the subject prior to administration of the FXN replacement therapy, is an indication that the FXN replacement therapy is effective.

In some embodiments, an increase in the level of BASP1 in a sample obtained from an FXN deficient subject following administration of FXN replacement therapy, as compared to the level of BASP1 in a sample obtained from the subject prior to administration of the FXN replacement therapy, is an indication that the FXN replacement therapy is effective.

In some embodiments, an increase in the level of CD44 in a sample obtained from an FXN deficient subject following administration of FXN replacement therapy, as compared to the level of CD44 in a sample obtained from the subject prior to administration of the FXN replacement therapy, is an indication that the FXN replacement therapy is effective.

In some embodiments, an increase in the level of Xpo6 in a sample obtained from an FXN deficient subject following administration of FXN replacement therapy, as compared to the level of Xpo6 in a sample obtained from the subject prior to administration of the FXN replacement therapy, is an indication that the FXN replacement therapy is effective.

In some embodiments, an decrease in the level of Lgals3 in a sample obtained from an FXN deficient subject following administration of FXN replacement therapy, as compared to the level of Lgals3 in a sample obtained from the subject prior to administration of the FXN replacement therapy, is an indication that the FXN replacement therapy is effective.

In some embodiments, an increase in the level of SOD2 in a sample obtained from an FXN deficient subject following administration of FXN replacement therapy, as compared to the level of SOD2 in a sample obtained from the subject prior to administration of the FXN replacement therapy, is an indication that the FXN replacement therapy is effective.

In some embodiments, an increase in the level of PSMB9 in a sample obtained from an FXN deficient subject following administration of FXN replacement therapy, as compared to the level of PSMB9 in a sample obtained from the subject prior to administration of the FXN replacement therapy, is an indication that the FXN replacement therapy is effective.

In some embodiments, an increase in the level of FTL in a sample obtained from an FXN deficient subject following administration of FXN replacement therapy, as compared to the level of PSMB9 in a sample obtained from the subject prior to administration of the FXN replacement therapy, is an indication that the FXN replacement therapy is effective.

In some embodiments, an increase in the level of TXNIP in a sample obtained from an FXN deficient subject following administration of FXN replacement therapy, as compared to the level of PSMB9 in a sample obtained from the subject prior to administration of the FXN replacement therapy, is an indication that the FXN replacement therapy is effective.

In some embodiments, an increase in the level of EGR1 in a sample obtained from an FXN deficient subject following administration of FXN replacement therapy, as compared to the level of EGR1 in a sample obtained from the subject prior to administration of the FXN replacement therapy, is an indication that the FXN replacement therapy is effective.

In some embodiments, an increase in the level of CTSS in a sample obtained from an FXN deficient subject following administration of FXN replacement therapy, as compared to the level of CTSS in a sample obtained from the subject prior to administration of the FXN replacement therapy, is an indication that the FXN replacement therapy is effective.

In some embodiments, an increase in the level of BTG2 in a sample obtained from an FXN deficient subject following administration of FXN replacement therapy, as compared to the level of BTG2 in a sample obtained from the subject prior to administration of the FXN replacement therapy, is an indication that the FXN replacement therapy is effective.

In some embodiments, an increase in the level of PTGS2 in a sample obtained from an FXN deficient subject following administration of FXN replacement therapy, as compared to the level of PTGS2 in a sample obtained from the subject prior to administration of the FXN replacement therapy, is an indication that the FXN replacement therapy is effective.

In some embodiments, an increase in the level of LY96 in a sample obtained from an FXN deficient subject following administration of FXN replacement therapy, as compared to the level of LY96 in a sample obtained from the subject prior to administration of the FXN replacement therapy, is an indication that the FXN replacement therapy is effective.

In some embodiments, an increase in the level of Adgre1 in a sample obtained from an FXN deficient subject following administration of FXN replacement therapy, as compared to the level of Adgre1 in a sample obtained from the subject prior to administration of the FXN replacement therapy, is an indication that the FXN replacement therapy is effective.

In some embodiments, an increase in the level of PSMB8 in a sample obtained from an FXN deficient subject following administration of FXN replacement therapy, as compared to the level of PSMB8 in a sample obtained from the subject prior to administration of the FXN replacement therapy, is an indication that the FXN replacement therapy is effective.

In some embodiments, an increase in the level of C3ar1 in a sample obtained from an FXN deficient subject following administration of FXN replacement therapy, as compared to the level of C3ar1 in a sample obtained from the subject prior to administration of the FXN replacement therapy, is an indication that the FXN replacement therapy is effective.

In some embodiments, an increase in the level of TFRC in a sample obtained from an FXN deficient subject following administration of FXN replacement therapy, as compared to the level of TFRC in a sample obtained from the subject prior to administration of the FXN replacement therapy, is an indication that the FXN replacement therapy is effective.

In some embodiments, an increase in the level of PLCL2 in a sample obtained from an FXN deficient subject following administration of FXN replacement therapy, as compared to the level of PLCL2 in a sample obtained from the subject prior to administration of the FXN replacement therapy, is an indication that the FXN replacement therapy is effective.

In some embodiments, an increase in the level of S100a4 in a sample obtained from an FXN deficient subject following administration of FXN replacement therapy, as compared to the level of S100a4 in a sample obtained from the subject prior to administration of the FXN replacement therapy, is an indication that the FXN replacement therapy is effective.

In some embodiments, an increase in the level of Mapre1 in a sample obtained from an FXN deficient subject following administration of FXN replacement therapy, as compared to the level of Mapre1 in a sample obtained from the subject prior to administration of the FXN replacement therapy, is an indication that the FXN replacement therapy is effective.

In some embodiments, an increase in the level of CFH in a sample obtained from an FXN deficient subject following administration of FXN replacement therapy, as compared to the level of CFH in a sample obtained from the subject prior to administration of the FXN replacement therapy, is an indication that the FXN replacement therapy is effective.

In some embodiments, an increase in the level of Tmem70 in a sample obtained from an FXN deficient subject following administration of FXN replacement therapy, as compared to the level of Tmem70 in a sample obtained from the subject prior to administration of the FXN replacement therapy, is an indication that the FXN replacement therapy is effective.

In some embodiments, a decrease in the level of Ube2v2 in a sample obtained from an FXN deficient subject following administration of FXN replacement therapy, as compared to the level of Ube2v2 in a sample obtained from the subject prior to administration of the FXN replacement therapy, is an indication that the FXN replacement therapy is effective.

In some embodiments, a decrease in the level of Sec61g in a sample obtained from an FXN deficient subject following administration of FXN replacement therapy, as compared to the level of Sec61g in a sample obtained from the subject prior to administration of the FXN replacement therapy, is an indication that the FXN replacement therapy is effective.

In some embodiments, a decrease in the level of Zdhhc13 in a sample obtained from an FXN deficient subject following administration of FXN replacement therapy, as compared to the level of Zdhhc13 in a sample obtained from the subject prior to administration of the FXN replacement therapy, is an indication that the FXN replacement therapy is effective.

In some embodiments, a decrease in the level of Yipf5 in a sample obtained from an FXN deficient subject following administration of FXN replacement therapy, as compared to the level of Yipf5 in a sample obtained from the subject prior to administration of the FXN replacement therapy, is an indication that the FXN replacement therapy is effective.

In some embodiments, a decrease in the level of Tpp2 in a sample obtained from an FXN deficient subject following administration of FXN replacement therapy, as compared to the level of Tpp2 in a sample obtained from the subject prior to administration of the FXN replacement therapy, is an indication that the FXN replacement therapy is effective.

In some embodiments, a decrease in the level of Ybx2 in a sample obtained from an FXN deficient subject following administration of FXN replacement therapy, as compared to the level of Ybx2 in a sample obtained from the subject prior to administration of the FXN replacement therapy, is an indication that the FXN replacement therapy is effective.

In some embodiments, a decrease in the level of Ptms in a sample obtained from an FXN deficient subject following administration of FXN replacement therapy, as compared to the level of Ptms in a sample obtained from the subject prior to administration of the FXN replacement therapy, is an indication that the FXN replacement therapy is effective.

In certain embodiments of the methods provided herein, no change (no increase or decrease) in the detected expression level of one or more FSGMs selected from Table 1 and/or Table 5, alone or in combination with one or more additional FSGMs listed in Tables 2, 3, 4 and/or 6 (e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2), in the biological sample as compared to the expression level of the one or more FSGMs in a sample from a subject deficient for FXN, is an indication that the FXN replacement therapy is ineffective, e.g., at the current dose, and should be modified.

In certain embodiments, the methods may also include monitoring a subject being administered FXN replacement therapy. In some embodiments, no increase or decrease in the detected expression level of one or more FSGMs selected from Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2, in a second sample obtained from a subject after FXN replacement therapy is administered to the subject, as compared to the level of the one or more FSGMs in a first sample obtained from the subject before FXN replacement therapy is administered to the subject, is an indication that the FXN replacement therapy is not efficacious and/or the subject is not responsive FXN replacement therapy. The method may further include the step of adjusting the FXN replacement therapy to a higher dose.

In other embodiments, an increased or decreased expression level of one or more FSGMs selected from Table 1 and/or Table 5, alone or in combination with one or more additional FSGMs listed in Tables 2, 3, 4 and/or 6, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2, in a second sample obtained from a subject after FXN replacement therapy is administered to the subject, as compared to the expression level of the one or more FSGMs, e.g., one or more of Timp1, LRRK2, BASP1, CD44, Xpo6, Lgals3, SOD2, PSMB9, FTL and TXNIP, alone or in combination with one or more of CTSS, BTG2, EGR1 and PTGS2, in a first sample obtained from the subject before FXN replacement therapy is administered, is an indication that the FXN replacement therapy is efficacious and/or the subject is responsive to the FXN replacement therapy. The method may further include the step of adjusting the FXN replacement therapy to a lower dose or ceasing the therapy.

In certain embodiments, the level of the FSGM is increased following treatment of a subject with an FXN replacement, e.g., a subject deficient in FXN. In some embodiments, the FSGM is selected from the group consisting of Timp1, LRRK2, BASP1, CD44, Xpo6, SOD2, PSMB9, FTL and TXNIP.

In some embodiments, the level of Timp1 is increased following treatment of an FXN deficient subject with FXN replacement therapy. In some embodiments, the level of LRRK2 is increased following treatment of an FXN deficient subject with FXN replacement therapy. In some embodiments, the level of BASP1 is increased following treatment of an FXN deficient subject with FXN replacement therapy. In some embodiments, the level of CD44 is increased following treatment of an FXN deficient subject with FXN replacement therapy. In some embodiments, the level of Xpo6 is increased following treatment of an FXN deficient subject with FXN replacement therapy. In some embodiments, the level of SOD2 is increased following treatment of an FXN deficient subject with FXN replacement therapy. In some embodiments, the level of PSMB9 is increased following treatment of an FXN deficient subject with FXN replacement therapy. In some embodiments, the level of FTL is increased following treatment of an FXN deficient subject with FXN replacement therapy. In some embodiments, the level of TXNIP is increased following treatment of an FXN deficient subject with FXN replacement therapy. In some embodiments, the level of EGR1 is increased following treatment of an FXN deficient subject with FXN replacement therapy. In some embodiments, the level of CTSS is increased following treatment of an FXN deficient subject with FXN replacement therapy. In some embodiments, the level of BTG2 is increased following treatment of an FXN deficient subject with FXN replacement therapy. In some embodiments, the level of PTS2 is increased following treatment of an FXN deficient subject with FXN replacement therapy.

In other embodiments, the level of the FSGM is decreased following treatment of a subject with an FXN replacement, e.g., a subject deficient in FXN. In some embodiments, the FSGM is Lgals3.

In other embodiments, the present invention also involves the analysis and consideration of any clinical and/or subject-related health data, for example, data obtained from an Electronic Medical Record (e.g., collection of electronic health information about individual subjects or populations relating to various types of data, such as, demographics, medical history, medication and allergies, immunization status, laboratory test results, radiology images, vital signs, personal statistics like age and weight, and billing information).

In certain embodiments the methods provided herein further comprise obtaining a biological sample from a subject suspected of having a mitochondrial disease, e.g., 1-RDA.

In certain embodiments the methods provided herein further comprise selecting a treatment regimen for the subject based on the level of the one or more FSGMs selected from Table 1 and/or Table 5, alone or in combination with one or more additional FSGMs listed in Tables 2, 3, 4 and/or 6. In certain embodiments, the treatment method is started, change, revised, or maintained based on the results from the methods of the invention, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the methods.

In certain embodiments of the diagnostic and monitoring methods provided herein, the method further comprises isolating a component (e.g., protein or mRNA) of the biological sample. In certain embodiments of the diagnostic and monitoring methods provided herein, the method further comprises labeling a component (e.g., protein or mRNA) of the biological sample. In certain embodiments of the diagnostic and monitoring methods provided herein, the method further comprises amplifying a component (e.g., mRNA) of a biological sample.

In certain embodiments of the methods provided herein, the method comprises forming a complex between a probe and a component of a biological sample. In certain embodiments, forming a complex with a probe comprises forming a complex with at least one non-naturally occurring reagent. In certain embodiments of the methods provided herein, the method comprises processing the biological sample. In certain embodiments of the methods provided herein, the method of detecting a level of at least two FSGMs comprises a panel of FSGMs. In certain embodiments of the methods provided herein, the method of detecting a level comprises attaching the FSGM to be detected to a solid surface.

In another aspect, the present invention provides for the identification of a “diagnostic signature” or “diagnostic expression profile” based on the levels of the FSGMs of the invention in a biological sample, that correlates with FXN in the sample. The “levels of the FSGMs” can refer to the protein level of an FSGM in a biological sample. The “levels of the FSGMs” can also refer to the expression level of the genes corresponding to the proteins, e.g., by measuring the expression levels of the corresponding FSGM mRNAs. The collection or totality of levels of FSGMs provide a diagnostic signature that correlates with the level of FXN.

### I. Kits/Panels

The invention also provides compositions and kits comprising detection reagents for determining FXN expression profiles of FSGMs, as described herein. The compositions and kits are useful for carrying out the methods described herein, e.g., for evaluating and monitoring effectiveness of FXN replacement therapy. In some embodiments, the kits of the disclosure may be used by a subject for self-evaluation or may be carried out by a subject for evaluation by a physician, or as point of care kits.

These kits may include one or more of the following: a reagent that specifically binds to an FSGM of the invention, and a set of instructions for measuring the level of the FSGM. In one embodiment, the FSGM comprises any one of the FSGMs listed in Table 1 and/or Table 5 (e.g., any one or more of Timp1, LRRK2, BASP1, CD44, Xpo6, Lgals3, SOD2, PSMB9, FTL and TXNIP), alone or in combination with one or more FSGMs listed in Tables 2, 3, 4 and/or 6 (e.g., CTSS, BTG2, EGR1 and PTGS2).

The invention also encompasses kits for detecting the presence of an FSGM protein or nucleic acid in a biological sample. Such kits can be used to determine FXN expression profiles of FSGMs in a sample from a subject, and to evaluate and/or monitor effectiveness of FXN replacement therapy. For example, the kit can comprise a labeled compound or agent capable of detecting an FSGM protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for use of the kit for practicing any of the methods provided herein or interpreting the results obtained using the kit based on the teachings provided herein. The kits can also include reagents for detection of a control protein in the sample, e.g., actin for tissue samples, albumin in blood or blood derived samples for normalization of the amount of the FSGM present in the sample. The kit can also include the purified FSGM that is to be detected in samples from subjects, for use as a control or for quantitation of the assay performed with the kit. In some embodiments, a biological sample which is evaluated by a kit or panel of the disclosure is a solid tissue sample, preferably a buccal sample, alternatively, a skin biopsy sample, skin strip, hair follicle, muscle biopsy sample, or a body fluid sample such as blood (including any blood product, such as whole blood, plasma, serum, or specific types of cells of the blood), urine, saliva, or seminal fluid.

In some embodiments, kits include a panel of reagents for use in methods of the present disclosure, e.g., to determine an FXN expression profile for FXGMs in a sample from an FXN deficient subject, or to evaluate and/or monitor effectiveness of an FXN replacement therapy, the panel comprising at least two detection reagents, wherein each detection reagent is specific for one FSGM, wherein said FSGMs are selected from the FSGMs provided herein. In one embodiment, at least one of the FSGMs comprises a protein encoded by any one of FSGMs listed in Table 1 and/or Table 5 (e.g., one or more of Timp1, LRRK2, BASP1, CD44, Xpo6, Lgals3, SOD2, PSMB9, FTL and TXNIP), alone or in combination with one or more FSGMs listed in Tables 2, 3, 4 and/or 6 (e.g., one or more of CTSS, BTG2, EGR1 and PTGS2).

For antibody-based kits, the kit can comprise, for example: (1) a first antibody (e.g., attached to a solid support) which binds to a first FSGM protein; and, optionally, (2) a second, different antibody which binds to either the first FSGM protein or the first antibody and is conjugated to a detectable label. In certain embodiments, the kit includes (1) a second antibody (e.g., attached to a solid support) which binds to a second FSGM protein; and, optionally, (2) a third, different antibody which binds to either the second FSGM protein or the second antibody and is conjugated to a detectable label. The first and second FSGM proteins are different. In an embodiment, the first and second FSGMs are FSGMs of the invention, e.g., one or more of the FSGMs selected from Table 1 and/or Table 5. In certain embodiments, the kit comprises a third antibody which binds to a third FSGM protein which is different from the first and second FSGM proteins, and a fourth different antibody that binds to either the third FSGM protein or the antibody that binds the third FSGM protein wherein the third FSGM protein is different from the first and second FSGM proteins.

For oligonucleotide-based kits, the kit can comprise, for example: (1) an oligonucleotide, e.g., a detectably labeled oligonucleotide, which hybridizes to a nucleic acid sequence encoding an FSGM protein or (2) a pair of primers useful for amplifying an FSGM nucleic acid molecule. In certain embodiments, the kit can further include, for example: (1) an oligonucleotide, e.g., a second detectably labeled oligonucleotide, which hybridizes to a nucleic acid sequence encoding a second FSGM protein or (2) a pair of primers useful for amplifying the second FSGM nucleic acid molecule. The first and second FSGMs are different. In an embodiment, the first and second FSGMs are FSGMs of the invention, e.g., one or more of the FSGMs selected from Table 1 and/or Table 5. In certain embodiments, the kit can further include, for example: (1) an oligonucleotide, e.g., a third detectably labeled oligonucleotide, which hybridizes to a nucleic acid sequence encoding a third FSGM protein or (2) a pair of primers useful for amplifying the third FSGM nucleic acid molecule wherein the third FSGM is different from the first and second FSGMs. In certain embodiments, the kit includes a third primer specific for each nucleic acid FSGM to allow for detection using quantitative PCR methods.

For chromatography methods, the kit can include FSGMs, including labeled FSGMs, to permit detection and identification of one or more FSGMs of the invention, e.g., one or more of the FSGMs listed in Table 1 and/or Table 5, alone or in combination with one or more additional FSGMs listed in Tables 2, 3, 4 and/or 6, by chromatography. In certain embodiments, kits for chromatography methods include compounds for derivatization of one or more FSGMs of the invention. In certain embodiments, kits for chromatography methods include columns for resolving the FSGMs of the method.

Reagents specific for detection of an FSGM of the invention, e.g., one or more of the FSGMs listed in Table 1 and/or Table 5, alone or in combination with one or more additional FSGMs listed in Tables 2, 3, 4 and/or 6, allow for detection and quantitation of the FSGM in a complex mixture, e.g., cell or tissue sample. In certain embodiments, the reagents are species specific. In certain embodiments, the reagents are not species specific. In certain embodiments, the reagents are isoform specific. In certain embodiments, the reagents are not isoform specific.

In certain embodiments, the kits useful in methods for evaluation and/or monitoring of the effectiveness of FXN replacement therapy comprise at least one reagent specific for the detection of the level of one or more of the FSGMs listed in Table 1 and/or Table 5. In some embodiments, the kits further comprise at least one reagent specific for the detection of the level of one or more additional FSGMs listed in Tables 2, 3, 4 and/or 6. In certain embodiments, the kits further comprise instructions for the detection, evaluation and/or monitoring of the effectiveness of FXN replacement therapy based on the level of at least one FSGM listed in Table 1 and/or Table 5, alone or in combination with one or more additional FSGMs listed in Tables 2, 3, 4 and/or 6.

In some embodiments, the present disclosure provides kits comprising at least one reagent specific for the detection of the level of at least one FSGM listed in Table 1. In some embodiments, the present disclosure provides kits comprising at least one reagent specific for the detection of the level of at least one FSGM listed in Table 5. In one embodiment, the FSGMs comprise one or more of Timp1, LRRK2, BASP1, CD44, Xpo6, Lgals3, SOD2, PSMB9, FTL and TXNIP. In one embodiment, the FSGMs comprises Timp1, LRRK2, BASP1, CD44, Xpo6, Lgals3, SOD2, PSMB9, FTL and TXNIP. In one embodiment, the FSGMs comprise Xpo6, SOD2, CTSS, EGR1, BTG2, PTSG2 and LRKK2. In one embodiment, the reagent detects a protein. In another embodiment, the reagent detects an mRNA.

In certain embodiments, the kits can also comprise, e.g., a buffering agents, a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate, with known levels of target FSGMs. Each component of the kit can be enclosed within an individual container and all of the various containers can be within a single package, along with instructions for interpreting the results of the assays performed using the kit. The kits of the invention may optionally comprise additional components useful for performing the methods of the invention.

The present disclosure further provides a panel of reagents, which are useful in the methods provided herein. The panel of reagents comprises at least two detection reagents, wherein each detection reagent is specific for the detection in a subject sample of at least one FSGM in a set of FSGMs, where the set of FSGMs comprises two or more FSGMs disclosed herein. In some embodiments, the panel of reagents further includes at least one control reagent. In certain embodiments, the set of FSGMs comprises at least two or more FSGMs, wherein at least one of the FSGMs is selected from Table 1 and/or Table 5. In one embodiment, the two or more FSGMs comprises a protein encoded by FSGMs listed in Table 1 and/or Table 5, such as two or more of Timp1, LRRK2, BASP1, CD44, Xpo6, Lgals3, SOD2, PSMB9, FTL and TXNIP. In some embodiments, the two or more FSGM are selected from the group consisting of Timp1, LRRK2, BASP1, CD44, Xpo6, SOD2, PSMB9, FTL and TXNIP. In some embodiments, the two or more FSGMs comprise Xpo6, SOD2, CTSS, EGR1, BTG2, PTSG2 and LRKK2. In certain embodiments, the the two or more FSGMs comprise Lgals3.

In certain embodiments, the control reagent in the panel is used to detect the FSGM that is to be detected in the biological sample from a subject, wherein the panel is provided with a control sample containing the FSGM for use as a positive control and optionally to quantitate the amount of FSGM present in the biological sample. In some embodiments, the panel can be provided with reagents for detection of a control protein, e.g., actin for tissue samples, albumin in blood or blood derived samples for normalization of the amount of the FSGM present in the sample. In some embodiments, the panel can be provided with a purified FSGM for detection for use as a control or for quantitation of the assay performed with the panel.

In some embodiments, the panel comprises reagents for detection of one or more FSGMs with an increased level when compared to a control following treatment of a subject with FXN replacement, e.g., a subject deficient in FXN, and/or one or more FSGMs with a decreased level when compared to a control or following treatment of a subject with FXN replacement, e.g., a subject deficient in FXN.

In a preferred embodiment, the panel includes reagents for detection of two or more FSGMs of the invention (e.g., 2, 3, 4, 5, 6, 7, 8, 9 or up to all of the FSGMs listed in Table 1 and/or Table 5, alone or in combination with one or more of additional FSGMs listed in Tables 2, 3, 4 and/or 6), preferably in conjunction with a control reagent. In some embodiments, the panel includes reagents for detection of one or more of Timp1, LRRK2, BASP1, CD44, Xpo6, Lgals3, SOD2, PSMB9, FTL and TXNIP. In some embodiments, the panel includes reagents for detection of two or more of Timp1, LRRK2, BASP1, CD44, Xpo6, Lgals3, SOD2, PSMB9, FTL and TXNIP; three or more of Timp1, LRRK2, BASP1, CD44, Xpo6, Lgals3, SOD2, PSMB9, FTL and TXNIP; four or more of Timp1, LRRK2, BASP1, CD44, Xpo6, Lgals3, SOD2, PSMB9, FTL and TXNIP; five or more of Timp1, LRRK2, BASP1, CD44, Xpo6, Lgals3, SOD2, PSMB9, FTL and TXNIP; six or more of Timp1, LRRK2, BASP1, CD44, Xpo6, Lgals3, SOD2, PSMB9, FTL and TXNIP; seven or more of Timp1, LRRK2, BASP1, CD44, Xpo6, Lgals3, SOD2, PSMB9, FTL and TXNIP; eight or more of Timp1, LRRK2, BASP1, CD44, Xpo6, Lgals3, SOD2, PSMB9, FTL and TXNIP; nine or more of Timp1, LRRK2, BASP1, CD44, Xpo6, Lgals3, SOD2, PSMB9, FTL and TXNIP; or all of Timp1, LRRK2, BASP1, CD44, Xpo6, Lgals3, SOD2, PSMB9, FTL and TXNIP.

In some embodiments, the panel includes reagents for detection of one or more additional FSGMs listed in Tables 2, 3, 4 and/or 6. In some embodiments, the panel includes reagents for detection of one or more of CTSS, BTG2, EGR1 and PTGS2; two or more of CTSS, BTG2, EGR1 and PTGS2; three or more of CTSS, BTG2, EGR1 and PTGS2; or all of BTG2, EGR1, and PTGS2.

In the panel, each FSGM is detected by a reagent specific for that FSGM. In certain embodiments, the panel includes replicate wells, spots, or portions to allow for analysis of various dilutions (e.g., serial dilutions) of biological samples and control samples. In a preferred embodiment, the panel allows for quantitative detection of one or more FSGMs of the invention. In certain embodiments, the panel is a protein chip for detection of one or more FSGMs. In certain embodiments, the panel is an ELISA plate for detection of one or more FSGMs. In certain embodiments, the panel is a plate for quantitative PCR for detection of one or more FSGMs.

In certain embodiments, the panel of detection reagents is provided on a single device including a detection reagent for one or more FSGMs of the invention and at least one control sample. In certain embodiments, the panel of detection reagents is provided on a single device including a detection reagent for two or more FSGMs of the invention and at least one control sample. In certain embodiments, multiple panels for the detection of different FSGMs of the invention are provided with at least one uniform control sample to facilitate comparison of results between panels.

### EXAMPLES

### Example 1. Identification of FSGMs in Human Subjects

### FXN Fusion Protein and FXN Replacement Therapy

The FXN replacement therapy used in this example and in Examples 2-5 involved administration of an exemplary FXN fusion protein. The exemplary FXN fusion protein is a fusion protein comprising TAT-cpp and human FXN (hFXN) linked through a linker at the N-terminus of hFXN. The hFXN in the exemplary FXN fusion protein is the full-length 210 aa frataxin long precursor form, which contains an 80 aa mitochondrial targeting sequence (MTS) at the N-terminus. The full-length hFXN protein (amino acids 1-210) has the amino acid sequence of SEQ ID NO: 1.

| | |
|---|---|
| SEQ ID NO: 1 | MWTLGRRAVAGLLASPSPAQAQTLTRVP |
| Full-length | RPAELAPLCGRRGLRTDDETTYERLAEE |
| hFXN | TLDSLAEFFEDLADKPYTFEDYDVSFGS |
| hFXN₁₋₂₁₀ | GVLTVKLGGDLGTYVINKQTPNKQIWLS |
| | SPSSGPKRYDWTGKNWVYSHDGVSLHEL |
| | LAAELTKALKTKLDLSSLAYSGKDAIDA |
| | TCTPRRASSNQRGLNQIWNVKKQSVYLM |
| | NLRKSGTLGHPGSL |

As the hFXN protein is imported into the mitochondrial matrix, it is cleaved at amino acid 81, resulting in the mature form of FXN, having 130 aa and a predicted molecular weight of 14.2 kDa (SEQ ID NO: 2).

| | |
|---|---|
| SEQ ID NO: 2 | SGTLGHPGSLDETTYERLAEETLDSLAEFFEDLA |
| Mature hFXN | DKPYTFEDYDVSFGSGVLTVKLGGDLGTYVINKQ |
| hFXN81-210 | TPNKQIWLSSPSSGPKRYDWTGKNWVYSHDGVSL |
| | HELLAAELTKALKTKLDLSSLAYSGKDA |

The full-length hFXN (SEQ ID NO: 1) comprises mature hFXN (SEQ ID NO: 2) and a mitochondrial targeting sequence (MTS) having the amino acid sequence

(SEQ ID NO: 3)
RGLNQIWNVKKQSVYLMNLRK.

The exemplary FXN fusion protein also includes the HIV-TAT peptide YGRKKRRQRRR (SEQ ID NO: 4) linked via a linker to the N-terminus of the full-length hFXN protein. The mechanism of action of the fusion protein relies on the cell-penetrating ability of the HIV-TAT peptide to deliver the fusion protein into cells and the subsequent processing into mature hFXN after translocation into the mitochondria. The exemplary FXN fusion protein used in this Example is described in US 2021/0047378, the entire contents of which are hereby incorporated herein by reference, and has the following amino acid sequence (224 amino acids):

(SEQ ID NO: 12)
MYGRKKRRQRRRGGMWTLGRRAVAGLLASPSPAQAQTLTRV
PRPAELAPLCGRRGLRTDIDATCTPRRASSNORGLNQIWNV
KKQSVYLMNLRKSGTLGHPGSLDETTYERLAEETLDSLAEF
FEDLADKPYTFEDYDVSFGSGVLTVKLGGDLGTYVINKQTP
NKQIWLSSPSSGPKRYDWTGKNWVYSHDGVSLHELLAAELT
KALKTKLDLSSLAYSGKDA.

Dosing with the Exemplary FXN Fusion Protein and Sample Collection

Samples used to identify FSGMs were collected from healthy volunteers and from FRDA subjects before and after administration of placebo or subcutaneous doses of 25 mg, 50 mg and 100 mg of the exemplary FXN fusion protein as a part of Multiple Ascenting Dose (MAD) study. Specifically, the dosing study involved 3 Cohorts: Cohort 1, Cohort 2 and Cohort 3. In Cohort 1, 2 FRDA subjects were dosed with placebo and 6 FRDA subjects were dosed subcutaneously with 25 mg of the exemplary FXN fusion protein on days 1, 2, 3, 4, 7, 10 and 13. In Cohort 2, 2 FRDA subjects were dosed with placebo and 7 FRDA subjects were dosed subcutaneously with 50 mg of the exemplary FXN fusion protein on days 1, 2, 3, 4, 5, 6, 7, 9, 11 and 13. In Cohort 3, 3 FRDA subjects were dosed with placebo and 3 FRDA subjects were dosed subcutaneously with 100 mg of the exemplary FXN fusion protein on days 1, 2, 3, 4, 5, 6, 7, 8, 9, 10, 11, 12 and 13.

Buccal samples were collected from all patients dosed with the exemplary FXN fusion protein at day −2 (prior to dosing), day 1, day 2, day 15, day 22 and day 43 following the first dose. Buccal samples were also collected from 10 healthy volunteers. The buccal sample collection for gene expression analysis was done using DNA/RNA Shield Collection Tube w/Swab (ZymoResearch, R1107) by swabbing the inside of each individual's cheek with either one swab for 4 minutes or two swabs for 1 minute each. Swabs were then returned to the collection tubes containing DNA/RNA Shield, and frozen at −80° C.

### RNA Extraction

Frozen buccal cell swabs were thawed and treated with proteinase K for 45 minutes at room temperature. Cells were then scraped off of the swab into the collection tube containing DNA/RNA shield and transferred to a Chemagic RNA extraction plate (Perkin Elmer, CMG-555-4). RNA was extracted using a Chemagic 360 automated nucleic acid isolation instrument (Perkin Elmer, 2024-0020) and its accompanying RNA extraction kit (Perkin Elmer, CMG-1084), according to the manufacture's instructions. Extracted RNA was quantified using a Nanodrop Lite spectrophotometer (Thermo Scientific, ND-LITE).

### Gene Expression Measurement

Gene expression was measured using Nanostring. Up to 250 ng of RNA was hybridized to custom nCounter Gene Expression Codesets (NanoString Technologies) by incubating with Reporter Probes and Capture Probes (Nanostring Technologies) for 18 hours at 65° C. in a thermocycler (Eppendorf 633000021), and then frozen at −80° C. The hybridized RNA/Codeset was thawed, then applied to a Nanostring imaging cartridge using an nCounter Prep Station (Nanostring Technologies, model 5s) according to the manufacturer's instructions. The cartridge was imaged using an nCounter Digital Analyzer (Nanostring Technologies, model 5s) according to the manufacturer's instructions.

### Data Analysis

Gene expression data was analyzed using nSolver 4.0 (Nanostring Technologies) and the nCounter Advanced Analysis software (version 2.0.134, Nanostring Technologies). More specifically, samples were normalized using housekeeping genes: ACTB, ATPL6V0C, BAD, CAMK2G, Ck5rap3, CHD4, Cldn12, ESAM, ITPR3, MAP2K2, SPAST, Vps28. Gene expression was compared between 1) healthy subjects and subjects with FRDA who were not treated with FXN replacement therapy (pre-dose), and 2) subjects with FRDA pre-dose and after treatment with FXN replacement therapy.

### Identification of FSGMs Based on Comparisons Between Healthy Subjects and FRDA Subjects

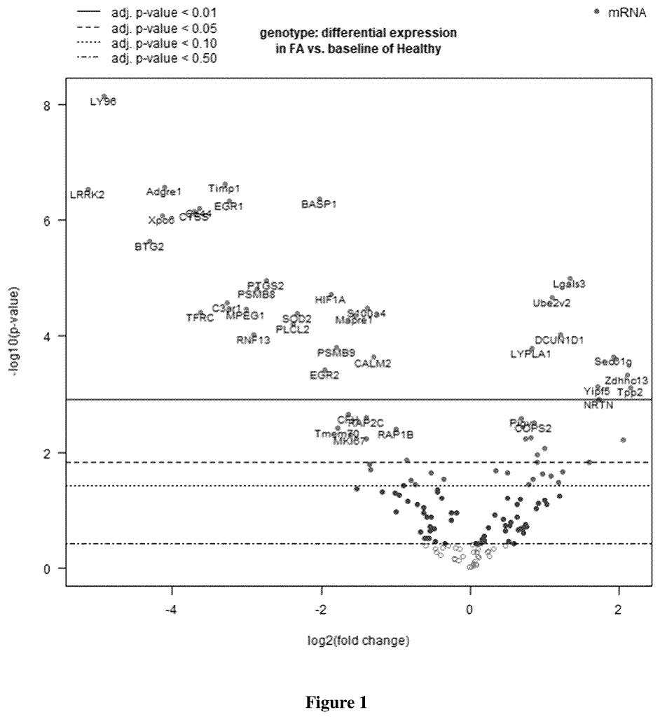

FIG. 1 is a volcano plot resulting from principal component analysis of buccal samples obtained from healthy subjects vs. buccal samples obtained from FRDA subjects who were not treated with FXN replacement therapy. FIG. 1 shows expression levels of various gene markers vs. their corresponding adjusted p-values. Based on the principal component analysis, FSGMs were identified as differentially expressed genes between the two groups.

FXN-sensitive genomic markers (FSGMs) were defined as differentially expressed genes between healthy subjects and subjects with FRDA that showed expression fold change of greater than 2 between groups, with Benjamini-Hochberg (BH) adjusted p-value <0.05. See the annotated genes above the dotted line corresponding to the adjusted p-value <0.05 in FIG. 1 that are also listed in Table 1 below. Some of the genes with fold change in expression levels of less than 2, e.g., between 1 and 2, or an adjusted p-value of larger than 0.05 were also identified as FSGMs after further validation (e.g., Lgals3 and PSMB9).

TABLE 1A
FXN-sensitive genomic markers (FSGMs)

| | Log2 fold change in expression | |
|---|---|---|
| Gene | level healthy vs. FRDA subjects | BH adjusted p-value |
| LY96 | 4.92 | 1.14E−06 |
| LRRK2 | 5.14 | 1.13E−05 |
| Adgre1 | 4.11 | 1.13E−05 |
| Timp1 | 3.29 | 1.13E−05 |
| Xpo6 | 4.14 | 1.45E−05 |
| CD44 | 3.64 | 1.38E−05 |
| BASP1 | 2.02 | 1.23E−05 |
| PSMB8 | 2.86 | 0.000188 |
| C3ar1 | 3.26 | 0.000265 |
| TFRC | 3.62 | 0.000319 |
| SOD2 | 2.33 | 0.000319 |
| PLCL2 | 2.38 | 0.000436 |
| S100a4 | 1.38 | 0.000301 |
| Mapre1 | 1.55 | 0.000327 |
| PSMB9 | 1.8 | 0.000984 |
| Lgals3 | −1.34 | 0.000142 |
| Ube2v2 | −1.1 | 0.000223 |
| Sec61g | −1.92 | 0.00132 |
| Zdhhc13 | −2.11 | 0.00246 |
| Yipf5 | −1.71 | 0.00373 |
| Tpp2 | −2.15 | 0.0038 |
| CFH | 1.64 | 0.0104 |
| Tmem70 | 1.78 | 0.0162 |
| Ybx2 | −2.06 | 0.0218 |
| Ptms | −1 | 0.0307 |

Additional genes that were demonstrated to be FSGMs are shown in Table 4 below.

TABLE 4
Additional FSGMs

| Gene | Log2 fold change in expression level | BH adjusted p-value |
|---|---|---|
| BTG2 | 4.3 | 3.63E−05 |
| CTSS | 3.71 | 1.38E−05 |
| EGR1 | 3.23 | 1.23E−05 |
| PTGS2 | 2.74 | 0.000142 |
| HIF1A | 1.87 | 0.000211 |
| MPEG1 | 3.01 | 0.000307 |
| RNF13 | 2.91 | 0.000623 |
| EGR2 | 1.95 | 0.00203 |
| CALM2 | 1.29 | 0.00132 |
| DCUN1D1 | −1.22 | 0.000623 |
| NRTN | −1.72 | 0.006 |
| RAP2C | 1.4 | 0.0113 |
| MKI67 | 1.61 | 0.0201 |
| EIF1AX | −1.6 | 0.0485 |
| Kctd12b | 1.39 | 0.0218 |

FIG. 2 is a box plot showing the relative levels of exemplary gene markers in healthy subjects (“Normal”) and FRDA subjects treated with placebo (“FA placebo”). FIG. 2 further demonstrates that there are differences in the relative levels of gene markers identified as FSGMs in buccal samples obtained from healthy subjects and FRDA subjects (see, e.g., LRRK2, SOD2, BTG2, CD44, EGR1, Lgals3, PSMB9, PTGS2 and Xpo6). FIG. 2 also shows exemplary gene markers that show no differences in the relative levels in buccal samples obtained from healthy subjects and FRDA subjects, and thus were not identified as FSGMs (e.g., ACTB, ATP6V0C, ATF4 and Rps28).

The results presented in FIGS. 1 and 2 and Tables 1 and 4 indicate that the expression levels of gene markers identified as FSGMs in this study are correlated with FXN levels in a subject.

### Example 2. Evaluation of FSGMs Following FXN Replacement Therapy

The FSGMs identified in Example 1 were further evaluated to identify FSGMs that are modulated in FRDA subjects upon treatment with FXN replacement therapy, such that their expression levels are modulated towards levels found in healthy subjects, e.g., become more similar or approach an equivalent level to those of healthy subjects after the FXN replacement as compared to before the therapy. To this end, the expression levels of the FSGMs in healthy subjects were compared to the expression levels of the FSGMs in FRDA subjects treated with placebo or with FXN replacement therapy, specifically the exemplary FXN fusion protein described in Example 1 (SEQ ID NO: 12). The data for FSGMs found to be modulated upon FXN replacement therapy are shown in Table 5 below. Specifically, Table 5 shows the results of two comparisons: a) a comparison of the gene expression levels in healthy subjects vs. FRDA subjects who were not treated with FXN replacement therapy (i.e., the same data shown in Table 1); and b) a comparison of gene expression levels in FRDA subjects pre-dose vs. FRDA subjects treated with 100 mg of the exemplary FXN fusion protein, in the buccal samples from the subjects taken on day 22 (i.e., after 13 days of dosing and 9 days after the last dose).

TABLE 5
FSGMs modulated in subjects dosed with an FXN fusion protein

| | | | FRDA subjects dosed with 100 | |
|---|---|---|---|---|
| | Healthy subjects vs. FRDA | | mg of the exemplary FXN fusion | |
| | subjects, pre-dose | | protein, day 22 vs pre-dose | |
| | Log2 fold | BH adjusted | Log2 fold | BH adjusted |
| Gene | change | p-value | change | p-value |
| Timp1 | 3.29 | 1.13E−05 | 3.1 | 0.00867 |
| LRRK2 | 5.14 | 1.13E−05 | 4.93 | 0.00411 |
| BASP1 | 2.02 | 1.23E−05 | 1.4 | 0.0117 |
| CD44 | 3.64 | 1.38E−05 | 3.28 | 0.00543 |
| Xpo6 | 4.14 | 1.45E−05 | 5.08 | 0.000247 |
| Lgals3 | −1.34 | 0.000142 | −1.02 | 0.00556 |
| SOD2 | 2.33 | 0.000319 | 2.52 | 0.00265 |
| PSMB9 | 1.8 | 0.000984 | 1.69 | 0.032 |

The results presented in Table 5 indicate that expression levels of each of Timp1, LRRKs, BASP1, CD44, Xpo6, SOD2 and PSMB9 are decreased in buccal samples obtained from FRDA subjects as compared to healthy subjects, and, conversely, expression levels of each are increased in FRDA subjects following FXN replacement therapy. The results presented in Table 5 further show that the expression level of Lgals3 is increased in buccal samples obtained from FRDA subjects as compared to healthy subjects, and, conversely, is decreased in FRDA subjects following FXN replacement therapy.

The two comparisons presented in Table 5 above were also made for the gene markers listed in Table 4. The results of the comparisons are presented in Table 6 below.

TABLE 6
Additional FSGMs modulated in subjects
dosed with an FXN fusion protein

| | | | FRDA subjects dosed with 100 | |
|---|---|---|---|---|
| | Healthy subjects vs. | | mg of the exemplary FXN fusion | |
| | FRDA subjects, pre-dose | | protein, day 22 vs. pre-dose | |
| | Log2 fold | BH adjusted | Log2 fold | BH adjusted |
| Gene | change | p-value | change | p-value |
| EGR1 | 3.23 | 1.23E−05 | 3.7 | 0.00151 |
| CTSS | 3.71 | 1.38E−05 | 3.99 | 0.0107 |
| BTG2 | 4.3 | 3.63E−05 | 5.15 | 0.00177 |
| PTGS2 | 2.74 | 0.000142 | 3.27 | 0.00109 |

The results presented in Table 6 show that the expression levels of each of EGR1, CTSS, BTG2 and PTGS2 are decreased in buccal samples obtained from FRDA subjects as compared to healthy subjects, and, conversely, are increased in FRDA subjects following FXN replacement therapy.

In addition, the gene markers PSMB8, MPEG1, TFRC, RNF13, PSMB9, EGR2, RAP2C and Kctd12b were also observed to be differentially expressed in healthy subjects as compared to FXN deficient subjects, and contrary regulated following FXN replacement therapy, as is evidenced by the data presented in Table 7.

TABLE 7
Additional gene markers modulated in subjects
dosed with an FXN fusion protein

| | | | FRDA subjects dosed with 100 | |
|---|---|---|---|---|
| | Healthy subjects vs. | | mg of the exemplary FXN fusion | |
| | FRDA subjects, pre-dose | | protein, day 22 vs. pre-dose | |
| | Log2 fold | BH adjusted | Log2 fold | BH adjusted |
| Gene | change | p-value | change | p-value |
| PSMB8 | 2.86 | 0.000188 | 3.51 | 0.00212 |
| TFRC | 3.62 | 0.000319 | 3.7 | 0.0117 |
| MPEG1 | 3.01 | 0.000307 | 3.89 | 0.0117 |
| RNF13 | 2.91 | 0.000623 | 3.12 | 0.0474 |
| EGR2 | 1.95 | 0.00203 | 2.14 | 0.0107 |
| RAP2C | 1.4 | 0.0113 | 1.17 | 0.032 |
| Kctd12b | 1.39 | 0.0218 | 2.82 | 0.032 |

The results presented in Table 7 show that the expression levels of each of PSMB8, TFRC, MPEG1, RNF13, EGR2, RAP2C and Kctd12b are decreased in buccal samples obtained from FRDA subjects as compared to healthy subjects, and, conversely, are increased in FRDA subjects following FXN replacement therapy.

The data presented in Tables 5 and 6 is also presented in FIG. 3. Specifically, FIG. 3 is a volcano plot resulting from principal component analysis of samples obtained pre-dose (day −2) and on day 22 from subjects treated with 100 mg of the exemplary FXN fusion protein. FIG. 3 shows expression levels of various gene markers vs. their corresponding adjusted p-values.

Taken together, the results presented in FIG. 3 and Tables 5, 6 and 7 demonstrate that the expression levels of certain FSGMs that were decreased in FRDA subjects (pre-dose) as compared to healthy subjects (e.g., LRRK2 and Xpo6, see also FIG. 1) are increased on day 22 in FRDA subjects who were treated with 100 mg of the exemplary FXN fusion protein. Similarly, the expression levels of certain FSGMs that were increased in FRDA subjects (pre-dose) as compared to healthy subjects (e.g., Lgals3, see also FIG. 1) are decreased on day 22 in FRDA subjects who were treated with 100 mg of the exemplary FXN fusion protein. Thus, the results presented in FIG. 3 and Tables 5, 6 and 7 demonstrate that administration of an FXN replacement therapy (e.g., an FXN fusion protein) to FRDA subjects causes several FSGMs to be modulated in such a way as to approach levels seen in healthy subjects.

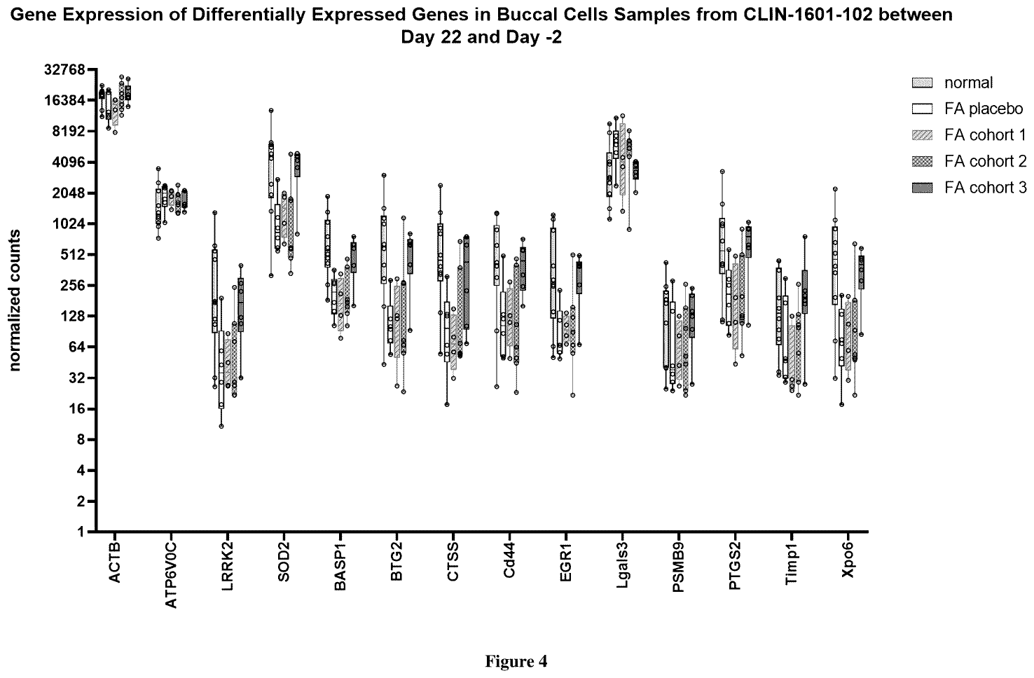

Next, the relative amounts of selected genes were compared in healthy subjects and FRDA subjects on day 22 after treatment with different, increasing doses of the FXN replacement therapy (cohorts 1, 2 or 3). The results of the comparisons are presented in FIG. 4. Specifically, FIG. 4 is a box plot showing relative amounts of gene markers in buccal samples obtained from healthy subjects (“Normal”) and FRDA subjects on day 22 after treatment with placebo (“FA placebo”), 25 mg (“FA cohort 1”), 50 mg (“FA cohort 2”) and 100 mg (“FA cohort 3”) of the exemplary FXN fusion protein. The results presented in FIG. 4 show that levels of FSGMs, such as LRRK2, SOD2, BASP1, BTG2, CTSS, CD44, EGR1, PSMB9, PTGS2, Timp1 and Xpo6, are decreased in FRDA subjects as compared to healthy subjects, and are increased in FRDA subjects after FXN replacement therapy (day 22) in a dose-dependent manner towards or equivalent to levels seen in healthy subjects. Similarly, the results presented in FIG. 4 demonstrate that level of Lgals3 is increased in FRDA subjects as compared to healthy subjects, and is decreased in FRDA subjects after FXN replacement therapy (day 22) in a dose-dependent manner towards or equivalent to levels seen in healthy subjects. The results presented in FIG. 4 also show that levels of exemplary genes, ACTB and ATP6V0C, which have not been identified as FSGMs, are not affected by FXN levels, or by treatment with FXN replacement therapy.

In summary, the results presented in this Example indicate that expression levels of certain gene markers identified as FSGMs are modulated in FRDA subjects upon treatment with FXN replacement therapy, such that their levels become similar to those of healthy subjects after the FXN replacement therapy.

A number of genes were identified as differentially expressed in FRDA subjects pre-dose as compared to healthy subjects, but were not observed to respond significantly to FXN replacement therapy when analyzed in buccal samples obtained from the FRDA subjects in this particular study, i.e., were not observed to be contrary regulated. It is noted that the ability to experimentally detect modulation of gene expression following FXN replacement therapy of certain genes may be dependent on, for example, the time of sampling relative to dosing, dosage and/or administration schedule of the FXN replacement therapy, the tissue being sampled (as genes may be differentially expressed in different tissues), and/or the sensitivity of the experimental technique used to measure the gene expression (e.g., Nanostring or qPCR). Thus, it is predicted that all of the differentially expressed gene markers could be modulated by FXN replacement therapy, e.g., be contrary regulated, and such modulation detected under appropriate conditions (e.g., a different dosing, administration schedule and/or time point after the FXN replacement therapy, in a different tissue sample, and/or by employing a technique other than Nanostring, e.g., qPCR).

### Example 3. Time-Dependent Changes in Expression Levels of FSGMs Following FXN Replacement Therapy

The goal of this experiment was to examine temporal changes in the expression levels of FSGMs in FRDA subjects following FXN replacement therapy. To this end, the levels of various gene markers identified as FSGMs were compared in buccal samples obtained on day 2, day 15, day 22 and day 43 following dosing with placebo or 25 mg, 50 mg or 100 mg of the exemplary FXN fusion protein (SEQ ID NO: 12).

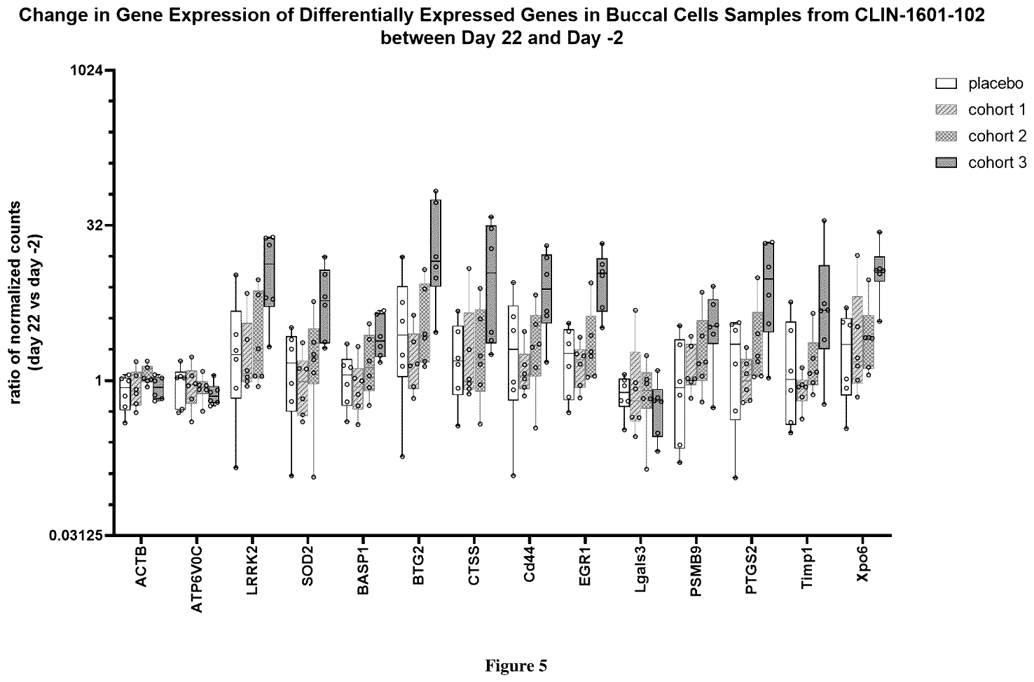

FIG. 5 presents the data for day 22. Specifically, FIG. 5 is a box plot showing the ratio of the relative amounts of gene markers in FRDA subjects treated with placebo (“placebo”), 25 mg (“cohort 1”), 50 mg (“cohort 2”) and 100 mg (“cohort 3”) of the exemplary FXN fusion protein on day 22 after dosing as compared to pre-dose. The results presented in FIG. 5 demonstrate that there is a dose-dependent change in the expression levels of validated FSGMs from day 1 to day 22. The results indicate that the levels of these FSGMs are responsive to the FXN replacement therapy in a dose- and time-dependent manner.

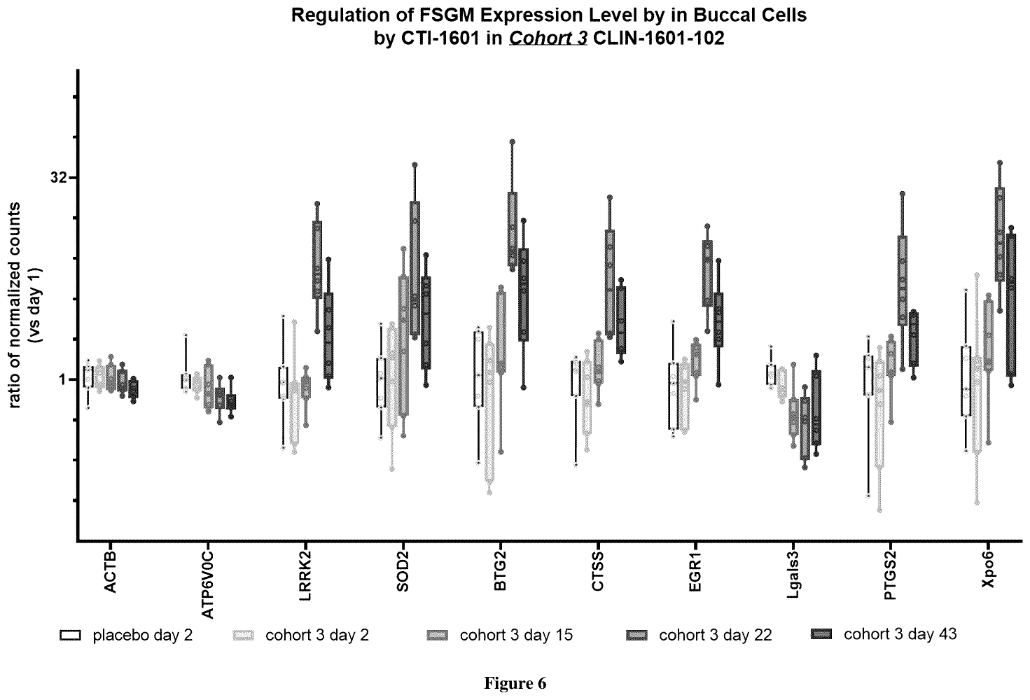

The results for samples obtained on day 2, day 15, day 22 and day 43 are presented in FIG. 6. Specifically, FIG. 6 is a box plot showing the relative expression levels of various FSGMs on day 2, day 15, day 22 and day 43 measured in buccal samples taken after dosing FRDA subjects with placebo or 100 mg of the exemplary FXN fusion protein (“cohort 3”). The results presented in FIG. 6 demonstrate that there is a time-dependent change in the levels of various FSGMs on different days after dosing. Specifically, the levels of FSGMS, such as LRRK2, SOD2, BTG2, CTSS, EGR2, PTGS2 and Xpo6, increase in a time-dependent and dose-dependent manner up to day 22 following FXN replacement therapy, and start to decrease after day 22. Similarly, the level of Lgals3 decreases in a time-dependent and dose-dependent manner up to day 22 following FXN replacement therapy, and starts to increase after day 22. The results presented in FIG. 6 indicate that the greatest time-dependent change in the levels of FSGMs is seen on day 22 in this study. Thus, temporal changes in the expression levels of various FSGMs may be used to determine and adjust the dosing and dosing regimen of FXN replacement therapy.

Levels of these selected gene markers identified as FSGMs were also measured over time in healthy subjects. It was found that the expression levels of the FSGMs in healthy subjects remain relatively stable, i.e., do not substantially change, over time, contributing to their utility as reliable biomarkers.

### Example 4. Measurement of FSGMs in Blood Samples

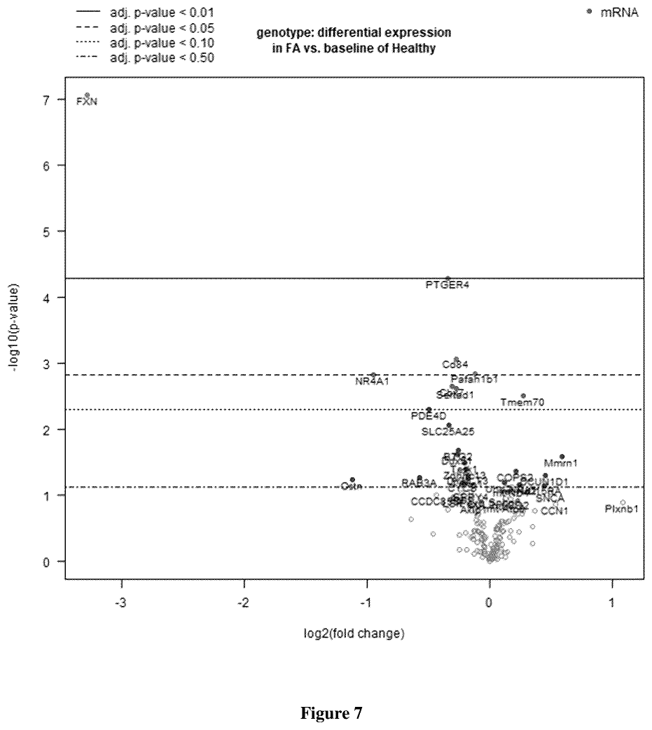

In Examples 1-3, FSGMs were measured in buccal samples. The goal of this experiment was to determine if modulation of the FSGMs identified herein may also be detected in blood samples. To this end, levels of FSGMs were measured in blood samples obtained from healthy subjects and FRDA subjects who were not treated with FXN replacement therapy. The results of the experiment are presented in FIG. 7. Specifically, FIG. 7 is a volcano plot resulting from principal component analysis of whole blood samples obtained from healthy subjects vs. FRDA subjects who were not treated with FXN replacement therapy. FIG. 7 shows expression levels of various gene markers vs. their corresponding adjusted p-values. The results presented in FIG. 7 indicate that principal component analysis of gene expression data obtained by analysis of blood samples does not discriminate between healthy subjects and FRDA subjects.

### Example 5. Evaluation of Additional FSGMs Following FXN Replacement Therapy

The goal of this experiment was to identify additional FSGMs using the samples from Example 1 by re-analyzing the samples using a different codeset than that used in Example 1. This additional analysis yielded identification of FTL and TXNIP as additional FSGMs.

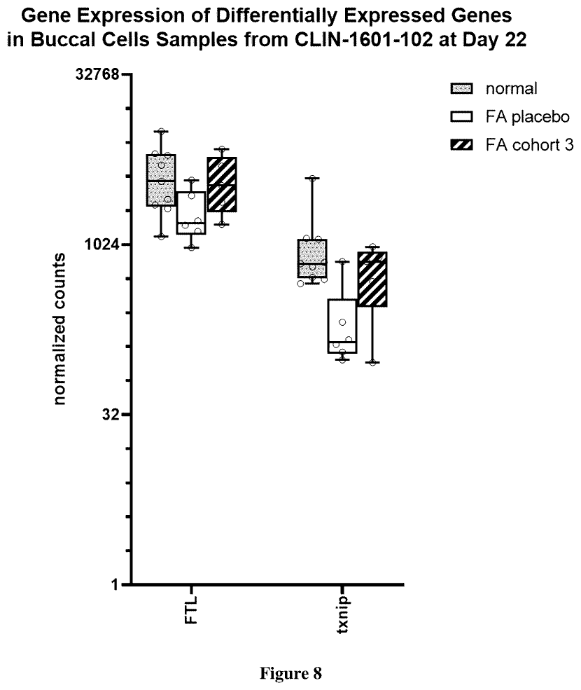

The results of the additional analysis are presented in FIG. 8. Specifically, FIG. 8 is a box plot showing relative amounts of FTL and TXNLP gene markers in buccal samples obtained from healthy subjects (“Normal”) and FRDA subjects on day 22 after treatment with placebo (“FA placebo”) and 100 mg (“FA cohort 3”) of the exemplary FXN fusion protein. The results presented in FIG. 8 show that levels of FTL and TXNLP are decreased in FRDA subjects as compared to healthy subjects, and are increased in FRDA subjects after FXN replacement therapy (day 22) towards or equivalent to levels seen in healthy subjects.

Temporal changes in the expression levels of FTL and TXNLP in FRDA subjects following FXN replacement therapy were also examined Specifically, the levels of FTL and TXNLP were compared in buccal samples obtained pre-dose and on day 22 following dosing with placebo or 100 mg of the exemplary FXN fusion protein.

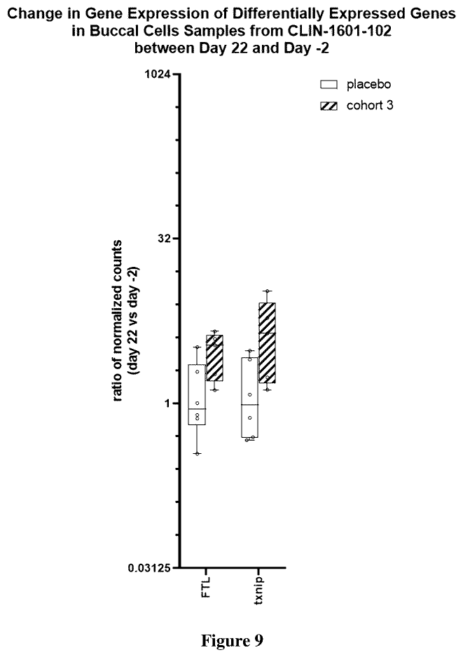

FIG. 9 is a box plot showing the ratio of the relative amounts of gene markers in FRDA subjects treated with placebo (“placebo”) and 100 mg (“cohort 3”) of the exemplary FXN fusion protein on day 22 after dosing as compared to pre-dose. The results presented in FIG. 9 demonstrate that there is a change in the expression levels of FTL and TXNLP from pre-dose to day 22. The results indicate that the levels of these FSGMs are responsive to the FXN replacement therapy in a time-dependent manner.

In summary, the results presented in this Example indicate that expression levels of FTL and TXNLP are modulated in FRDA subjects upon treatment with FXN replacement therapy, such that their levels become similar to those of healthy subjects after the FXN replacement therapy.

Descriptions of embodiments of the disclosure in the present application are provided by way of example and are not intended to limit the scope of the disclosure. The described embodiments comprise different features, not all of which are required in all embodiments. Some embodiments utilize only some of the features or possible combinations of the features. Variations of embodiments of the disclosure that are described, and embodiments comprising different combinations of features noted in the described embodiments, will occur to persons of the art. The scope of an embodiment of the disclosure is limited only by the claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

1. A method for evaluating efficacy of a frataxin (FXN) replacement therapy, the method comprising:
(a) determining a baseline FXN(−) expression profile for one or more FXN-sensitive genomic markers (FSGMs) in a sample obtained from an FXN deficient subject prior to administration of the FXN replacement therapy;
(b) determining an FXN replacement expression profile for the one or more FXN-sensitive genomic markers (FSGMs) in a sample obtained from the FXN deficient subject following administration of the FXN replacement therapy;
(c) comparing the FXN replacement expression profile determined in step (b) with the baseline FXN(−) expression profile determined in step (a); and
(d) determining efficacy of the FXN replacement therapy based on the comparison in step (c);
wherein the one or more FSGMs are selected from the markers defined in Table 1.

2-41. (canceled)

42. A method of monitoring treatment of a subject with a frataxin (FXN) replacement therapy, the method comprising:
(a) determining a first FXN replacement expression profile for one or more FXN-sensitive genomic markers (FSGMs) in a first sample obtained from an FXN deficient subject at a first time point following administration of an FXN replacement therapy to the subject, wherein the one or more FSGMs comprises one or more markers defined in Table 1;
(b) determining a second FXN replacement expression profile for the one or more FXN-sensitive genomic markers (FSGMs) in a second sample obtained from the subject at a second time point that is later than the first time point;
(c) comparing the second FXN replacement expression profile with the first FXN replacement profile; thereby monitoring treatment of the subject with the FXN replacement therapy.

43-45. (canceled)

46. A method for treating an FXN deficiency in a subject, the method comprising:
(a) determining an FXN expression profile in a sample obtained from an FXN deficient subject for one or more FXN-sensitive genomic markers (FSGMs);
(b) comparing the FXN expression profile of the sample with at least one other expression profile selected from the group consisting of normal FXN expression profile for the one or more FSGMs, baseline FXN(−) expression profile for the one or more FSGMs, and FXN replacement expression profile for the one or more FSGMs;
(c) classifying the FXN expression profile determined in step (a) as corresponding to a normal FXN expression profile, baseline FXN(−) expression profile or an FXN replacement expression profile; and
(d) initiating or modulating an FXN replacement therapy based on the classification of the FXN expression profile of the sample;
wherein the one or more FSGMs comprises one or more markers defined in Table 1.

47. The method of claim 46, wherein modulating an FXN replacement therapy comprises increasing the dosage, decreasing the dosage, increasing the administration frequency, or decreasing the administration frequence of the FXN replacement therapy.

48. The method of claim 46, wherein the FXN deficient subject has Friedrich's Ataxia (FRDA).

49. (canceled)

50. A method of treating an FXN deficiency in a subject, comprising:
(a) obtaining an FXN expression profile for one or more FSGMs in a sample obtained from an FXN deficient subject; and
(b) administering an FXN replacement therapy to the subject based on the subject FXN expression profile.

51. The method of claim 50, further comprising obtaining the sample from the FXN deficient subject for use in determining the FXN expression profile for the one or more FSGMs.

52. A method of detecting one or more frataxin-sensitive genomic markers (FSGMs) in a sample from a frataxin (FXN) deficient subject, comprising contacting the sample, or a portion thereof, with one or more reagents specific for detecting the level of each of one or more FSGMs, wherein the one or more FSGMs comprise one or more FSGMs selected from Table 1, thereby detecting the FSGMs in the sample.

53. The method of claim 52, wherein the FXN deficient subject is undergoing treatment or is scheduled to be treated with an FXN replacement therapy.

54. The method of claim 52, further comprising obtaining the sample from the FXN deficient subject.

55. The method of claim 52, wherein the one or more FSGMs comprise one or more of Timp1, LRRK2, BASP1, CD44, Xpo6, Lgals3, SOD2, PSMB9, FTL and TXNIP.

56. (canceled)

57. The method of claim 52, wherein the one or more FSGMs further comprise one of more FSGMs defined in Table 2 or Table 3.

58. The method of claim 57, wherein the one or more FSGMs defined in Table 2 or Table 3 comprise one or more of CTSS, BTG2, EGR1 and PTGS2.

59. The method of claim 58, wherein the one or more FSGMs comprise Xpo6, SOD2, CTSS, EGR1, BTG2, PTSG2 and LRKK2.

60. The method of claim 59, wherein the one or more FSGMs comprise LRRK2, Xpo6, EGR1, BTG2, PTSG2 and LRKK2.

61. The method of claim 52, wherein the one or more FSGMs are detected by one or more of sequencing, hybridization and amplification of RNA in the sample.

62. The method of claim 52, wherein the one or more FSGMs are detected by HPLC/UV-Vis spectroscopy, enzymatic analysis, mass spectrometry, NMR, immunoassay, ELISA, or any combination thereof.

63. The method of claim 52, wherein the subject has Freidrich's Ataxia (FRDA).

64. (canceled)

65. The method of claim 54, wherein the sample is selected from the group consisting of a buccal sample, a skin sample, a hair follicle and a muscle biopsy sample.

66. The method of claim 54, wherein the sample is a buccal sample or a skin sample.

67. (canceled)

68. The method of claim 54, wherein said sample from the FXN deficient subject is obtained at least 15 days following the last administration of the FXN replacement therapy.

69. The method of claim 68, wherein said sample from the FXN deficient subject is obtained 15 to 45 days following the last administration of the FXN replacement therapy.

70. The method of claim 1, wherein the FXN replacement therapy comprises administration of an FXN fusion protein.

71. The method of claim 70, wherein the FXN fusion protein comprises or consists of the amino acid sequence set forth in SEQ ID NO: 12.

72. A kit for detecting one or more frataxin-sensitive genomic markers (FSGMs) in a sample obtained from a frataxin (FXN) deficient subject, comprising at least one reagent specific for detecting the level of each of the one or more FSGMs in the sample, wherein the one or more FSGMs comprises one or more FSGMs defined in Table 1, and a set of instructions for detecting the level of the one or more FSGMs in the sample from the subject.

73-78. (canceled)

79. A panel of reagents for use in a method of monitoring or evaluating the efficacy of frataxin (FXN) replacement therapy, the panel comprising at least two detection reagents, wherein each detection reagent is specific for the detection of at least one frataxin-sensitive genomic marker (FSGM) of a set of FSGMs, wherein the set of FSGMs comprises two or more markers defined in Table 1.

80-84. (canceled)

85. A kit comprising the panel of claim 79 and a set of instructions for obtaining information relating to frataxin (FXN) replacement therapy based on a level of the one or more frataxin-sensitive genomic markers (FSGMs).


SEQUENCE LISTING (12 sequences)

SEQ ID NO: 1 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 130 aa, Homo sapiens
SGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - protein, 80 aa, Homo sapiens
MWTLGRRAVAGLLASPSPAQAQTLTRVPRPAELAPLCGRRGLRTDIDATCTPRRASSNQRGLNQIWNVKKQSVYLMNLRK

SEQ ID NO: 4 - protein, 11 aa, Human immunodeficiency virus 1
YGRKKRRQRRR

SEQ ID NO: 5 - protein, 12 aa, synthetic construct
MYGRKKRRQRRR

SEQ ID NO: 6 - protein, 30 aa, synthetic construct
GWTLNSAGYLLGPHAVGNHRSFSDKNGLTS

SEQ ID NO: 7 - protein, 14 aa, synthetic construct
  SITE 14: note = Leucine amide
INLKALAALAKKIL

SEQ ID NO: 8 - protein, 27 aa, synthetic construct
GWTLNSAGYLLGKINLKALAALAKKIL

SEQ ID NO: 9 - protein, 16 aa, synthetic construct
RQIKIWFQNRRMKWKK

SEQ ID NO: 10 - protein, 9 aa, synthetic construct
RRRRRRRRR

SEQ ID NO: 11 - protein, 34 aa, synthetic construct
DAATATRGRSAASRPTERPRAPARSASRPRRPVE

SEQ ID NO: 12 - protein, 224 aa, synthetic construct
MYGRKKRRQRRRGGMWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA